(19) United States
(12) Patent Application Publication
(10) Pub. No.: US 2026/0242753 A1
(43) Pub. Date: Aug. 20, 2026

(54) NOVEL INHIBITORS OF HISTONE METHYLTRANSFERASE NUCLEAR LOCALISATION

(71) Applicant: The Council of the Queensland Institute of Medical Research, Herston, Brisbane, Queensland (AU)

(72) Inventor: Sudha Rao, Herston, Brisbane, Queensland (AU)

(21) Appl. No.: 18/992,665

(22) PCT Filed: Jul. 13, 2023
(86) PCT No.: PCT/AU2023/050647

§ 371 Date: Jan. 9, 2025

(30) Foreign Application Priority Data
Jul. 13, 2022 (AU) 2022901957

(51) Int. Cl. C12N 9/10; A61K 38/00; A61K 45/06; A61P 35/00; C07K 14/47; G01N 33/575

(52) U.S. Cl. CPC C12N 9/1007; A61K 45/06; A61P 35/00; C07K 14/47; G01N 33/5758; A61K 38/00; G01N 2333/91011

(57) ABSTRACT

The present invention relates to novel therapeutic compositions and methods for treating cancer. In particular, the use of proteinaceous inhibitors, including novel bicyclic peptide inhibitors, for use in treating cancer.

### FIELD OF THE INVENTION

This invention relates generally to compositions for at least partially inhibiting nuclear localisation of SETDB1. The invention also provides proteinaceous molecules corresponding to a nuclear localization site of SETDB1, and their use in preventing the nuclear localization of a SETDB1 polypeptide. This invention also relates to the use of the compositions and proteinaceous molecules for treating or preventing a cancer in a subject. This invention also relates generally to methods and agents for predicting response to therapy. More particularly, the present disclosure relates to methods, agents and kits for analysing cellular distribution of SETDB1, for stratifying a subject as a likely responder or non-responder to a therapy, particularly an immunotherapy.

Various bibliographic references referred to by number in the specification are listed at the end of the description.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

There now exists a body of evidence to illustrate how tumors co-opt certain immune-checkpoint pathways as a major mechanism of immune resistance, particularly against T cells that are specific for tumour antigens. Because many of the immune checkpoints are initiated by ligand-receptor interactions, they can be readily blocked by antibodies or modulated by recombinant forms of ligands or receptors. Thus agonists of co-stimulatory receptors or antagonists of inhibitory signals, both of which result in the amplification of antigen-specific T cell responses are the primary agent in current clinical testing.

In this context, cancer immunotherapy has been viewed as breakthrough in the field of cancer treatment, switching from targeting the tumour to targeting the immune system (Couzin-Frankel., Science. 2013. 342(6165):1432-3). The blockade of immune checkpoints with antibodies targeting PD1, PD-L1, or CTLA-4, has given promising clinical results and manageable safety profiles. However, only a small proportion of patients respond to these therapies. Thus, there is a need to improve cancer immunotherapies by new approaches and/or by combining anti-checkpoint antibodies with other treatments (see, Jenkins et al., BJC. 2018; 118, 9-16; Sharma et al., Cell. 2017; 168(4):707-723). Moreover, anti-checkpoint antibodies can induce side effects, mainly autoimmunity, such that implementing combination therapies which may help lower the administered doses, and consequently the adverse events, remains of invaluable medical help.

SET Domain Bifurcated Histone Lysine Methyltransferase 1 (SETDB1, also termed ESET, and KMT1A) is a H3K9 methyltransferase involved in gene silencing. In recent years, SETDB1 has been reported to be an oncogene in numerous cancers. Histone methyltransferases (HMT) are histone-modifying enzymes (e.g., histone-lysine N-methyltransferases and histone-arginine N-methyltransferases), that catalyze the transfer of one, two, or three methyl groups to lysine and arginine residues of histone proteins. The attachment of methyl groups occurs predominantly at specific lysine or arginine residues on histones H3 and H4. The class of lysine-specific histone methyltransferases is further subdivided into SET domain-containing and non-SET domain-containing. Methylation of the N-terminal lysine residues of histone H3, notably in position 4, 9, 27, 36 and 79 to form mono-, di-, or tri-methylated lysines, is highly documented. More than 30 histone methyltransferases have currently been described.

The use of inhibitors of DNMT or HDAC has also been recently proposed in combination with other cancer therapies such as immunotherapy (Wrangle et al., Oncotarget. 4(11): 2067-2079, Chiapinelli et al., Cell. 2015; 162(5):974-86; Licht, Cell. 2015; 162(5):938-9; and Sharma, supra). Indeed, it has been suggested that DNA demethylating agents may prime solid tumors to T-cell-mediated immune response and, therefore, may work in synergy with antitumor immunotherapy, such as checkpoint inhibitors (Roulois et al., Oncoimmunology. 2016; 5(3): e1090077). Also, incidental clinical findings suggest that non-small-cell carcinoma lung cancer patients pre-treated with 5-Azacytidine have better clinical response to subsequent anti-PD1 therapy (Juergens et al., Cancer Discov. 2011; 1: 598-607) and mice models of melanoma have been shown to respond better to the combination of 5-Azacytidine and anti-CTLA-4 antibody therapy than either 5-Azacytidine alone or anti-CTLA4 antibody therapy alone (see, Chiappinelli et al., Cell. 2015; 162: 974-86; and Roulois et al., Cell. 2015; 162: 961-973).

However, the role of such epigenetic modulators in cancer immunology and immunotherapy remains poorly understood. The of demethylating agents are diverse, and identification of genes, whose reactivation predicts or mediates response, remains elusive. Typically, immune modulatory effects of treatment with 5-Azacytidine, a DNMT, are complex and dependent on the clinical setting and type of patients (see, Frosig and Hadrup, Mediators Inflamm. 2015; 871641).

Accordingly, elucidation of the precise molecular mechanisms by which epigenetic modulators assert their effect on the immune response to a cancer or tumour are highly desired.

### SUMMARY OF THE INVENTION

The present disclosure is based in part on the finding that translocation of SETDB1 into the nucleus of cells, including tumour cells, correlates with increased severity of cancer, as well as resistance to therapy. Accordingly, the present inventors found that inhibitors of SETDB1 nuclear localization.

Accordingly, in one aspect of the invention, there is provided a method of inhibiting or reducing the nuclear localization of a SETDB1 polypeptide in a cell, the method comprising contacting the cell with an agent that inhibits the binding of the SETDB1 polypeptide with an importin-α polypeptide.

In yet another aspect, there is provided a method of inhibiting or reducing the nuclear localization of a SETDB polypeptide in a cell, the method comprising contacting the cell with a proteinaceous molecule comprising, consisting, or consisting essentially of an amino acid sequence corresponding to a nuclear localization sequence (NLS) of a SETDB1 polypeptide. In this regard, the present invention also includes mimetics comprising an amino acid sequence according to Formula I are particularly efficacious in inhibiting or reducing the nuclear localization of SETDB1. Accordingly, the inventors have conceived that SETDB1 peptide mimetics comprising an amino acid sequence according to Formula I can be used to inhibit nuclear localization of SETDB1

In a further aspect the invention provides a method of treating or preventing cancer in a subject, the method comprising administering to the subject an agent that inhibits or prevents the binding of a SETDB1 polypeptide with an importin-α polypeptide.

In some embodiments, the cancer is associated with at least some SETDB1 polypeptide being present in the cell nucleus. By way of an illustrative example, the cancer may be selected from the group comprising breast cancer, prostate cancer, lung cancer, bladder cancer, pancreatic cancer, colon cancer, liver cancer, metastatic brain cancers, melanoma, retinoblastoma; ovarian cancer, and renal cell carcinoma.

In yet another aspect, there is provided a method of producing an agent that inhibits or reduces nuclear localization of a SETDB1 polypeptide, the method comprising:
a) contacting a cell with an agent; and
b) detecting a reduction in or inhibition of the nuclear localization of the SETDB1 polypeptide in the cell relative to a normal or reference level of nuclear localization in the absence of the agent.

In some embodiments, the agent inhibits the binding of the SETDB1 polypeptide to the IMPα polypeptide, but does not inhibit the binding of any other polypeptide to the IMPα polypeptide.

In some embodiments, the agent directly binds to the to IMPα polypeptide.

In some embodiments, the agent reduces the amount of SETDB1 that is transported into the cell nucleus. Accordingly, the agent reduces the amount of SETDB1 that is present in the cell nucleus.

In some embodiments, the agent is a proteinaceous molecule comprising, consisting, or consisting essentially of an amino acid sequence corresponding to a NLS from a SETDB1 polypeptide.

In some embodiments, the proteinaceous molecule comprises, consists, or consists essentially of the amino acid sequence of Formula (I). Preferably, the proteinaceous molecule comprises, consists, or consists essentially of an amino acid sequence set forth in any one of SEQ ID NOs: 1-6, or has at least 85% sequence identity to a sequence set forth in any one of SEQ ID NOs: 1-6. Even more preferably, the proteinaceous molecule comprises, consists, or consists essentially of the amino acid sequence set forth in SEQ ID NO: 2 or SEQ ID NO: 6.

In some embodiments, the cell is a cancer stem cell or a non-cancer stem cell tumour cell.

In some embodiments, the cancer is selected from the group comprising breast cancer, prostate cancer, lung cancer, bladder cancer, pancreatic cancer, colon cancer, liver cancer, metastatic brain cancers, melanoma, retinoblastoma; ovarian cancer, and renal cell carcinoma.

In some embodiments, the proteinaceous molecule comprises, consists or consists essentially of an amino acid sequence corresponding to residues 206 to 232 of a human SETDB1 polypeptide (i.e., the human SETDB1 sequence of UniProtKB Accession No. Q15047). In some alternative embodiments, the proteinaceous molecule is a fragment of a nuclear localizable polypeptide.

In some preferred embodiments, the proteinaceous molecule comprises 50 amino acid residues or less.

In some embodiments, the proteinaceous molecule is distinguished from SETDB1 by the addition, deletion and/or substitution of at least one (e.g., 1, 2, 3, 4, 5, 6, 7, 8, 9 etc.) amino acid in residues 206 to 232 of SETDB1.

In another aspect, the present invention provides a method of producing a proteinaceous molecule that inhibits or reduces nuclear localization of a SETDB1 polypeptide, the method comprising:
a) contacting a cell with a proteinaceous molecule comprising, consisting or consisting essentially of an amino acid sequence corresponding to residues 206 to 232 of SETDB1; and
b) detecting a reduction in or inhibition of the nuclear localization of the SETDB1 polypeptide in the cell relative to a normal or reference level of nuclear localization in the absence of the proteinaceous molecule.

In still another aspect, the invention provides an isolated or purified proteinaceous molecule represented by Formula (I):

Z₁GKKRX₁KX₂WHX₃GTLIX₄IQTVGXSGKKX₆KVKZ₂ Formula (I)
Z₁ and Z₂ are independently absent or are independently selected from at least one of a proteinaceous moiety comprising from about 1 to about 50 amino acid residues (and all integer residues in between), and a protecting moiety;
X₁ is selected from Thr, Arg, and modified forms thereof;
X₂ is selected from Thr, Leu, and modified forms thereof;
X₃ is selected from Lys, Gly, and modified forms thereof;
X₄ is selected from Ala, Pro, and modified forms thereof;
X₅ is selected from Pro, Lys, and modified forms thereof; and
X₅ is selected from Tyr, Lys, and modified forms thereof.

In some embodiments, Z₁ is absent.

In some of the same embodiments and some alternative embodiments, Z₂ is absent.

In some of the same embodiments and some alternative embodiments, X₁ is Thr.

In some of the same embodiments and some alternative embodiments, X₂ is Thr.

In some of the same embodiments and some alternative embodiments, X₃ is Lys.

In some of the same embodiments and some alternative embodiments, X₄ is Ala.

In some of the same embodiments and some alternative embodiments, X₅ is Pro.

In some of the same embodiments and some alternative embodiments, X₆ is Tyr.

In some of the same embodiments and some alternative embodiments, X₁ is Arg.

In some of the same embodiments and some alternative embodiments, X₂ is Leu.

In some of the same embodiments and some alternative embodiments, wherein X₃ is Gly.

In some of the same embodiments and some alternative embodiments, X₄ is Pro.

In some preferred embodiments, the proteinaceous molecule comprises, consists, or consists essentially of, an amino acid sequence set forth in SEQ ID NO: 1 or SEQ ID NO: 2.

In some preferred embodiments, the proteinaceous molecule of Formula I further comprises at least one membrane permeating moiety. By way of an example, the membrane permeating moiety may be a lipid moiety. In some embodiments of this type, the membrane permeating moiety is a myristoyl group.

In some embodiments, the membrane permeating moiety may be coupled to the N- or C-terminal amino acid residue. Preferably, the membrane permeating moiety is coupled to the N-terminal amino acid residue.

Additionally, the present inventors found that nuclear localized SETDB1 (also referred to herein as “nuclear SETDB1” or “intranuclear SETDB1”) co-localizes with at least one nuclear polypeptide (e.g., ATF7IP) and that this co-localization is a surrogate marker for increased disease severity and resistance to therapy (e.g., immunotherapy). These findings have been reduced to practice in methods and kits for predicting the likelihood of response to therapy in a subject, as described hereafter.

Accordingly, in one aspect, the present disclosure provides methods for predicting the likelihood of response to a therapy (e.g., immunotherapy) in a subject. These methods generally understood, consist, or consist essentially of analysing cellular localization of SETDB1 in a SETDB1-expressing cell of the subject, to thereby predict the likelihood of response of the subject to the therapy. The SETDB1-expressing cell is suitably a tumour cell. The therapy may be immunotherapy.

In some embodiments, the methods include detecting presence of SETDB1 in the nucleus of the cell or a level of SETDB1 in the nucleus of the cell, which is indicative of an aberrant or abnormal nuclear level of SETDB1 and/or which correlates with an increased likelihood of resistance to the therapy, to thereby determine that the subject has increased likelihood of resistance to the therapy. In some embodiments, the methods comprise detecting a higher level of SETDB1 relative to a control in the nucleus of the cell, to thereby determine that the subject has increased likelihood of resistance to the therapy. In some embodiments, the methods comprise comparing the level of SETDB1 between different cellular components (e.g., nucleus and cytoplasm), to thereby determine that the subject has increased likelihood of resistance to the therapy.

Suitably, the methods comprise detecting a higher level of SETDB1 in the nucleus of the cell relative to a control (e.g., relative to the nucleus of a corresponding normal control cell, or relative to the level of SETDB1 in the cytoplasm of the cell), which indicates that the subject has increased likelihood of resistance to the therapy. In non-limiting examples of these embodiments, the higher level of SETDB1 in the nucleus of the cell represents a level that is at least about 120%, 130%, 140% 150%, 160%, 170%, 180%, 190%, 200%, 250%, 300%, 400%, 500%, 600%, 700%, 800%, 900%, or 1000% (and every integer in between) of the level of SETDB1 in the nucleus of the corresponding normal control cell. In some of the same or other non-limiting examples of these embodiments, the higher level of SETDB1 in the nucleus of the cell represents a higher level of SETDB1 in the nucleus of the cell than outside the nucleus (e.g., in the cytoplasm, referred to herein as “cytoplasmic”) of the cell. In representative examples of this type, the higher level is indicative of a ratio of nuclear SETDB1 to cytoplasmic SETDB1 of greater than about 0.55, 0.60, 0.65, 0.70, 0.75, 0.85, 0.90 or 0.95. In some of the same and other non-limiting examples, the methods include detecting a higher level of nuclear SETDB1 in more than 30%, 35%, 40%, 45%, 50%, 55%, 60%, 65%, 70%, 75%, 80%, 85%, 90%, or 95% of the subject' cells (e.g., tumour cells).

In other embodiments, the methods include detecting an absence of SETDB1 in the nucleus of the cell or a level of SETDB1 in the nucleus of the cell, which is indicative of a normal nuclear level of SETDB1 and which correlates with an increased likelihood of sensitivity to the therapy, to thereby determine that the subject has increased likelihood of sensitivity to the therapy. In some of the same and other embodiments, the methods comprise detecting a level of SETDB1 in the nucleus of the cell relative to a control (e.g., relative to the nucleus of a corresponding normal cell, or relative to the levels of cytoplasmic SETDB1 in the cell), which level is indicative of a normal nuclear level of SETDB1 and which indicates that the subject has increased likelihood of sensitivity to the therapy. In some of the same and other embodiments, the methods comprise detecting presence of cytoplasmic SETDB1 in the cell to thereby determine that the subject has increased likelihood of sensitivity to the therapy. Suitably, the methods comprehend detecting a level of cytoplasmic SETDB1 in the cell relative to a control (e.g., relative to cytoplasmic SETBD1 of a corresponding normal cell, or relative to the levels of SETDB1 inside the nucleus of the subject's cell), which level is indicative of a normal extranuclear level of SETDB1 and which indicates that the subject has increased likelihood of sensitivity to the therapy. In non-limiting examples of these embodiments, the level of cytoplasmic SETDB1 of the cell represents a level that is about the same level (e.g., a level that is from about 85% to about 115%, and every integer in between) of cytoplasmic SETDB1 of the corresponding normal control cell. In some of the same or other non-limiting examples of these embodiments, the level of cytoplasmic SETDB1 of the cell represents a higher level of cytoplasmic SETDB1 than nuclear. In representative examples of this type, the higher level is indicative of a ratio of cytoplasmic SETDB1 to nuclear SETDB1 of greater than about 0.55, 0.60, 0.65, 0.70, 0.75, 0.85, 0.90 or 0.95. In some of the same and other non-limiting examples, the methods include detecting a normal level of cytoplasmic SETDB1 in more than 30%, 35%, 40%, 45%, 50%, 55%, 60%, 65%, 70%, 75%, 80%, 85%, 90%, or 95% of the subject's cells.

Suitably, in any of the above embodiments, the methods comprise detecting co-localization of SETDB1 with a nuclear binding partner of SETDB1 (e.g., ATF7IP). In some examples of these embodiments, the methods comprehend contacting a sample comprising a cell of the subject or lysate of the cell with a first antigen-binding molecule that binds specifically to SETDB1 and a second antigen-binding molecule that binds specifically to the nuclear binding partner (e.g., ATF7IP), and detecting the presence in the sample of a complex that comprises the first antigen-binding molecule and the second antigen-binding molecule, to thereby determine that the subject has increased likelihood of resistance to the therapy. In some embodiment, the methods comprise detecting a higher level of the complex relative to a control (e.g., a corresponding normal control cell), which indicates that the subject has increased likelihood of resistance to the therapy. In other embodiments, the methods comprise detecting a level of the complex in the nucleus relative to a control (e.g., a corresponding normal or immunocompetent control cell), which level is indicative of a normal level of the complex and which indicates that the subject has increased likelihood of sensitivity to the therapy.

Another aspect of the present disclosure provides methods for determining likelihood of resistance to a therapy (e.g., immunotherapy) in a subject. These methods generally understood, consist or consist essentially of detecting in a sample (e.g., a sample comprising a SETDB1-expressing cell such as or tumour cell, or lysate thereof) of the subject co-localization of SETDB1 with a nuclear binding partner of SETDB1 (e.g., ATF7IP), or a level of the co-localization, which is indicative of an aberrant or abnormal level of the co-localization and which correlates with an increased likelihood of resistance to the therapy, to thereby determine that the subject has increased likelihood of resistance to the therapy. In some embodiments, the methods comprise detecting in the sample a higher level of the co-localization relative to a control (e.g., a reference sample comprising a corresponding normal control SETDB1-expressing cell, or lysate thereof), to thereby determine that the subject has increased likelihood of resistance to the therapy. In other embodiments, the methods comprise detecting in the sample about the same level of the co-localization relative to a control (e.g., a reference sample comprising a corresponding SETDB1-expressing cell having an aberrant level of nuclear SETDB1, or lysate thereof), to thereby determine that the subject has increased likelihood of resistance to the therapy.

In a related aspect, the present disclosure provides methods for determining likelihood of resistance to a therapy (e.g., immunotherapy) in a subject. These methods generally understood, consist or consist essentially of detecting in a sample (e.g., a sample comprising a SETDB1-expressing cell such as a tumour cell, or lysate thereof) of the subject presence of a complex comprising SETDB1 and a nuclear binding partner of SETDB1 (e.g., ATF7IP), or a level of the complex, which is indicative of an aberrant or abnormal level of the complex and which correlates with an increased likelihood of resistance to the therapy, to thereby determine that the subject has increased likelihood of resistance to the therapy.

In some embodiments, the methods comprise detecting in the sample a higher level of the complex relative to a control (e.g., a reference sample comprising a corresponding normal control SETDB1-expressing cell, or lysate thereof), to thereby determine that the subject has increased likelihood of resistance to therapy.

In other embodiments, the methods comprise detecting about the same level of the complex relative to a control (e.g., a reference sample comprising a corresponding SETDB1-expressing cell having an aberrant level of nuclear SETDB1, or lysate thereof), to thereby determine that the subject has increased likelihood of resistance to the therapy.

Yet another aspect of the present disclosure provides methods for determining the likelihood of sensitivity to a therapy (e.g., immunotherapy) in a subject. These methods generally consist or consist essentially of detecting in a sample (e.g., a sample comprising a SETDB1-expressing cell such as a tumour cell, or lysate thereof) of the subject absence of co-localization of SETDB1 with a nuclear binding partner of SETDB1 (e.g., ATF7IP), or a level of the co-localization, which is indicative of a normal level of the co-localization and which correlates with an increased likelihood of sensitivity to the therapy, to thereby determine that the subject has increased likelihood of sensitivity to the therapy. In some embodiments, the methods comprise detecting about the same level of the co-localization relative to a control (e.g., a reference sample comprising a corresponding normal control SETDB1-expressing cell, or lysate thereof) in a sample (e.g., a sample comprising a tumour cell, or lysate thereof) of the subject, to thereby determine that the subject has increased likelihood of sensitivity to the therapy. In other embodiments, the methods comprise detecting a lower level of the co-localization relative to a control (e.g., a reference sample comprising a corresponding control SETDB1-expressing cell having an aberrant level of nuclear SETDB1, or lysate thereof).

In a related aspect, the present disclosure provides methods for determining likelihood of sensitivity to a therapy (e.g., immunotherapy) in a subject. These methods generally consist or consist essentially of detecting in a sample (e.g., a sample comprising a SETDB1-expressing cell such as a tumour cell, or lysate thereof) of the subject the absence of a complex comprising SETDB1 and a nuclear binding partner of SETDB1 (e.g., ATF7IP), or a level of the complex, which is indicative of a normal level of the complex and which correlates with an increased likelihood of sensitivity to the therapy, to thereby determine that the subject has increased likelihood of sensitivity to the therapy.

In some embodiments, the methods include detecting about the same level of the complex relative to a control (e.g., a reference sample comprising a corresponding normal or immunocompetent control SETDB1-expressing cell, or lysate thereof), to thereby determine that the subject has increased likelihood of sensitivity to the therapy.

In other embodiments, the methods comprise detecting a lower level of the complex relative to a control (e.g., a reference sample comprising a corresponding control SETDB1-expressing cell having an aberrant level of nuclear SETDB1, or lysate thereof), to thereby determine that the subject has increased likelihood of sensitivity to the therapy.

In another aspect, the present disclosure provides methods for analysing cellular localization of SETDB1 (e.g., in a tumour cell). These methods generally comprise, consist, or consist essentially of detecting the presence, absence or level of co-localization of SETDB1 with a nuclear binding partner of SETDB1 (e.g., ATF7IP, IMPα) in a cell, to thereby determine localization of SETDB1 in the cell. In some embodiments, presence of the co-localization is indicative of nuclear localization of SETDB1. In other embodiments, absence of the co-localization is indicative of cytoplasmic localization of SETDB1. In still other embodiments, the methods comprise detecting a normal level of the co-localization relative to a control (e.g., the level of co-localization in a corresponding normal or immunocompetent cell, or lysate thereof) which indicates that that there is a higher cytoplasmic localization of SETDB1 than nuclear localization of SETDB1. In still other embodiments, the methods comprise detecting a higher level of the co-localization relative to a control (e.g., the level of co-localization in a corresponding normal cell, or lysate thereof) which indicates that that there is a higher nuclear localization of SETDB1 than cytoplasmic localization of SETDB1. In representative examples of these embodiments, the co-localization is represented by a complex comprising SETDB1 and the nuclear binding partner of SETDB1.

Still another aspect of the present disclosure provides methods for stratifying a subject as a likely responder or non-responder to a therapy (e.g., immunotherapy). These methods generally comprise, consist or consist essentially of: analysing cellular localization of SETDB1 as broadly described above and elsewhere herein in a sample of the subject, to determine whether the subject has increased likelihood of sensitivity or resistance to the therapy, to thereby stratify the subject as a likely responder or non-responder to the therapy.

A further aspect of the present disclosure provides methods for managing treatment of a subject with a therapy (e.g., immunotherapy). These methods generally comprise, consist or consist essentially of: selecting a subject for treating with the therapy on the basis that the subject is a likely responder to the therapy, or selecting a subject for not treating with the therapy on the basis that the subject is a likely non-responder to the therapy and treating or not treating the subject with the therapy based on the selection, wherein the selection is based on the stratification method broadly described above and elsewhere herein.

In another aspect of the present disclosure, methods are provided for predicting treatment outcome of a subject with a therapy (e.g., immunotherapy). These methods generally comprise, consist or consist essentially of: analysing cellular localization of SETDB1 as broadly described above and elsewhere herein in a sample of the subject, to determine whether the subject has increased likelihood of sensitivity or resistance to the therapy, to thereby predict the treatment outcome for the subject. In some embodiments, the methods comprise detecting presence or a level of nuclear-localized SETDB1 relative to a control, which correlates with an increased likelihood of resistance to the therapy, as broadly described above and elsewhere herein, and predicting a negative treatment outcome. Suitably, the negative treatment outcome is greater disease severity or progressive disease. In other embodiments, the methods comprise detecting absence or a level of nuclear-localized SETDB1 relative to a control, which correlates with an increased likelihood of sensitivity to the therapy, as broadly described above and elsewhere herein, and predicting a positive treatment outcome. The positive treatment outcome may be selected from a partial or complete response to the therapy and stable disease. In any of these embodiments, the methods suitably further comprise predicting a clinical outcome for the subject based on the predicted treatment outcome. In non-limiting examples of this type, the subject is a cancer subject and the clinical outcome is selected from tumour response (TR), overall survival (OS), progression free survival (PFS), disease free survival, time to tumour recurrence (TTR), time to tumour progression (TTP), relative risk (RR), toxicity or side effect.

A further aspect of the present disclosure provides methods of monitoring a disease in a subject following treatment with a therapy. These methods generally comprise, consist or consist essentially of: obtaining a sample from the subject following treatment of the subject with the therapy (e.g., immunotherapy), wherein the sample comprises a SETDB1-expressing cell (e.g., a tumour cell); analysing cellular localization of SETDB1 as broadly described above and elsewhere herein in the sample, wherein a lower level of nuclear-localized SETDB1 relative to a control sample of the subject taken prior to the treatment is indicative of an increased clinical benefit of the therapy (e.g., lesser disease severity, delaying progression of the disease, reduced rate of disease progression, or absence or amelioration of the disease) to the subject and wherein a similar or higher level of nuclear-localized SETDB1 relative to the control sample is indicative of no or negligible clinical benefit of the therapy to the subject.

Yet a further aspect of the present disclosure provides methods for determining status of a disease in a subject. These methods generally comprise, consist or consist essentially of: analysing cellular localization of SETDB1 as broadly described above and elsewhere herein in a sample of the subject, to thereby determine the status of the disease in the subject, wherein presence or a level of nuclear-localized SETDB1, which correlates with an increased likelihood of resistance to a therapy, as broadly described above and elsewhere herein, indicates greater severity or progression of the disease in the subject and wherein absence or a level of nuclear-localized SETDB1, which correlates with an increased likelihood of sensitivity to a therapy, as broadly described above and elsewhere herein, indicates absence of the disease or lesser severity or progression of the disease in the subject.

Yet another aspect of the present disclosure provides kits for detecting location of SETDB1 in a cellular location (e.g., cytoplasm or nucleus) of a cell, for predicting the likelihood of response of a cell to a therapy (e.g., immunotherapy), for determining likelihood of resistance of a subject to a therapy (e.g., immunotherapy), for determining likelihood of sensitivity of a subject to a therapy (e.g., immunotherapy), for stratifying a subject as a likely responder or non-responder to a therapy (e.g., immunotherapy), for managing treatment of a subject with a therapy (e.g., immunotherapy), for monitoring a disease in a subject following treatment with a therapy, for determining the status of a disease in a subject and/or for determining the immune status of a subject. These kits generally comprise, consist or consist essentially of: a first antigen-binding molecule that binds specifically to SETDB1. In some embodiments, the kits comprise a second antigen-binding molecule that binds specifically to a nuclear binding partner of SETDB1 (e.g., ATF7IP, IMPα). In some embodiments, the kits further comprise a third antigen-binding molecule, which suitably comprises a detectable label, that binds to the first and second antigen-binding molecules.

Suitably, the kits further comprise instructional material for performing any one or more of the methods broadly described above and/or elsewhere herein.

Still another aspect of the present disclosure provides a complex comprising SETDB1 and a nuclear binding partner of SETDB1 (e.g., ATF7IP, IMPα), a first antigen-binding molecule that is bound specifically to SETDB1 of the complex and a second antigen-binding molecule bound to the nuclear binding partner of the complex. In some embodiments, the complex is located in a cell or lysate thereof. Suitably, the complex further comprises a third antigen-binding molecule, which is suitably detectably labelled, that binds to each of the first and second antigen-binding molecules of the complex.

In a further aspect, the present disclosure provides a cell or lysate thereof, comprising a complex broadly described above and elsewhere herein.

In certain embodiments of any of the above aspects, the therapy comprises an immunotherapy (e.g., an immune checkpoint inhibitor such as an antagonist antigen-binding molecule that binds specifically to an immune checkpoint molecule). In illustrative examples of this type, the immunotherapy comprises an antagonist antigen-binding molecule that binds specifically to PD-1.

### BRIEF DESCRIPTION OF THE FIGURES

FIG. 1. Interaction SETDB1/IMPA1 & SETDB1/ATF7IP: MSETC dose response. (A) Example fields of imaging are depicted with 10 μM scale bars in orange. Cells were permeabilized by incubating with 0.5% Triton X-100 for 20 mins were probed with a mouse anti-ATF7IP, rabbit anti-SETDB1 and goat anti-IMPα1 and visualized with a donkey anti-rabbit AF 568, anti-mouse 488, anti-goat 647. Cover slips were mounted on glass microscope slides with ProLong Clear Antifade reagent (Life Technologies). Protein targets were localized by ASI digital pathology. (B-E) Bar graphs show nuclear or cytoplasmic fluorescent intensity of SETDB1 with SEM, ratio of nuclear to cytoplasmic staining of SETDB1 (Fn/c). Graphs show the cytoplasmic PCC of SETDB1 and ATF7IP (B) and SETDB1 and IMPα1 (C). Graphs show the nuclear fluorescence intensity of SETDB1 (D) and ATF7IP (E).

FIG. 2. Effect of the SETDB1 prototype peptides on proliferation in MDA-MB-231 cells. MDA-MB-231 cells were treated with 047 and 047-1 for up to 72 hours. At 48 (A) and 72 hr (B) time points, media was removed and replaced with 100 μL/well of WST-1 cell proliferation reagent. Absorbance was recorded at 450 nm at 1 hr using a microplate spectrophotometer. RPMI-7951 melanoma cells were treated with 047 and 047-1 (C, D) for up to 72 hours. At both 48 and 72 hr time points, media was removed and replaced with 100 μl/well of WST-1 cell proliferation reagent. Absorbance was recorded at 450 nm at 1 hr using a microplate spectrophotometer.

FIG. 3. Effect of SETDB1 prototype peptides on CSV, SNAIL and SETDB1 protein expression in MDA-MB-231 cells. MDA-MB-231 cells were treated with vehicle alone or two different concentrations of peptide. Cells were stained with a panel targeting SETDB1, CSV and SNAIL, imaged (A) and quantified (B). Example images are depicted with 10 μM scale bars. (047=PEP1, 047-1=PEP2).

FIG. 4. Effect of the SETDB1 NLS linear peptide versus bicyclic peptide on proliferation in cancer cell lines. (A) Structural characterization of the IMPα1 (grey); SETDB1 (green); ATF7IP (blue) trimeric complex. (B) Electrophoretic mobility shift assay showing interaction between IMPα1 and MSETC. (C) Depicts table of epigenetic induced transcriptional pathways in mesenchymal breast cancer cell line. (D-E) MDA-MB-231 and MDA-MB-231 brain cells were treated with 047-1 or the MSETC bicyclic peptide for 72 hours. After incubation, media was removed and replaced with 100 μL/well of WST-1 cell proliferation reagent. Absorbance was recorded at 450 nm at 1 hr using a microplate spectrophotometer.

FIG. 5. (A, B) Proliferation assay of MSETC, TBAB-MSETC inhibition immunotherapy responsive line TNBC cell line MDA-MB-231 (A) and CT26 (B). Cells were treated with MSETC or MSETC-TBAB bicyclic peptide for 72 hours. After 72 hr, media was removed and replaced with 100 μL/cell of WST-1 cell proliferation reagent. Absorbance was recorded at 450 nm at 1 hr using a microplate spectrophotometer. (C)-(F) Lung cancer cell line LLC proliferation assay using TBAB-MSETC (C), MSETC (D), MSETC-D (E), and MSETD-D-R (F) inhibitors. After 72 hr, media was removed and replaced with 100 μL/well of WST-1 cell proliferation reagent. Absorbance was recorded at 450 nm at 1 hr using a microplate spectrophotometer.

FIG. 6. Bicyclic peptide treatment does not impact cell viability in healthy PBMC samples. Healthy PBMC were treated with various concentrations of MSETC (2.5-40 μM) overnight prior to staining with LIVE/DEAD™ Fixable Aqua Dead Cell Stain Kit (405 nm excitation). All samples were acquired on an LSR Fortessa cytometer and data were analyzed using FlowJo v10 software.

FIG. 7. MDA-MB-231 cells were treated with 10 mM MSETC. (A) Graphs of nuclear fluorescence intensity (NFI) of markers C-REL, G9A, PKCb1, and a custom antibody PDL1-PTM1. MDA-MB-231 cells were treated with MSETC or Control and were permeabilised before being probed with the antibodies specific for C-REL, G9A, PKCb1, and the custom antibody PDL1-PTM1. Cover slips were mounted on glass microscope slides with ProLong nucblue glass Antifade reagent (Life Technologies). Digital images were analysed using ImageJ software (ImageJ, NIH, Bethesda, MD, USA) and graphs represent the mean nuclear fluorescent intensity in the nucleus or the cytoplasm compartments with significant differences calculated as per One way Anova pairwise comparison. (B) H1299 cells treated with MSETC, General Importin-α1 inhibitor BIMAX or SETDB1 catalytic inhibitor MTH or vehicle were stained for off-target effects by high resolution imaging of DUOLINK cells stained PKC6 and IMPα1; LSD1 and IMPα1; G9A with and IMPα1; and ACE2 & IMPα1. H1299 cells were probed with the DUOLINK ligation assay.

FIG. 8. SETDB1 is cytoplasmic and has low expression in healthy PBMCs. Healthy donor PBMCs were isolated from liquid biopsies and stained for SETDB1. PBMC example fields of imaging are depicted with 10 μM scale bars in orange. Cells were permeabilized with 0.5% Triton X-100 for 20 mins and were probed with a rabbit anti-SETDB1 and visualized with anti-rabbit 568. Cover slips were mounted on glass microscope slides with ProLong Clear Antifade reagent (Life Technologies). Protein targets were localized by ASI digital pathology.

FIG. 9. SETDB1 is cytoplasmic with low expression in healthy donor tonsil tissues. Healthy tonsil tissue was stained using a BondRX automatic staining platform. Tissue example fields of imaging are depicted with 10 μM scale bars in orange. Tissue section was probed with a rabbit anti-SETDB1 and visualized with anti-rabbit 568. Cover slips were mounted on glass microscope slides with ProLong Clear Antifade reagent (Life Technologies). Protein targets were localized by ASI digital pathology.

FIG. 10. SETDB1 a novel epigenetic oncogene is enriched in NSCLC and other advanced cancers. (A) Proportion of TCGA cases with SETDB1 high- or low-level amplification (amp) BLCA, bladder urothelial carcinoma; BRCA, breast invasive carcinoma; CESC, cervical squamous cell carcinoma and endocervical adenocarcinoma; COAD, colon adenocarcinoma; ESCA, oesophageal carcinoma; GBM, glioblastoma multiforme; HNSC, head and neck squamous cell carcinoma; KIRC, kidney renal clear cell carcinoma; KIRP, kidney renal papillary cell carcinoma; LGG, brain lower grade glioma; LIHC, liver hepatocellular carcinoma; LUAD, lung adenocarcinoma; LUSC, lung squamous cell carcinoma; OV, ovarian serous cystadenocarcinoma; PAAD, pancreatic adenocarcinoma; PCPG, pheochromocytoma and paraganglioma; PRAD, prostate adenocarcinoma; SARC, sarcoma; STAD, stomach adenocarcinoma; SKCM, skin cutaneous melanoma; TGCT, testicular germ cell tumours; THCA, thyroid carcinoma; UCEC, uterine corpus endometrial carcinoma; UCS, uterine carcinosarcoma. (B) The level of SETB1 in 492 lung adenocarcinoma and 485 lung squamous carcinoma tissues in comparison to normal tissues (59 and 50 respectively) (http://egpia.cancer-pku.cn/). (C) Kaplan-Meiers survival analysis of SETDB1 expression in NSCLC patients with low and high tumor expression of SETDB1, calculated from (http://kmplotcomn/analysis/) with significant differences indicated.

FIG. 11. SETDB1 signature in CTCs from metastatic patient cohorts undergoing immunotherapy. Liquid biopsies are stratified into “Resistant” (A) and “Responder” (B) cohorts based on response to immunotherapy. CTCs were permeabilised by incubating with 0.5% Triton X-100 for 20 min and were probed with a rabbit anti-SETDB1, mouse anti-CSV and visualized with a donkey anti-rabbit AF 568, anti-mouse 488 secondary antibodies. Cover slips were mounted on glass microscope slides with ProLong nucblue clear Antifade reagent (Life Technologies). Protein targets were localised by confocal laser scanning microscopy. Single 0.5 μm sections were obtained u/sing an ASI Digital pathology system using 100× oil immersion lens running ASI software. The final image was obtained by averaging four sequential images of the same section. (C) Digital images were analysed using both ASI software for population dynamics and ImageJ software (ImageJ, NIH, Bethesda, MD, USA) to determine the ratio of nuclear to cytoplasmic staining (Fn/c), total cell counts and fluorescent intensity (expression of protein).

FIG. 12. Nuclear SETDB1 is enriched in immunotherapy-resistant patients and is not expressed in healthy nuclei. (A) Depicts PIE; Total CTCs per 10 mL or % CTC population CSV+SETDB1+CD45− in metastatic cancer liquid biopsies. (B) Representative digital pathology high-resolution immunofluorescence images of SETDB1 expression in immunotherapy-resistant and responsive Stage IV metastatic FFPE. (C) Graphs depict nuclear staining (NFI) of SETDB1, PCC (co-localisation) of SETDB1, ATF7IP and IMPα1. (D) Representative digital pathology high-resolution immunofluorescence images of SETDB1, CSV in patient liquid biopsies. Depicts Fn/c, NFI and CFI of SETDB1 in 10 resistant and responder Stage IV and Healthy donor (HD) liquid biopsies. (E) chart analysis of CD8+PD1+ T cells positive for terminally exhaustion marker EOMES^{AC} in responder or resistant patients with SETDB1 intensity. Rsp: Responsive, Rsi: Resistance.

FIG. 13. Increased SETDB1 nuclear expression in “resistant” patient cohort samples. (A) FFPE samples were processed on the BONDRX with the Opal staining kit targeting melanoma cancer marker [HMB45+M2-7C10+M2-9E3](ML) and SETDB1. Green is melanoma cancer marker [HMB45+M2-7C10+M2-9E3] and magenta is SETDB1. (B-D) Depicts bar graphs to show the ratio of nuclear to cytoplasmic staining of SETDB1 with SEM, integrated nuclear intensity or integrated cytoplasmic intensity. Significant differences are indicated, calculated by Kruskal-Wallis non-parametric test.

FIG. 14. Interaction SETDB1/IMPα1:MSETC dose response. MDA-MB-231 cells example fields of imaging are depicted with 10 μM scale bars in orange. Cells were treated with SETDB1 bicyclic inhibitor MSETC at concentrations 1.25 mM to 20 mM or vehicle control and were permeabilized by incubating with 0.5% Triton X-100 for 20 mins were probed with a rabbit anti-SETDB1 and mouse anti-IMPα1 and visualized with a donkey anti-rabbit AF 568, anti-mouse 488. Cover slips were mounted on glass microscope slides with ProLong Clear Antifade reagent (Life Technologies). Protein targets were localized by ASI digital pathology. (B) Bar graphs show nuclear or cytoplasmic fluorescent intensity of SETDB1 with SEM, ratio of nuclear to cytoplasmic staining of SETDB1 (Fn/c). (C) Graphs show the nuclear or cytoplasmic PCC of SETDB1/IMPα1. Significant differences are indicated calculated by Kruskal-Wallis non-parametric test. Red dotted line represents 50% inhibition.

FIG. 15. MDA-MB-231 cells were treated with MSETC; with concentrations ranging from 5 μM to 0.078 μM. This figure depicts high resolution imaging of DUOLINK cells stained with SETDB1 and IMPα1. MDA-MB-231 cells were treated with MSETC or control and were permeabilised and were probed with the DUOLINK ligation assay. Cover slips were mounted on glass microscope slides with ProLong nucblue glass Antifade reagent (Life Technologies). MSETC and IMPα1 DUOLINK Digital images were analysed using ImageJ software (ImageJ, NIH, Bethesda, MD, USA) and graphs represent the mean DOT Fluorescent Intensity with significant differences calculated as per Kruskal-Wallis one-way ANOVA. The IC50 is marked with a red dotted line.

FIG. 16. MSETC dual targeting inhibitor selective for SETDB1 and superior to catalytic inhibitor. (A) MDA-MB-231 cells were treated with 5 mM of MSETC, Catalytic SETDB1 inhibitor Mithramycin A (MTH) or vehicle control and were permeabilized by incubating with 0.5% Triton X-100 for 20 mins were probed with a rabbit anti-H3k27ac and mouse anti-H3k9me3 and visualized with a donkey anti-rabbit AF 568, anti-mouse 488. Cover slips were mounted on glass microscope slides with ProLong Clear Antifade reagent (Life Technologies). Protein targets were localized by ASI digital pathology. Bar graphs show nuclear fluorescence intensity of H3k9me3 and H3k27ac with significant differences indicated as calculated by Kruskal-Wallis non-parametric test. (B) Cells were treated with MSETC or vehicle. Cells were permeabilized and stained for H3.3Ser1p and H3k4me2 and digital pathology employed to analyze expression. Mann-Whitney was used to determine significance.

FIG. 17. (A) H1299 (lung cancer cells) or MDA-MB-231 cells were treated with MSETC (either MSETC or MSETC-D-isomer version (MSETC-D) or MSETC-D-isomer-retero-inverso (MSETC-D-R), General IMPα1 inhibitor BIMAX or SETDB1 catalytic inhibitor MTH. Data from high resolution imaging of DUOLINK cells are stained with SETDB1 a IMPα1. MDA-MB-231 cells or H1299 cells were probed with the DUOLINK ligation assay. Cover slips were mounted on glass microscope slides with ProLong nucblue glass Antifade reagent (Life Technologies). MSETC and IMPα1 DUOLINK Digital images were analysed using ImageJ software (ImageJ, NIH, Bethesda, MD, USA) and graphs represent the mean DOT Fluorescent Intensity with significant differences calculated as per Kruskal-Wallis one-way ANOVA.

FIG. 18. (A) H1299 cells (human non-small cell lung carcinoma cell line) were treated with 5 mM of MSETC, catalytic SETDB1 inhibitor Mithramycin A (MTH), or vehicle control and were permeabilized by incubating with 0.5% Triton X-100 for 20 mins were probed with a rabbit anti SETDB1 and mouse anti CSV and visualized with a donkey anti-rabbit AF 568, anti-mouse 488. Cover slips were mounted on glass microscope slides with ProLong Clear Antifade reagent (Life Technologies). Protein targets were localized by ASI digital pathology. Bar graphs show nuclear intensity of H3k9me3 and H3k27ac with significant differences are indicated calculated by Kruskal-Wallis non-parametric test. (B) H1299 cells were treated with 5 mM of MSETC or vehicle control and were permeabilized by incubating with 0.5% Triton X-100 for 20 mins were probed with a rabbit anti SETDB1 and mouse anti ATF7IP or goat anti IMPα1 and visualized with a donkey anti-rabbit AF 568, anti-mouse 488 and anti-goat 647. Cover slips were mounted on glass microscope slides with ProLong Clear Antifade reagent (Life Technologies). Protein targets were localized by ASI digital pathology. (C) Bar graphs show nuclear intensity or PCC of antibody pairs with significant differences are indicated calculated by Kruskal-Wallis non-parametric test.

FIG. 19. Increased expression of interferon signaling genes in MCF-7 cells following treatment with the bicyclic peptide. Real-time qPCR analysis of MCF-7 cells treated with bicyclic peptide (5 μM) for 24 hours prior to stimulation with phorbol 12-myristate 13-acetate (PMA; 20 ng/mL; Sigma) or Poly (I:C) (500 ng/mL; Sigma) for 48 hours. Cts were converted to arbitrary copy numbers and normalized to the geomean of the house-keeping genes ACTB and PPIA. ****p<0.0001, *** p<0.001, **p<0.01, *p<0.05, unpaired t test.

FIG. 20. Increased expression of Viral mimicry and immunogenicity/immune visibility genes in epithelial & mesenchymal MCF-7 cells following treatment with the bicyclic peptide. Real-time qPCR analysis of MCF-7 cells treated with bicyclic peptide (5 μM) for 24 hours prior to stimulation with phorbol 12-myristate 13-acetate (PMA; 20 ng/mL; Sigma) to induce a mesenchymal signature for 48 hours. Cts were converted to arbitrary copy numbers and normalized to the geomean of the house-keeping genes ACTB and PPIA. ****p<0.0001, *** p<0.001, **p<0.01, *p<0.05, unpaired t test.

FIG. 21. Bicyclic peptide (MSETC)+/−αPD1 therapy in the 4T1 model of metastatic breast cancer. (A) Treatment regime using the Balb/c 4T1 breast cancer model. (B) Mice body weight (g) over 20 day treatment period. (C) Tumour volumes of mice treated with vehicle (saline) or MSETC (20 mg/kg) in conjunction with αPD1 or isotype control (10 mg/kg) (n=4-5/group). (D) Tumour volumes of individual mice at day 20 post inoculation (data also represented in C. * p<0.05, ** p<0.01, Tukey's post test. (E) Representative images of tumours harvested at day 20 post inoculation.

FIG. 22. MSETC+/−αPD1 therapy does not alter lung, liver or spleen weights in the 4T1 model of metastatic breast cancer. (A) Representative images of organs harvested at day 20 post inoculation. (B) Final lung, liver and spleen weights at day 20 post inoculation. * p<0.05, Tukey's posttest.

FIG. 23. MSETC and αPD1 combination therapy reduces lung metastasis in the 4T1 model of metastatic breast cancer. (A) Representative images of lungs fixed in Bouin's solution. (B) Lung nodule counts in mice treated with vehicle (saline) or MSETC (20 mg/kg) in conjunction with αPD1 or isotype control (10 mg/kg) (n=4-5/group) at day 20 post inoculation. * p<0.05, one-wayANOVA.

FIG. 24. MSETC and αPD1 combination therapy reduces primary tumour CSV expression in metastatic breast cancer. (A) Representative images of FFPE primary tumour samples which were processed on the BONDRX with the Opal staining kit targeting Cytokeratin, CSV or SETB1. (B) Quantification of CSV, Cytokeratin or SETDB1 fluorescence intensity (FI). (C-D) Quantification of Cytokeratin (CYT), CSV and nuclear SETDB1 (nSETDB1) positive cells. Significant differences are indicated calculated by Kruskal-Wallis non-parametric test.

FIG. 25. MSETC and αPD1 combination therapy reduces lung CSV expression in metastatic breast cancer. (A) Representative images of FFPE lung samples which were processed on the BONDRX with the Opal staining kit targeting Cytokeratin, CSV or SETB1. (B) Quantification of CSV, Cytokeratin or SETDB1 fluorescence intensity (FI). (C-D) Quantification of Cytokeratin (CYT), CSV and nuclear SETDB1 (nSETDB1) positive cells. Significant differences are indicated calculated by Kruskal-Wallis non-parametric test.

FIG. 26. MSETC and αPD1 combination therapy increases primary tumour T_{RM}, T_{EM}, T_{CM} and T effector cell populations in metastatic breast cancer. (A) Representative images of FFPE primary tumour samples which were processed on the BONDRX with the Opal staining kit targeting either CD44, CD103, CD69, CD62L or CD8. Stained sections were analysed for populations of Tissue resident memory (B), Effector memory (C), Central memory (D) or Effector (E) T cells. Significant differences are indicated calculated by Kruskal-Wallis non-parametric test. 4-5 tissue sections were analysed for each group with >500 cells counted per a section.

FIG. 27. MSETC reprograms histone code in 4T1 tumour model. (A) Primary tumour samples from a 4T1 tumour model treated with as depicted was on the BONDRX with the Opal staining kit targeting CSV (tumour marker), H3k27ac, and H3k9me3. (B) Graphs depict the nuclear fluorescence intensity (NFI) of H3k27ac and H3k9me3. Significant differences are indicated calculated by Kruskal-Wallis non-parametric test.

FIG. 28. C-DR (5 mg/kg) vs MSETC (15 mg/kg) administration in combination with anti-PD1 reduces tumour burden in the 4T1 syngeneic tumour model. (A) Treatment regime using the Balb/c 4T1 breast cancer model. (B) Mice body weight (g) prior to Day 9 cull. Data presented as mean±SEM. (C) Tumour volumes of mice treated with MSETC-DR (5 mg/kg) versus MSETC (15 mg/kg) in conjunction with αPD1 (10 mg/kg) (n=5/group). Data presented as fold change versus Day 0. a; **p<0.01 MSETC and MSETC-DR versus αPD1, b; **p<0.01 MSETC and MSETC-DR versus αPD1, c: **p<0.01 and *p<0.05 MSETC and MSETC-DR respectively versus αPD1, d; **p<0.01 MSETC and MSETC-DR versus αPD1, Two-way ANOVA, Dunnett's post test. (D) Representative images of tumours harvested at Day 9. (E) Liver, spleen and lung weights (mg) at Day 9.

FIG. 29. The effect of MSETC and MSETC-DR on cell migration in the MCF-7 PMA/TGFβ inducible model. (A) Wound healing (scratch assay) analysis of the impact of MSETC and MSETC-DR on cell migration over a 24 hr period. Black line: Water control, Blue line: MSETC (20 μM), Orange line: MSETC-DR (20 μM). (B) As represented in (A), graphical representation of relative wound density (%) at 24 hr timepoint. **p<0.01, **** p<0.0001, one-way ANOVA, Dunnett's post test (n=6/group).

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” as used herein refers to the usual error range for the respective value readily known to the skilled person in this technical field. Reference to “about” a value or parameter herein includes (and describes) embodiments that are directed to that value or parameter per se.

The term “antagonist” or “inhibitor” refers to a substance that prevents, blocks, inhibits, neutralizes, or reduces a biological activity or effect of another molecule, such as an enzyme or receptor. The term “antagonist antibody” refers to an antibody that binds to a target and prevents or reduces the biological effect of that target. In some embodiments, the term can denote an antibody that prevents the target, e.g., PD-1, to which it is bound from performing a biological function.

The term “antibody” herein is used in the broadest sense and specifically covers monoclonal antibodies (including full length monoclonal antibodies), polyclonal antibodies, chimeric antibodies, humanized antibodies, human antibodies, multispecific antibodies (e.g., bispecific antibodies), and antibody fragments so long as they exhibit the desired biological activity.

The “amount” or “level” of a biomarker is a detectable level in a sample. These can be measured by methods known to one skilled in the art and also disclosed herein. The expression level or amount of biomarker assessed can be used to determine the response to treatment.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimetres, preferably from within about 0.5 to about 5 centimetres. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, SETDB1 bicyclic peptide mimetics such as peptides, polypeptides and proteins as well as compositions comprising them and genetic molecules such as RNA, DNA and mimetics and chemical analogs thereof as well as cellular agents.

As used herein, “and/or” refers to and encompasses any and all possible combinations of one or more of the associated listed items, as well as the lack of combinations when interpreted in the alternative (or).

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 mM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

The term “biomarker” as used herein refers to an indicator, e.g., predictive, diagnostic, and/or prognostic, which can be detected in a sample.

The terms “cancer” and “cancerous” refer to or describe the physiological condition in subjects that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumours), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In specific embodiments, the cancer is melanoma or lung cancer, suitably metastatic melanoma or metastatic lung cancer.

“Chemotherapeutic agent” includes compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); ad renocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gII and calicheamicin w1I (Angew Chem. Inti. Ed. Engl. 1994 33:183-186); dynemicin, including dynemicin A; bisphosphonates, such as clodronate; an esperamicin; as well as neocarzi nostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also include (i) anti-hormonal agents that act to regulate or inhibit hormone action on tumours such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signalling pathways implicated in aberrant cell proliferation, such as, for example, PKC-a, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idee), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, peefusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁I antibody genetically modified to recognize interleukin-12 p40 protein.

Chemotherapeutic agent also includes “EGFR inhibitors,” which refers to compounds that bind to or otherwise interact directly with EGFR and prevent or reduce its signalling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-a for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3-fluorophenyl) methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules {e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO1999/09016 (American Cyanamid); WO 1998/43960 (American Cyanamid); WO 1997/38983 (Warner Lambert); WO1999/06378 (Warner Lambert); WO1999/06396 (Warner Lambert); WO1996/30347 (Pfizer, Inc); WO1996/33978 (Zeneca); WO1996/3397 (Zeneca) and WO1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumour necrosis factor a (TNF-α) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T-cell costimulation blockers such as abatacept (Orencia), Interleukin 6 (IL-6) blockers such as tocilizumab (ACTEMERA®); Interleukin 13 (IL-13) blockers such as lebrikizumab; Interferon a (IFN) blockers such as Rontalizumab; Beta 7 integrin blockers such as rhuMAb Beta7; IgE pathway blockers such as Anti-Mi prime; Secreted homotrimeric LTa3 and membrane bound heterotrimer LTal/82 blockers such as Anti-lymphotoxin a (LTa); radioactive isotopes (e.g., At²¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhea, metastatic bone pain, headache and migraine, postoperative pain, mild-to-moderate pain due to inflammation and tissue injury, pyrexia, ileus, and renal colic.

As used herein, the term “co-localization” or “co-localized” refers two or more molecules having identical or overlapping localization in the cell. Co-localization of molecules and proteins can be detected using any suitable method known in the art, including for example, fluorescent microscopy in fixed or living cells. For example, SETDB1 and a SETDB1 nuclear binding partner (e.g., ATF7IP, IMPα) can be co-localized in cells using fluorescently-labelled anti-SETDB1 and anti-nuclear binding partner primary antibodies and optionally one or more secondary antibodies. Methods of co-localization of cellular molecules are well known.

The terms “cell proliferative disorder”, “proliferative disorder” and “hyperproliferative disorder” are used interchangeably herein to refer to disorders that are associated with some degree of abnormal cell proliferation. In some embodiments, the cell proliferative disorder is cancer. In some embodiments, the cell proliferative disorder is a tumour, including a solid tumour.

As used herein, a “companion diagnostic” refers to a diagnostic method and or reagent that is used to identify subjects susceptible to treatment with a particular treatment or to monitor treatment and/or to identify an effective dosage for a subject or sub-group or other group of subjects. For purposes herein, a companion diagnostic refers to reagents, such as a reagent for detecting or measuring SETDB1 cellular localization (e.g., as described herein) in a sample. The companion diagnostic refers to the reagents and also to the test(s) that is/are performed with the reagent.

As used herein, the term “complex” refers to an assemblage or aggregate of molecules (e.g., peptides, polypeptides, etc.) in direct and/or indirect contact with one another. In specific embodiments, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favoured (e.g., compared to a non-aggregated, or non-complexed, state of its component molecules). The term “polypeptide complex” or “protein complex,” as used herein, refers to a trimer, tetramer, pentamer, hexamer, heptamer, octamer, nonamer, decamer, undecamer, dodecamer, or higher order oligomer. In specific embodiments, the polypeptide complexes are formed by self-assembly of SETDB1 and a nuclear binding partner of SETDB1 (e.g., ATF7IP, IMPα).

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “correlate” or “correlating” refer to determining a relationship between one type of data with another or with a state (e.g., response to therapy). In some embodiments, “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polypeptide analysis or protocol, one may use the results of the polypeptide expression or cellular localization analysis or protocol to determine whether a specific therapeutic regimen should be performed.

By “corresponds to” or “corresponding to” is meant an amino acid sequence that displays substantial sequence similarity or identity to a reference amino acid sequence. In general the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

By “derivative” is meant a molecule, such as a polypeptide, that has been derived from the basic molecule by modification, for example by conjugation or complexing with other chemical moieties or by post-translational modification techniques as would be understood in the art. The term “derivative” also includes within its scope alterations that have been made to a parent sequence including additions or deletions that provide for functionally equivalent molecules.

As used herein, the term “dosage unit form” refers to physically discrete units suited as unitary dosages for the subject to be treated, each unit containing a predetermined quantity of active material calculated to produce the desired therapeutic effect in association with the required pharmaceutically acceptable vehicle.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of a cancer or tumour, an effective amount of the drug may have the effect in reducing the number of cancer cells; reducing the tumour size; inhibiting (i.e., slow to some extent or desirably stop) cancer cell infiltration into peripheral organs; inhibit (i.e., slow to some extent and desirably stop) tumour metastasis; inhibiting to some extent tumour growth; and/or relieving to some extent one or more of the symptoms associated with the cancer or tumour. In the case of an infection, an effective amount of the drug may have the effect in reducing pathogen (bacterium, virus, etc.) titers in the circulation or tissue; reducing the number of pathogen infected cells; inhibiting (i.e., slow to some extent or desirably stop) pathogen infection of organs; inhibit (i.e., slow to some extent and desirably stop) pathogen growth; and/or relieving to some extent one or more of the symptoms associated with the infection. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer. A patient who “does not have an effective response” to treatment refers to a patient who does not have any one of extending survival (including overall survival and progression free survival); resulting in an objective response (including a complete response or a partial response); or improving signs or symptoms of cancer.

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only. The term “expression” is also used herein to refer to the presence of a protein or molecule in a particular location and, thus, may be used interchangeably with “localization”.

The term “exhaustion” and its grammatical equivalents refer to T-cell exhaustion as a state of T-cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T-cells. Exhaustion prevents optimal control of infection and tumours. Exhaustion can result from both extrinsic negative regulatory pathways (e.g., immunoregulatory cytokines) as well as cell intrinsic negative regulatory (costimulatory) pathways (PD-1, B7-H3, B7-H4, etc.).

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

As used herein, the term “higher” with reference to a biomarker or biomarker complex measurement refers to a statistically significant and measurable difference in the level of a biomarker or biomarker complex measurement compared to the level of another biomarker or biomarker complex or to a control level where the biomarker or biomarker complex measurement is greater than the level of the other biomarker or biomarker complex or the control level. The difference is preferably at least about 10%, or at least about 20%, or of at least about 30%, or of at least about 40%, or at least about 50%.

The term “host cell” includes an individual cell or cell culture which can be or has been a recipient of any recombinant vector(s) or isolated polynucleotide of the invention. Host cells include progeny of a single host cell and the progeny may not necessarily be completely identical (in morphology or in total DNA complement) to the original parent cell due to natural, accidental or deliberate mutation and/or change. A host cell includes cells transfected or infected in vivo or in vitro with a recombinant vector or a polynucleotide of the invention. A host cell which comprises a recombinant vector of the invention is a recombinant host cell.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The term “immunotherapy” refers to any therapy in which one or more components of a human's or animal's immune system is deliberately modulated in order to directly or indirectly achieve some therapeutic benefit, including systemic and/or local effects, and preventative and/or curative effects. Immunotherapy can involve administering one or more immunotherapeutic agents, either alone or in any combination, to a human or animal subject by any route (e.g., orally, intravenously, dermally, by injection, by inhalation, etc.), whether systemically, locally or both. Immunotherapy can involve provoking, increasing, decreasing, halting, preventing, blocking or otherwise modulating the production of cytokines, and/or activating or deactivating cytokines or immune cells, and/or modulating the levels of immune cells, and/or delivering one or more therapeutic or diagnostic substances to a particular location in the body or to a particular type of cell or tissue, and/or destroying particular cells or tissue. Immunotherapy can be used to achieve local effects, systemic effects or a combination of both.

The term “immunotherapeutic agent” as used herein refers to any agent, compound, or biologic that indirectly or directly restores, enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively, the immunotherapeutic treatment may consist of administering the subject with an amount of immune cells (T-cells, NK, cells, DCs, B-cells, etc.). Immunotherapeutic agents can be non-specific, i.e., boost the immune system generally so that the human body becomes more effective in fighting the growth and/or spread of cancer cells, or they can be specific, i.e., targeted to the cancer cells themselves. Immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g., cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present disclosure include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behavior and/or increasing their production of antigens thus making the cancer cells easier for the immune system to recognize and destroy. IFNs can also act indirectly on cancer cells, for example, by slowing down angiogenesis, boosting the immune system and/or stimulating natural killer (NK) cells, T-cells and macrophages. Recombinant IFN-alpha is available commercially as Roferon (Roche Pharmaceuticals) and Intron A (Schering Corporation). Interleukins contemplated by the present disclosure include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present disclosure. Colony-stimulating factors (CSFs) contemplated by the present disclosure include granulocyte colony stimulating factor (G-CSF or filgrastim), granulocyte-macrophage colony stimulating factor (GM-CSF or sargramostim) and erythropoietin (epoetin alfa, darbopoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Aranesp (erythropoietin). In addition to having specific or non-specific targets, immunotherapeutic agents can be active, i.e., stimulate the body's own immune response including humoral and cellular immune responses, or they can be passive, i.e., comprise immune system components such as antibodies, effector immune cells, antigen-presenting cells etc. that were generated external to the body. In specific embodiments, passive immunotherapy involves the use of one or more monoclonal antibodies that are specific for a particular antigen found on the surface of a cancer cell or immune cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents include, but are not limited to, alemtuzumab (LEMTRADA®), bevacizumab (AVASTIN®), cetuximab (ERBITUX®), panitumumab (VECTIBIX®), pertuzumab (OMNITARG®, 2C4), trastuzumab (HERCEPTIN®), tositumomab (Bexxar®), abciximab (REOPRO®), adalimumab (HUMIRA®), apolizumab, aselizumab, atlizumab, bapineuzumab, basiliximab (SIMULECT®), bavituximab, belimumab (BENLYSTA®) briankinumab, canakinumab (ILARIS®), cedelizumab, certolizumab pegol (CIMZIA®), cidfusituzumab, cidtuzumab, cixutumumab, clazakizumab, crenezumab, daclizumab (ZENAPAX®), dalotuzumab, denosumab (PROLIA®, XGEVA®), eculizumab (SOLIRIS®), efalizumab, epratuzumab, erlizumab, felvizumab, fontolizumab, golimumab (SIMPONI®), ipilimumab, imgatuzumab, infliximab (REMICADE®), labetuzumab, lebrikizumab, lexatumumab, lintuzumab, lucatumumab, lulizumab pegol, lumretuzumab, mapatumumab, matuzumab, mepolizumab, mogamulizumab, motavizumab, motovizumab, muronomab, natalizumab (TYSABRI®), necitumumab (PORTRAZZA®), nimotuzumab (THERACIM®), nolovizumab, numavizumab, olokizumab, omalizumab (XOLAIR®), onartuzumab (also known as MetMAb), palivizumab (SYNAGIS®), pascolizumab, pecfusituzumab, pectuzumab, pembrolizumab (KEYTRUDA®), pexelizumab, priliximab, ralvizumab, ranibizumab, (LUCENTIS®), reslivizumab, reslizumab, resyvizumab, robatumumab, rontalizumab, rovelizumab, ruplizmnab, sarilumab, secukinumab, seribantumab, sifalimumab, sibrotuzumab, siltuximab (SYLVANT®) siplizumab, sontuzumab, tadocizumab, talizumab, tefibazumab, tocilizumab (ACTEMRA®), toralizumab, tucusituzumab, umavizmab, urtoxazumab, ustekinumab (STELARA®), vedolizumab (ENTYVIO®), visilizumab, zanolimumab, zalutumumab. In specific embodiments, the immunotherapy comprises a T-cell therapy, representative examples of which include adoptive T-cell therapy, tumour-infiltrating lymphocyte (TIL) immunotherapy, autologous cell therapy, engineered autologous cell therapy, and allogeneic T-cell transplantation. Non-limiting examples of T-cell therapies are described in U.S. Patent Publication Nos. 2014/0154228 and 2002/0006409, U.S. Pat. No. 5,728,388, and International Publication No. WO 2008/081035. The T-cells of the immunotherapy can come from any source known in the art. For example, T-cells can be differentiated in vitro from a hematopoietic stem cell population, or T-cells can be obtained from a subject. T-cells can be obtained from, e.g., peripheral blood mononuclear cells, bone marrow, lymph node tissue, cord blood, thymus tissue, tissue from a site of infection, ascites, pleural effusion, spleen tissue, and tumours, combination thereof. Alternatively, or in addition, the T-cells can be derived from one or more T-cell lines available in the art. T-cells can also be obtained from a unit of blood collected from a subject using any number of techniques known to the skilled artisan, such as FICOLL™ separation and/or apheresis. Additional methods of isolating T-cells for T-cell therapy are disclosed in U.S. Patent Publication No. 2013/0287748.

As used herein, the term “increase” or “increased” with reference to a biomarker or biomarker complex level refers to a statistically significant and measurable increase in the biomarker or biomarker complex level compared to the level of another biomarker or biomarker complex or to a control level. The increase is preferably an increase of at least about 10%, or an increase of at least about 20%, or an increase of at least about 30%, or an increase of at least about 40%, or an increase of at least about 50%.

The term “inhibitor” as used herein refers to an agent that decreases or inhibits at least one function or biological activity of a target molecule.

As used herein, “instructional material” includes a publication, a recording, a diagram, or any other medium of expression which can be used to communicate the usefulness of the compositions and methods of the disclosure. The instructional material of the kit of the disclosure may, for example, be affixed to a container which contains the therapeutic or diagnostic agents of the disclosure or be shipped together with a container which contains the therapeutic or diagnostic and/or prognostic agents of the disclosure.

As used herein, the term “isolated” refers to material that is substantially or essentially free from components that normally accompany it in its native state. For example, an “isolated peptide” refers to in vitro isolation and/or purification of a SETDB1 peptide mimetic from its natural cellular environment and from association with other components of the cell. “Substantially free” means that a preparation of peptide is at least 10, 15, 20, 25, 30, 35, 40, 45, 50, 55, 60, 65, 70, 75, 80, 85, 90, 91, 92, 93, 94, 95, 96, 97, 98 or 99% pure. In a preferred embodiment, the preparation of peptide has less than about 30, 25, 20, 15, 10, 9, 8, 7, 6, 5, 4, 3, 2 or 1% (by dry weight) of molecules that are not the subject of this invention (also referred to herein as “contaminating molecules”). When a peptide is recombinantly produced, it is also desirably substantially free of culture medium, i.e., culture medium represents less than about 20, 15, 10, 5, 4, 3, 2 or 1% of the volume of the preparation. The invention includes isolated or purified preparations of at least 0.01, 0.1, 1.0, and 10 milligrams in dry weight.

The term “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

As used herein, the term “localize” and its grammatical equivalents mean to accumulate in, or be restricted to, a specific or limited space or area, for example a specific cell, tissue, organelle, or intracellular region such as a cellular component (e.g., nucleus, cytoplasm, nuclear membrane, plasma membrane, etc.).

As used herein, the term “lower” with reference to a biomarker or biomarker complex measurement refers to a statistically significant and measurable difference in the level of a biomarker or biomarker complex measurement compared to the level of another biomarker or biomarker complex or to a control level where the biomarker or biomarker complex measurement is less than the level of the other biomarker or biomarker complex or the control level. The difference is preferably at least about 10%, or at least about 20%, or of at least about 30%, or of at least about 40%, or at least about 50%.

By “obtained” is meant to come into possession. Samples so obtained include, for example, polypeptide extracts isolated or derived from a particular source, including cell lysates. For instance, the extract may be isolated directly from a biological fluid or tissue of a subject.

As used herein, the terms “peptide”, “polypeptide” and “protein” are used interchangeably to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject peptides are particularly useful. Included within the definition are, for example, peptides containing one or more analogues of an amino acid including, for example, unnatural amino acids or polypeptides with substituted linkages.

The term “pharmaceutical composition” or “pharmaceutical formulation” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

As used herein, the terms “prevent”, “prevented” or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

The terms “reduce”, “inhibit”, “suppress”, “decrease”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, fatigue, etc. In another embodiment, the reduction may be determined objectively, for example when the number of CSCs and/or non-CSC tumour cells in a sample from a patient is lower than in an earlier sample from the patient. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

As used herein, a cancer subject who has been treated with a therapy is considered to “respond”, have a “response”, have “a positive response” or be “responsive” to the therapy if the subject shows evidence of an anti-cancer effect according to an art-accepted set of objective criteria or reasonable modification thereof, including a clinically significant benefit, such as the prevention, or reduction of severity, of symptoms, or a slowing of the progression of the cancer. It will be understood that the aforementioned terms may also be used in regard to the cancer. A variety of different objective criteria for assessing the effect of anti-cancer treatments on cancers are known in the art. The World Health Organization (WHO) criteria (Miller, A B, et al., Cancer 1981; 47(1):207-14) and modified versions thereof, the Response Evaluation Criteria in Solid Tumours (RECIST) (Therasse P, et al., J Natl Cancer Inst 2000; 92:205-16), and revised version thereof (Eisenhauer E A, New response evaluation criteria in solid tumours: revised RECIST guideline (version 1.1). Eur J Cancer 2009; 45(2):228-47) are sets of objective criteria, based on imaging measurements of the size and number of tumour lesions and detection of new lesions, e.g., from computed tomography (CT), magnetic resonance imaging (MRI), or conventional radiographs. Dimensions of selected lesions (referred to as target lesions) are used to calculate the change in tumour burden between images from different time points. The calculated response is then categorized as complete response (CR), partial response (PR), stable disease (SD), or progressive disease (PD). CR is complete disappearance of tumour (−100%), and PD is an increase of about 20%-25% or greater (depending on the particular criteria) and/or the appearance of new lesions. PR is a significant reduction (of at least about 30%) in size of tumour lesions (without emergence of new lesions) but less than a complete response. SD is in between PR and PD. (See Tables 1 and 2 for details.) These criteria are widely used as a primary endpoint in Phase II trials evaluating the efficacy of anti-cancer agents, e.g., as a surrogate for overall survival. However, anatomic imaging alone using WHO, RECIST, and RECIST 1.1 criteria were designed to detect early effects of cytotoxic agents and have certain limitations, particularly in assessing the activity of newer cancer therapies that stabilize disease. Clinical response patterns in subjects treated with immunotherapeutic anti-cancer agents or molecularly targeted anti-cancer agents may extend beyond those of cytotoxic agents and can manifest after an initial increase in tumour burden or the appearance of new lesions. For example, meaningful tumour responses to immune checkpoint inhibitor may occur after a delay, in some cases following WHO- or RECIST-defined PD. Criteria designated immune-related response criteria (irRC) were defined in an attempt to capture additional favourable response patterns observed with immune therapies (Wolchok, J D, et al.).

Guidelines for the evaluation of immune therapy activity in solid tumours: immune-related response criteria. Clin. Care Res. 15, 7412-7420.). Four patterns associated with favourable survival were identified, i.e., decreased baseline lesions without new lesions; durable stable disease; initial increase in total tumour burden but eventual response; and a reduction in total tumour burden during or after the appearance of new lesion(s), of which the latter two are distinct from the response patterns considered favorable according to WHO or RECIST criteria. The irRC include criteria for complete response (irCR), partial response (irPR), stable disease (irSD), and progressive disease (irPD). Among other things, the irRC incorporates measurable new lesions into “total tumour burden” and compares this variable to baseline measurements rather than assuming that new lesions necessarily represent progressive disease. In summary, according to the immune-related response criteria, irCR is complete disappearance of all lesions whether measurable or not, and no new lesions; irPR is a decrease in tumour burden 50% relative to baseline; irSD is disease not meeting criteria for irCR or irPR, in absence of it progressive disease (irPD); irPD is an increase in tumour burden 25% relative to nadir (the minimum recorded tumour burden) (Wolchok, supra). irCR, irPR and irPD require confirmation by a repeat, consecutive assessment at least 4 weeks from the date of first documentation. irCR, irPR, and irSD include all subjects with CR, PR, or SD by WHO criteria as well as those subjects that shift to these irRC categories from WHO PD. However, some subjects who would be classified as having PD according to WHO or RECIST criteria are instead classified as having PR or SD according to the irRC, identifying them as likely to have favorable survival. The irRC are applicable to immune checkpoint inhibitors and other immunotherapeutic agents. One of ordinary skill in the art will appreciate that additional response criteria are known in the art, which take into consideration various factors such as changes in the degree of tumour arterial enhancement and/or tumour density as indicators of tumour viable tissue, with decreased arterial enhancement and decreased tumour density being indicators of reduced viable tumour tissue (e.g., due to tumour necrosis). For example, modified RECIST criteria (mRECIST) take into consideration changes in the degree of tumour arterial enhancement (Lencioni R and Llovet J M. Semin Liver Dis 30: 52-60, 2010). Choi criteria and modified Choi criteria take into consideration decrease in tumour density on CT. Choi H, et al., J Clin Oncol 25: 1753-1759, 2007; Nathan P D, et al., Cancer Biol Ther 9: 15-19, 2010; Smith A D, et al., Am J Roentgenol 194: 157-165, 2010. Such criteria may be particularly useful in certain cancer types and/or with certain classes of therapeutic agents. For example, changes in tumour size can be minimal in tumours such as lymphomas, sarcoma, hepatomas, mesothelioma, and gastrointestinal stromal tumour despite effective treatment. CT tumour density, contrast enhancement, or MRI characteristics appear more informative than size. In certain embodiments functional imaging, e.g., using positron emission tomography (PET) may be used. For example, PET response criteria in solid tumours (PERCIST) may be used, in which the treatment response is evaluated by metabolic changes assessed with (18)F-FDG PET imaging, with decreased uptake of the tracer being indicative of (Wahl R L, et al., J Nucl Med 2009; 50, Suppl 1:122S-50S). It will also be understood that response criteria developed for various specific cancer types such as melanoma, breast cancer and lung cancer, are known in the art. By contrast, a cancer subject who has been treated with a therapy is considered “not to respond”, “to lack a response”, to have “a negative response” or be “non-responsive” to the therapy if the therapy provides no clinically significant benefit, such as the prevention, or reduction of severity, of symptoms, or increases the rate of progression of the cancer.

For purposes of the present disclosure, a cancer subject treated with an immunotherapy (e.g., an immune checkpoint inhibitor) as monotherapy or in combination with one or more other active agents (e.g., a complement inhibitor, an additional anti-cancer agent, or both) is considered to “respond”, have a “response”, or be “responsive” to the treatment if the subject has a complete response, partial response, or stable disease according at least to the immune-related response criteria. (The cancer subject may also respond according to RECIST, RECIST 1.1, WHO, and/or other criteria such as those mentioned above). Likewise, the cancer in such cases is said to “respond”, be “responsive”, or be “sensitive” to the treatment. The cancer subject is considered to “not respond”, not have a “response”, or to be “non-responsive” to the treatment if the subject has progressive disease according to the immune-related response criteria. The cancer subject may also not respond according to RECIST, RECIST 1.1, WHO, and/or other criteria such as those mentioned above. Likewise, the cancer in such cases said to “not respond”, or to be “nonresponsive”, “insensitive” or “resistant” to the treatment. A cancer is also considered to have become resistant to treatment if it initially responds but the subject subsequently exhibits progressive disease in the presence of treatment. Thus, for example, for methods and products described herein that relate to response to treatment for cancer (e.g., methods of predicting likelihood of response, methods of classifying subjects according to predicted response, methods of increasing the likelihood of response) a response is defined as irCR, irPR, or irSD, and lack of response is defined as irPD unless otherwise specified. In certain embodiments any useful response criteria may be specified. The response criteria may have been shown to correlate with a benefit such as increased overall survival or other clinically significant benefit. It will be appreciated that refinements or revisions of existing response criteria that, e.g., encompass additional favourable patterns of clinical activity (e.g., correlating with increased overall survival) applicable to immune checkpoint inhibitors or are otherwise useful may be developed in the future. In certain embodiments any such response criteria may be specified for use in methods described herein.

As used herein, the terms “salts” and “prodrugs” include any pharmaceutically acceptable salt, ester, hydrate or any other compound which, upon administration to the recipient, is capable of providing (directly or indirectly) a SETDB1 peptide mimetic of the invention, or an active metabolite or residue thereof. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. However, it will be appreciated that non-pharmaceutically acceptable salts also fall within the scope of the invention since these may be useful in the preparation of pharmaceutically acceptable salts. The preparation of salts and prodrugs can be carried out by methods known in the art. For example, metal salts can be prepared by reaction of a compound of the invention with a metal hydroxide. An acid salt can be prepared by reacting an appropriate acid with a SETDB1 peptide mimetic of the invention.]

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., feces), tears, sweat, sebum, nipple aspirate, ductal lavage, tumour exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises leukocytes including peripheral blood mononuclear cells (PBMC).

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumour). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

The term “scaffold” or “molecular scaffold” as used herein refers to a chemical moiety that is bonded to the peptide at the alkylamino linkages and thioether linkage (when cysteine is present) in the compositions of the invention. The term “scaffold molecule” or “molecular scaffold molecule” as used herein refers to a molecule that is capable of being reacted with a peptide or peptide ligand to form the derivatives of the invention having alkylamino and, in certain embodiments, also thioether bonds. Thus, the scaffold molecule has the same structure as the scaffold moiety except that respective reactive groups (such as leaving groups) of the molecule are replaced by alkylamino and thioether bonds to the peptide in the scaffold moiety.

“SETDB1” refers to the protein also known as “SET Domain Bifurcated Histone Lysine Methyltransferase 1”, “ESET”, and “KMT1A”. The term as used herein encompasses full-length and/or unprocessed SETDB1 as well as any intermediate resulting from processing in the cell. SETDB1 can exists as a soluble protein; thus, the term as used herein may refer to the full length protein. The term also encompasses naturally occurring variants of SETDB1 (e.g., splice variants or allelic variants). The protein may additionally contain a tag, such as a His-tag or Fc-tag. The amino acid sequence of exemplary human full-length SETDB1 protein can e.g. be found under UniProtKB Accession No. Q15047.

The terms “subject”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the disclosure include, but are not restricted to, any member of the subphylum Chordata including primates (e.g., humans, monkeys and apes, and includes species of monkeys such from the genus Macaca (e.g., cynomolgous monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of a therapy and/or the analytical/determination methods of the present disclosure. However, it will be understood that the aforementioned terms do not imply that symptoms are present.

As used herein, the terms “stratifying” and “classifying” are used interchangeably herein to refer to sorting of subjects into different strata or classes based on the features of a particular physiological or pathophysiological state or condition. For example, stratifying a population of subjects according to whether they are likely to respond to a therapy (e.g., immunotherapy) involves assigning the subjects based on levels of response to therapy biomarkers including SETDB1, in cancer cells.

As used herein, the terms “treatment”, “treating” and the like refer to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with a T-cell dysfunctional disorder are mitigated or eliminated, including, but are not limited to, reducing the proliferation of (or destroying) cancerous cells, reducing pathogen infection, decreasing symptoms resulting from the disease, increasing the quality of life of those suffering from the disease, decreasing the dose of other medications required to treat the disease, and/or prolonging survival of individuals.

As used herein, the term “tumour” refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. The term “non-metastatic” refers to a cancer that is benign or that remains at the primary site and has not penetrated into the lymphatic or blood vessel system or to tissues other than the primary site. Generally, a non-metastatic cancer is any cancer that is a Stage 0, I or II cancer. By “early stage cancer” is meant a cancer that is not invasive or metastatic or is classified as a stage 0, I or II cancer. The term “late stage cancer” generally refers to a Stage III or IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer (kidney cancer), carcinoma, retinoblastoma, melanoma, brain cancer, non-small cell lung cancer, squamous cell cancer of the head and neck, endometrial cancer, multiple myeloma, mesothelioma, rectal cancer and esophageal cancer. In an exemplary embodiment, the cancer is breast cancer or melanoma.

Each embodiment described herein is to be applied mutatis mutandis to each and every embodiment unless specifically stated otherwise.

### 2. Proteinaceous Molecules

The present invention is based, in part, on the determination that proteinaceous molecules corresponding to a nuclear localisation sequence (NLS) site of a SETDB1 polypeptide are useful for reducing the nuclear localization of the SETDB1 polypeptide. The present inventors identified that the mechanism by which nuclear shuttling of SETDB1 occurs was through complexing with importin-α (IMPα). Thus, the inventors have conceived that inhibition of the binding between a SETDB1 polypeptide and an IMPα polypeptide may be used for the treatment or prevention of cancer.

### 2.1 SETDB1 Proteinaceous Molecules

In one aspect, the present invention provides proteinaceous molecules that correspond to a NLS of a SETDB1 polypeptide. An example of such includes a polypeptide that comprises an amino acid sequence corresponding to residues 206 to 232 of the wild-type human SETDB1 polypeptide.

The amino acid sequence of human SETDB1 (Uniprot Accession No. Q15047) is presented in SEQ ID NO: 1, shown below with the NLS identified in bold and underlined typeface.

[SEQ ID NO: 1]
MSSLPGCIGLDAATATVESEEIAELQQAVVEELGISMEELRHFIDEELEKMDCVQQRKKQLA
ELETWVIQKESEVAHVDQLFDDASRAVTNCESLVKDFYSKLGLQYRDSSSEDESSRPTEIIEI
PDEDDDVLSIDSGDAGSRTPKDQKLREAMAALRKSAQDVQKFMDAVNKKSSSQDLHKGTL
SQMSGELSKDGDLIVSMRILGKKRTKTWHKGTLIAIQTVGPGKKYKVKFDNKGKSLLSGNH
IAYDYHPPADKLYVGSRVVAKYKDGNQVWLYAGIVAETPNVKNKLRFLIFFDDGYASYVTQS
ELYPICRPLKKTWEDIEDISCRDFIEEYVTAYPNRPMVLLKSGQLIKTEWEGTWWKSRVEEV
DGSLVRILFLDDKRCEWIYRGSTRLEPMFSMKTSSASALEKKQGQLRTRPNMGAVRSKGP
VVQYTQDLTGTGTQFKPVEPPQPTAPPAPPFPPAPPLSPQAGDSDLESQLAQSRKQVAKK
STSFRPGSVGSGHSSPTSPALSENVSGGKPGINQTYRSPLGSTASAPAPSALPAPPAPPVF
HGMLERAPAEPSYRAPMEKLFYLPHVCSYTCLSRVRPMRNEQYRGKNPLLVPLLYDFRRM
TARRRVNRKMGFHVIYKTPCGLCLRTMQEIERYLFETGCDFLFLEMFCLDPYVLVDRKFQP
YKPFYYILDITYGKEDVPLSCVNEIDTTPPPQVAYSKERIPGKGVFINTGPEFLVGCDCKDGC
RDKSKCACHQLTIQATACTPGGQINPNSGYQYKRLEECLPTGVYECNKRCKCDPNMCTNR
LVQHGLQVRLQLFKTQNKGWGIRCLDDIAKGSFVCIYAGKILTDDFADKEGLEMGDEYFANL
DHIESVENFKEGYESDAPCSSDSSGVDLKDQEDGNSGTEDPEESNDDSSDDNFCKDEDFS
TSSVWRSYATRRQTRGQKENGLSETTSKDSHPPDLGPPHIPVPPSIPVGGCNPPSSEETPK
NKVASWLSCNSVSEGGFADSDSHSSFKTNEGGEGRAGGSRMEAEKASTSGLGIKDEGDIK
QAKKEDTDDRNKMSVVTESSRNYGYNPSPVKPEGLRRPPSKTSMHQSRRLMASAQSNPD
DVLTLSSSTESEGESGTSRKPTAGQTSATAVDSDDIQTISSGSEGDDFEDKKNMTGPMKRQ
VAVKSTRGFALKSTHGIAIKSTNMASVDKGESAPVRKNTRQFYDGEESCYIIDAKLEGNLGR
YLNHSCSPNLFVQNVFVDTHDLRFPWVAFFASKRIRAGTELTWDYNYEVGSVEGKELLCCC
GAIECRGRLL.

Accordingly, in one aspect of the invention, there is provided an isolated or purified proteinaceous molecule represented by Formula (I).

Formula (I)
Z₁GKKRTKTWHKGTLIAIQTVGX₁GKKYKVKZ₂

wherein:
   Z₁ and Z₂ are independently absent or are independently selected from at least one of a proteinaceous moiety comprising from about 1 to about 50 amino acid residues (and all inteter resides in between), and a protecting moiety; and
   X₁ is selected from nonpolar/neutral amino acid residues including A, G, I, L, M, F, P, W, V, and Nle.

In some embodiments, Z₁ is absent. In other embodiments, Z₁ consists of 10, 9, 8, 7, 6, 5, 4, 3, 2 or 1 amino acid residues. In some embodiments the amino acid residues in Z₁ are independently selected from any amino acid residue.

In some embodiments, Z₂ is absent. In other embodiments, Z₂ consists of 10, 9, 8, 7, 6, 5, 4, 3, 2 or 1 amino acid residues. In some embodiments the amino acid residues in Z₂ are independently selected from any amino acid residue.

In some embodiments, X₁ is selected from nonpolar/neutral amino acid residues including Ala, Gly, Ile, Leu, Met, Phe, Pre, Trp, Val, and Nle. In particular embodiments, X₁ is selected from Pro and Leu, most especially Pro.

In some embodiments, the isolated or purified proteinaceous molecule of Formula I comprises, consists, or consists essentially of an amino acid sequence represented by any one of SEQ ID NO: 2 or 3:

SEQ ID NO: 2
GKKRTKTWHKGTLIAIQTVGPGKKYKVK
SEQ ID NO: 3
GKKRTKTWHKGTLIAIQTVGLGKKYKVK

In some embodiments, the proteinaceous molecule of Formula I has at least about 80%, 81%, 82%, 83%, 84%, 85%, 86%, 87%, 88%, 89%, 90%, 91%, 92%, 93%, 94%, 95%, 96%, 97%, 98%, or 99% sequence similarity to the amino acid sequence of SEQ ID NO: 2 or 3. In some embodiments, the proteinaceous molecule of Formula I has at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 2 or 3.

In some embodiments where the proteinaceous molecules of the invention comprise an N- and/or C-terminus, the proteinaceous molecules of the invention have a primary, secondary or tertiary amide, a hydrazide, a hydroxamide or a free-carboxyl group at the C-terminus and/or a primary amine or acetamide at the N-terminus. In some embodiments, the proteinaceous molecules of the invention are cyclic peptides and, thus, may not comprise N- and/or C-terminal amino acid residues.

The present invention also contemplates proteinaceous molecules that are variants of SEQ ID NO: 1 and 3. Such “variant” proteinaceous molecules include proteinaceous molecules derived from SEQ ID NO: 2 or SEQ ID NO: 3 by deletion or addition of one or more amino acids to the N-terminal and/or C-terminal end of the proteinaceous molecule, deletion or addition of one or more amino acids at one or more sites in the proteinaceous molecule, or substitution of one or more amino acids at one or more sites in the proteinaceous molecule.

Variant proteinaceous molecules encompassed by the present invention are biologically active, that is, they continue to possess the desired biological activity of the proteinaceous molecules. Such variants may result from, for example, genetic polymorphism or from human manipulation.

The proteinaceous molecules of SEQ ID NO: 1 or 2 may be altered in various ways, including amino acid substitutions, deletions, truncations and insertions. Methods for such manipulations are generally known in the art. For example, amino acid sequence variants of SEQ ID NO: 2 or 3 may be prepared by mutagenesis of nucleic acids encoding the amino acid sequence of any one of SEQ ID NO: 2 or 3. Methods for mutagenesis and nucleotide sequence alterations are well known in the art (for example, Kunkel, 1985; Kunkel et al., 1987; U.S. Pat. No. 4,873,192; and Watson et al., 1987). Guidance as to appropriate amino acid substitutions that do not affect biological activity of the proteinaceous molecules of interest may be found in the model of Dayhoff et al., 1978. Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of the proteinaceous molecules of SEQ ID NO: 2 or 3. Recursive ensemble mutagenesis (REM), a technique which enhances the frequency of functional mutants in the libraries, can be used in combination with screening assays to identify active variants (Arkin and Yourvan, 1992; Delgrave et al., 1993). Conservative substitutions, such as exchanging one amino acid with another having similar properties, may be desirable as discussed in more detail below.

Variant proteinaceous molecules of the invention may contain conservative amino acid substitutions at various locations along their sequence, as compared to a parent (e.g., reference) amino acid sequence, such as SEQ ID NO: 2 or 3. A “conservative amino acid substitution” is one in which the amino acid residue is replaced with an amino acid residue having a similar side chain. Families of amino acid residues having similar side chains have been defined in the art as discussed in detail below.

Acidic: The residue has a negative charge due to loss of a proton at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with protons at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residue is charged at physiological pH and, therefore, includes amino acids having acidic or basic side chains, such as glutamic acid, aspartic acid, arginine, lysine and histidine.

Hydrophobic: The residue is not charged at physiological pH and the residue is repelled by aqueous solution so as to seek the inner positions in the conformation of a peptide in which it is contained when the peptide is in aqueous medium at physiological pH. Amino acids having a hydrophobic side chain include tyrosine, valine, isoleucine, leucine, methionine, norleucine, phenylalanine and tryptophan.

Neutral/polar: The residues are not charged at physiological pH but the residue is not sufficiently repelled by aqueous solutions so that it would seek inner positions in the conformation of a peptide in which it is contained when the peptide is in aqueous medium at physiological pH. Amino acids having a neutral/polar side chain include asparagine, glutamine, cysteine, histidine, serine and threonine.

This description also characterizes certain amino acids as “small″” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., 1979; and Gonnet et al., 1992), however, include proline in the same group as glycine, serine, alanine and threonine. Accordingly, for the purposes of the present invention, proline is classified as a “small” amino acid.

The degree of attraction or repulsion required for classification as polar or non-polar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small amino acid residues are, of course, always non-aromatic. Dependent on their structural properties, amino acid residues may fall in two or more classes. For the naturally-occurring protein amino acids, sub-classification according to this scheme is presented in Table 1.

TABLE 1
Amino Acid Sub-Classification

| SUB-CLASSES | AMINO ACIDS |
|---|---|
| Acidic | Aspartic acid, Glutamic acid |
| Basic | Noncyclic: Arginine, lysine; |
| | Cyclic: histidine |
| Charged | Aspartic acid, glutamic acid, arginine, lysine, |
| | histidine |
| Small | Glycine, serine, alanine, threonine, proline |
| Nonpolar/neutral | Alanine, glycine, isoleucine, leucine, methionine, |
| | phenylalanine, proline, tryptophan, valine |
| Polar/neutral | Asparagine, histidine, glutamine, cysteine, serine, |
| | threonine, tyrosine |
| Polar/negative | Aspartic acid, glutamic acid |
| Polar/positive | Lysine, arginine |
| Polar/large | Asparagine, glutamine |
| Polar | Arginine, asparagine, aspartic acid, cysteine, |
| | glutamic acid, glutamine, histidine, lysine, serine, |
| | threonine, tyrosine |
| Hydrophobic | Tyrosine, valine, isoleucine, leucine, methionine, |
| | phenylalanine, tryptophan |
| Aromatic | Tryptophan, tyrosine, phenylalanine |
| Residues that influence | Glycine and proline |
| chain orientation | |

Conservative amino acid substitution also includes groupings based on side chains. For example, a group of amino acids having aliphatic side chains is glycine, alanine, valine, leucine, isoleucine and norleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartic acid with a glutamic acid, a threonine with a serine, or a similar replacement of an amino acid with a structurally related amino acid will not have a major effect on the properties of the resulting variant peptide of the invention. Whether an amino acid change results in a proteinaceous molecule that inhibits or reduces the nuclear localization of SETDB1, can readily be determined by assaying its activity. Conservative substitutions are shown in Table 2 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
EXEMPLARY AND PREFERRED
AMINO ACID SUBSTITUTIONS

| ORIGINAL | | PREFERRED |
|---|---|---|
| RESIDUE | EXEMPLARY SUBSTITUTIONS | SUBSTITUTIONS |
| Ala | Val, Leu, Ile | Val |
| Mlknb | Lys, Gln, Asn | Lys |
| Arg | | |
| Asn | Gln, His, Lys, Arg | Gln |
| Asp | Glu | Glu |
| Cys | Ser | See |
| Gln | Asn, His, Lys | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Vel, Ile, Ala | Leu |
| Pro | Gly | Gly |
| Ser | Thr | Thr |
| Thr | Ser | Ser |
| Trp | Tyr | Tyr |

Alternatively, similar amino acids for making conservative substitutions can be grouped into three categories based on the identity of the side chains. The first group includes glutamic acid, aspartic acid, arginine, lysine and histidine, which all have charged side chains; the second group includes glycine, serine, threonine, cysteine, tyrosine, glutamine and asparagine; and the third group includes leucine, isoleucine, valine, alanine, proline, phenylalanine, tryptophan, methionine and norleucine, as described in Zubay, Biochemistry, third edition, Wm.C. Brown Publishers (1993).

Thus n-essential amino acid residue in a proteinaceous molecule of the invention is typically replaced with another amino acid residue from the same side chain family. Alternatively, mutations can be introduced randomly along all or part of the coding sequence of a proteinaceous molecule of the invention, such as by saturation mutagenesis, and the resultant mutants can be screened for an activity of the parent polypeptide, as described for example herein, to identify mutants which retain that activity. Following mutagenesis of the coding sequences, the encoded proteinaceous molecule can be expressed recombinantly and its activity determined. A “non-essential” amino acid residue is a residue that can be altered from the reference sequence of an embodiment proteinaceous molecule of the invention without abolishing or substantially altering one or more of its activities. Suitably, the alteration does not substantially alter one of these activities, for example, the activity is at least 20%, 40%, 60%, 70% or 80% of that of the wild-type. By contrast, an “essential” amino acid residue is a residue that, when altered from the wild-type sequence of an embodiment x proteinaceous molecule of the invention, results in abolition of an activity of the parent molecule such that less than 20% of the wild-type activity is present.

Accordingly, the present invention also contemplates variants of the proteinaceous molecule of SEQ ID NO: 2 or 3, wherein the variants are distinguished from the parent sequence by the addition, deletion, or substitution of one or more amino acid residues. In general, variants will display at least about 80%, 81%, 82%, 83%, 84%, 85%, 86%, 87%, 88%, 89%, 90%, 91%, 92%, 93%, 94%, 95%, 96%, 97%, 98%, 99% sequence similarity to a reference proteinaceous molecule sequence as, for example, set forth in SEQ ID NO: 2 or 3, as determined by sequence alignment programs described elsewhere herein using default parameters. Desirably, variants will have at least 80%, 81%, 82%, 83%, 84%, 85%, 86%, 87%, 88%, 89%, 90%, 91%, 92%, 93%, 94%, 95%, 96%, 97%, 98%, 99% sequence identity to a parent or reference proteinaceous molecule sequence as, for example, set forth in SEQ ID NO: 2 or 3, as determined by sequence alignment programs described herein using default parameters. Variants of SEQ ID NO: 2 or 3 which fall within the scope of a variant proteinaceous molecule of the invention, may differ from the parent molecule generally by at least 1, but by less than 5, 4, 3, 2 or 1 amino acid residue(s). In some embodiments, a proteinaceous molecule of the invention differs from the corresponding sequence in SEQ ID NO: 2 or 3 by at least 1, but by less than 5, 4, 3, 2 or 1 amino acid residue(s). In some embodiments, the amino acid sequence of the variant proteinaceous molecule of the invention comprises the proteinaceous molecule of Formula I. In particular embodiments, the variant proteinaceous molecule of the invention inhibits or reduces nuclear localization of SETDB1.

If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution.

In some embodiments, calculations of sequence similarity or sequence identity between sequences are performed as follows:

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 40%, more usually at least 50% or 60%, and even more usually at least 70%, 80%, 90% or 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percent identity between the two sequences is a function of the number of identical amino acid residues shared by the sequences at individual positions, taking into account the number of gaps and the length of each gap, which need to be introduced for optimal alignment of the two sequences. By contrast, the percent similarity between the two sequences is a function of the number of identical and similar amino acid residues shared by the sequences at individual positions, taking into account the number of gaps and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and Wunsch, (1970) algorithm which has been incorporated into the GAP program in the GCG software package (Devereaux, et al., 1984), using either a Blosum 62 matrix or a PAM250 matrix, and a gap weight of 16, 14, 12, 10, 8, 6, or 4 and a length weight of 1, 2, 3, 4, 5, or 6. In some embodiments, the percent identity or similarity between amino acid sequences can be determined using the algorithm of Meyers and Miller (1989, Cablos, 4: 11-17) which has been incorporated into the ALIGN program (version 2.0), using a PAM120 weight residue table, a gap length penalty of 12 and a gap penalty of 4.

The present invention also contemplates an isolated or purified proteinaceous molecule that is encoded by a polynucleotide sequence that hybridizes under stringency conditions as defined herein, especially under medium, high or very high stringency conditions, preferably under high or very high stringency conditions, to a polynucleotide sequence encoding the proteinaceous molecule of SEQ ID NO: 2 or 3 or the non-coding strand thereof. The invention also contemplates an isolated nucleic acid molecule comprising a polynucleotide sequence that hybridizes under stringency conditions as defined herein, especially under medium, high or very high stringency conditions, preferably under high or very high stringency conditions, to a polynucleotide sequence encoding the proteinaceous molecule of SEQ ID NO: 2 or 3, or the non-coding strand thereof.

As used herein, the term “hybridizes under stringency conditions” describes conditions for hybridization and washing and may encompass low stringency, medium stringency, high stringency and very high stringency conditions.

Guidance for performing hybridization reactions can be found in Ausubel, et al. (1998) Current Protocols in Molecular Biology (John Wiley and Sons, Inc.), in particular sections 6.3.1-6.3.6. Both aqueous and non-aqueous methods can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% bovine serum albumin (BSA), 1 mM EDTA, 0.5 M NaHPO₄ (pH 7.2), 7% sodium dodecyl sulfate (SDS) for hybridization at 65° C., and (i) 2×sodium chloride/sodium citrate (SSC), 0.1% SDS; or (II) 0.5% BSA, 1 mM EDTA, 40 mM NaHPO₄ (pH 7.2), 5% SDS for washing at room temperature. One embodiment of low stringency conditions includes hybridization in 6×SSC at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% bovine serum albumin (BSA), 1 mM EDTA, 0.5 M NaHPO₄ (pH 7.2), 7% SDS for hybridization at 65° C., and (i) 2×SSC, 0.1% SDS; or (II) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (II) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In some aspects of the present invention, there is provided an isolated or purified proteinaceous molecule of the invention that is encoded by a polynucleotide sequence that hybridizes under high stringency conditions to a polynucleotide sequence encoding the proteinaceous molecule of SEQ ID NO: 2 or 3, or the non-coding strand thereof. In certain embodiments, the isolated or purified proteinaceous molecule of the invention is encoded by a polynucleotide sequence that hybridizes under very high stringency conditions to a polynucleotide sequence encoding the proteinaceous molecule of SEQ ID NO: 2 or 3, or the non-coding strand thereof. One embodiment of very high stringency conditions includes hybridizing 0.5 M sodium phosphate, 7% SDS at 65° C., followed by one or more washes at 0.2×SSC, 1% SDS at 65° C. In some embodiments, the amino acid sequence of the variant proteinaceous molecule of the invention comprises the amino acid sequence of Formula I. In particular embodiments, the proteinaceous molecule of the invention inhibits or reduces nuclear localization SETDB1.

Other stringency conditions are well known in the art and a person skilled in the art will recognize that various factors can be manipulated to optimize the specificity of the hybridization. Optimization of the stringency of the final washes can serve to ensure a high degree of hybridization. For detailed examples, see Ausubel, et al. (1998) Current Protocols in Molecular Biology (John Wiley and Sons, Inc.), in particular pages 2.10.1 to 2.10.16 and Sambrook, et al. (1989) Molecular Cloning: A Laboratory Manual (Cold Spring Harbour Press), in particular Sections 1.101 to 1.104.

While stringent washes are typically carried out at temperatures from about 42° C. to 68° C., a person skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel, et al. (1998) Current Protocols in Molecular Biology (John Wiley and Sons, Inc.) at page 2.10.8). In general, the Tₘ of a perfectly matched duplex of DNA may be predicted as an approximation by the formula:

$${T}_{m} = 81.5 + 16.6 ( {log}_{10} M ) + 0.41 ( \% G + C ) - 0.63 ( \% formamide ) - ( 600 / length )$$

wherein: M is the concentration of Na⁺, preferably in the range of 0.01 M to 0.4 M; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5×Denhardt's solution (0.1% ficoll, 0.1% polyvinylpyrrolidone and 0.1% BSA), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

The proteinaceous molecule of the present invention also encompass a proteinaceous molecule comprising amino acids with modified side chains, incorporation of unnatural amino acid residues and/or their derivatives during peptide synthesis and the use of cross-linkers and other methods which impose conformational constraints on the proteinaceous molecules of the invention. Examples of side chain modifications include modifications of amino groups, such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with sodium borohydride; reductive alkylation by reaction with an aldehyde followed by reduction with sodium borohydride; and trinitrobenzylation of amino groups with 2,4,6-tri nitrobenzene sulfonic acid (TNBS).

The carboxyl group may be modified by carbodiimide activation through O-acylisourea formation followed by subsequent derivatization, for example, to a corresponding amide.

The guanidine group of arginine residues may be modified by formation of heterocyclic condensation products with reagents such as 2,3-butanedione, phenylglyoxal and glyoxal.

Examples of incorporating unnatural amino acids and derivatives during peptide synthesis include, but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, N_{δ}-acetyl-L-ornithine, sarcosine, 2-thienyl alanine, N_{ε}-acetyl-L-lysine, N_{ε}-methyl-L-lysine, N_{ε}-dimethyl-L-lysine, N_{ε}-formyl-L-lysine and/or D-isomers of amino acids. A list of unnatural amino acids contemplated by the present invention is shown in Table 3.

TABLE 3
NON-CONVENTIONAL AMINO ACIDS

| | |
|---|---|
| α-aminobutyric acid | L-N-methylalanine |
| α-amino-α-methylbutyrate | L-N-methylarginine |
| Aminocyclopropane-carboxylate | L-N-methylasparagine |
| Aminoisobutyric acid | L-N-methylaspartic acid |
| Aminonorbornyl-carboxylate | L-N-methylcysteine |
| Cyclohexylalanine | L-N-methylglutamine |
| Ayclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-methylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-γ-aminobutyrate |
| D-α-methylalanine | α-methylcyclohexylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylhistidine | N-(2-aminoethyl)glycine |
| D-α-methylisoleucine | N-(3-aminopropyl)glycine |
| D-α-methylleucine | N-amino-α-methylbutyrate |
| D-α-methyllysine | α-napthylalanine |
| D-α-methylmethionine | N-benzylglycine |
| D-α-methylornithine | N-(2-carbamylediyl)glycine |
| D-α-methylpheylalanine | N-(caramylmethyl)glycine |
| D-α-methylproline | N-(2-carboxyethyl)glycine |
| D-α-methylserine | N-(carboxymethyl)glycine |
| D-α-methylthreonine | N-cyclobutylglycine |
| D-α-methyltryptophan | N-cycloheptylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-2,2-diphenylethyl | N-(N-(3,3-diphenylpropyl |
| carbamylmethyl)glycine | carbnamylmethyl)glycine |
| 1-carboxy-1-1(2,2-diphenyl-ethyl | L-selenocysteine |
| amino)cyclopropane | |
| D-selenocysteine | L-selenomethionine |
| L-telluromethionine | L-O-methyl homoserine |
| L-S-ethyl cysteine | L-ornithine |
| Nε-acetyl-L-lysine | Nε-methyl-L-lysine |
| Nε-dimethyl-L-lysine | Nε-formyl-L-lysine |
| D-norleucine | D-norvaline |
| Nδ-acetyl-L-orthinine | |

In some embodiments, the proteinaceous molecules of the invention comprise at least one unnatural amino acid.

### 2.2 Bicyclic Proteinaceous Molecules

In some embodiments, the proteinaceous molecule is a bicyclic molecule comprising a polypeptide that comprises at least three cysteine residues, separated by at least two loop sequences, and a molecular scaffold which forms covalent bonds with the cysteine residues of the polypeptide such that at least two polypeptide loops are formed on the molecular scaffold, wherein the polypeptide comprises an amino acid sequence of:

Forumula (II)
Z₁C₁GKKRX₁KX₂WHC₂X₃GTLC₃IX₄IQTVGX₅GKKX₆KVKZ₂

wherein:
   C₁, C₂, and C₃ represent first, second and third cysteine residues, respectively
   Z₁ and Z₂ are independently absent or are independently selected from at least one of a proteinaceous moiety comprising from about 1 to about 50 amino acid residues (and all integer residues in between), and a protecting moiety;
   X₁ is selected from Thr, Arg, and modified forms thereof;
   X₂ is selected from Thr, Leu, and modified forms thereof;
   X₃ is selected from Lys, Gly, and modified forms thereof;
   X₄ is selected from Ala, Pro, and modified forms thereof;
   X₅ is selected from Pro, Lys, and modified forms thereof; and
   X₆ is selected from Tyr, Lys, and modified forms thereof.

In some preferred embodiments, Z₁ and Z₂ are both absent.

In some of the same embodiments and some alternative embodiments, X₁ is selected from Thr.

In some of the same embodiments and some alternative embodiments, X₂ is selected from Thr.

In some of the same embodiments and some alternative embodiments, X₃ is selected from Lys.

In some of the same embodiments and some alternative embodiments, X₄ is selected from Ala.

In some of the same embodiments and some alternative embodiments, X₅ is selected from Pro.

In some of the same embodiments and some alternative embodiments, X₆ is selected from Tyr.

In some more specific embodiments, the proteinaceous molecule is a bicyclic molecule comprising a polypeptide that comprises at least three cysteine residues, separated by at least two loop sequences, and a molecular scaffold which forms covalent bonds with the cysteine residues of the polypeptide such that at least two polypeptide loops are formed on the molecular scaffold, wherein the polypeptide comprises an amino acid sequence of:

Formula (III)
Z₁X₁C₁GKKRTKTWHC₂KGTLIAIQTVGX₂GC₃KKYKVKZ₂

or a modified derivative, or pharmaceutically acceptable salt, thereof;
wherein:
   C₁, C₂, and C₃ represent first, second and third cysteine residues, respectively;
   Z₁ and Z₂ are independently absent or are independently selected from at least one of a proteinaceous moiety comprising from about 1 to about 50 amino acid residues (and all integers in between), and a protecting moiety;
   X₁ is absent or alanine; and
   X₂ is selected from nonpolar/neutral amino acid residues including A, G, I, L, M, F, P, W, V, and Nle.

In some embodiments, Z₁ is absent. In other embodiments, Z₁ consists of 10, 9, 8, 7, 6, 5, 4, 3, 2 or 1 amino acid residues. In some embodiments the amino acid residues in Z₁ are independently selected from any amino acid residue.

In some embodiments, Z₂ is absent. In other embodiments, Z₂ consists of 10, 9, 8, 7, 6, 5, 4, 3, 2 or 1 amino acid residues. In some embodiments the amino acid residues in Z₂ are independently selected from any amino acid residue.

In some embodiments, Z₂ is absent.

In some embodiments, X₂ is selected from nonpolar/neutral amino acid residues including Ala, Gly, Ile, Leu, Met, Phe, Pro, Trp, Val, and Nle. In particular embodiments, X₁ is selected from Pro and Leu, most especially Pro.

In some other embodiments of this type, the polypeptide comprises an amino acid sequence of selected from Formula (III) or (IV), as shown below:

Formula (IV)
Z₁X₁C₁GKKRTKTWHC₂KGTLC₃IAIQTVGX₁GKKYKVKZ₂
Formula (V)
Z₁X₁C₁GKKRTKTWHC₂KGTLIAIQC₃TVGX₁GKKYKVKZ₂

In some embodiments, the proteinaceous molecule of Formulae II, III, or IV comprises, consists, or consists essentially of, an amino acid sequence selected from Table 4.

TABLE 4
Bicyclic Proteinaceous Molecules

| Amino Acid Sequence | SEQ ID NO: |
|---|---|
| C₁GKKRTKTWHC₂KGTLIAIQTVGX₁GC₃KKYKVK | 4 |
| C₁GKKRTKTWHC₂KGTLC₃IAIQTVGX₁GKKYKVK | 5 |
| C₁GKKRTKTWHC₂KGTLIAIQC₃TVGX₁GKKYKVK | 6 |

In some embodiments, the proteinaceous molecules of Formulas (II)-(V) have at least about 80%, 81%, 82%, 83%, 84%, 85%, 86%, 87%, 88%, 89%, 90%, 91%, 92%, 93%, 94%, 95%, 96%, 97%, 98%, or 99% sequence similarity to the amino acid sequence of any one of SEQ ID NOs: 3-5. In some embodiments, the proteinaceous molecules of Formula II-IV have at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NOs: 4-6.

In some embodiments, the proteinaceous molecules of the present invention are alternative types of cyclic molecules. Without wishing to be bound by theory, cyclization of peptides is thought to decrease the susceptibility of the peptides to degradation. In particular embodiments, the SETDB1 bicyclic peptide mimetics are cyclized using N-to-C cyclization (head to tail cyclization), preferably through an amide bond. Such SETDB1 bicyclic peptide mimetics do not possess N- or C-terminal amino acid residues. In particular embodiments, the SETDB1 bicyclic peptide mimetics have an amide-cyclized peptide backbone. In other embodiments, the peptides are cyclized using sidechain to side-chain cyclization, preferably through a disulfide bond a diselenide bond, a seleno-sulfur bond, a thioether bond such as a lanthionine bond, a selenoether bond, a triazole bond, a lactam bond or a dimethylene bond; especially through a disulfide bond.

### Molecular Scaffolds

The SETDB1 bicyclic peptide mimetics of the invention comprise, consist essentially of, or consist of, the polypeptide covalently bound to a molecular scaffold. Molecular scaffolds are described in, for example, International PCT Patent Publication No. WO 2009/098450 and references cited therein, particularly WO 2004/077062 and WO 2006/078161. As noted in the foregoing documents, the molecular scaffold may be a small molecule, such as a small organic molecule.

In some embodiments, the molecular scaffold may be, or may be based on, natural monomers such as nucleosides, sugars, or steroids. For example, the molecular scaffold may comprise a short polymer of such entities, such as a dimer or trimer.

In one embodiment, the molecular scaffold is a compound of known toxicity, for example, low toxicity. Examples of suitable compounds include cholesterols, nucleotides, steroids, or existing drugs such as temazepam.

In some embodiments, the molecular scaffold may be a macromolecule. In some embodiments, the molecular scaffold is a macromolecule composed of amino acids, nucleotides, or carbohydrates.

In some embodiments, the molecular scaffold comprises reactive groups that are capable of reacting with functional group(s) of the polypeptide to form covalent bonds.

The molecular scaffold may comprise chemical groups, such as amines, thiols, alcohols, ketones, aldehydes, nitriles, carboxylic acids, esters, alkenes, alkynes, azides, anhydrides, succinimides, maleimides, alkyl halides and acyl halides.

In some embodiments, the scaffold is an aromatic molecular scaffold (i.e., a scaffold comprising a (hetero)aryl group). These aromatic rings can optionally contain one or more heteroatoms (e.g., one or more of N, O, S, and P), such as thienyl rings, pyridyl rings, and furanyl rings. The aromatic rings can be optionally substituted. The aryl rings can also be optionally substituted. Suitable substituents include alkyl groups (which can optionally be substituted), other aryl groups (which may themselves be substituted), heterocyclic rings (saturated or unsaturated), alkoxy groups (which is meant to include aryloxy groups (e.g., phenoxy groups)), hydroxy groups, aldehyde groups, nitro groups, amine groups (e.g., unsubstituted, or mono- or di-substituted with aryl or alkyl groups), carboxylic acid groups, carboxylic acid derivatives (e.g., carboxylic acid esters, amides, etc.), halogen atoms (e.g., Cl, Br, and I), and the like.

Suitably, the scaffold comprises a tris-substituted (hetero)aromatic or (hetero)alicyclic moiety, for example a tris-methylene substituted (hetero)aromatic or (hetero)alicyclic moiety. The (hetero)aromatic or (hetero)alicyclic moiety is suitably a six membered ring structure, preferably tris-substituted such that the scaffold has a 3-fold symmetry axis.

In some embodiments, the scaffold is a tris-methylene (hetero)aryl moiety, for example a 1,3,5-tris methylene benzene moiety. In these embodiments, the corresponding scaffold molecule suitably has a leaving group on the methylene carbons. The methylene group then forms the R1 moiety of the alkylamino linkage as defined herein. In these methylene-substituted (hetero)aromatic compounds, the electrons of the aromatic ring can stabilize the transition state during nucleophilic substitution. Thus, for example, benzyl halides are 100-1000 times more reactive towards nucleophilic substitution than alkyl halides that are not connected to a (hetero)aromatic group.

In embodiments of this type, the scaffold and scaffold molecule have the general formula:

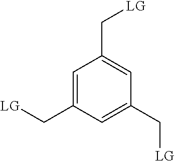

wherein LG represents a leaving group as described further below for the scaffold molecule, or LG (including the adjacent methylene group forming the R1 moiety of the alkylamino group) represents the alkylamino linkage to the peptide in the conjugates of the invention.

In some embodiments, the group LG above may be a halogen such as, but not limited to, a bromine atom, in which case the scaffold molecule is 1,3,5-Tris(bromomethyl)benzene (TBMB). Another suitable molecular scaffold molecule is 2,4,6-tris(bromomethyl) mesitylene. It is similar to 1,3,5-tris(bromomethyl) benzene but contains additionally three methyl groups attached to the benzene ring. In the case of this scaffold, the additional methyl groups may form further contacts with the peptide and hence add additional structural constraint. Thus, a different diversity range is achieved than with 1,3,5-Tris(bromomethyl)benzene.

Another preferred molecule for forming the scaffold for reaction with the peptide by nucleophilic substitution is 1,3,5-tris(bromoacetamido)benzene (TBAB):

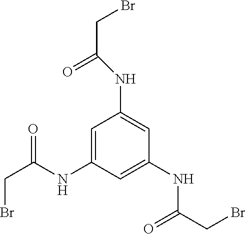 [SMILES: O=C(CBr)Nc1cc(NC(=O)CBr)cc(NC(=O)CBr)c1]

In some alternative embodiments, the scaffold is a non-aromatic molecular scaffold (e.g., a scaffold comprising a (hetero)alicyclic group). As used herein, “(hetero)alicyclic” refers to a homocyclic or heterocyclic saturated ring. The ring can be unsubstituted, or it can be substituted with one or more substituents. The substituents can be saturated or unsaturated, aromatic or nonaromatic, and examples of suitable substituents include those recited above in the discussion relating to substituents on alkyl and aryl groups. Furthermore, two or more ring substituents can combine to form another ring, so that “ring”, as used herein, is meant to include fused ring systems. In these embodiments, the alicyclic scaffold is preferably 1,1′,1″-(1,3,5-triazinane-1,3,5-triyl)triprop-2-en-1-one (TATA).

In some alternative embodiments the molecular scaffold may have a tetrahedral geometry such that reaction of four functional groups of the encoded peptide with the molecular scaffold generates not more than two product isomers. Other geometries are 5 also possible; indeed, an almost infinite number of scaffold geometries is possible, leading to greater possibilities for peptide ligand diversification.

The peptides used to form the bicyclic peptides of the invention comprise cysteines that are used to form thioether bonds to the scaffold, with replacement of the terminal —SH group of cysteine by —NH₂.

The bicyclic peptides of the present invention have a number of advantageous properties which enable them to be considered as beneficial drug-like molecules for injection, inhalation, nasal, ocular, oral or topical administration. Such advantageous properties include:
species cross-reactivity, which is a typical requirement for preclinical pharmacodynamics and pharmacokinetic evaluation;
protease stability, as bicyclic peptide ligands ideally demonstrate stability to plasma proteases, epithelial (“membrane-anchored”) proteases, gastric and intestinal proteases, lung surface proteases, intracellular proteases, and the like. Protease stability should be maintained between different species such that a bicycle peptide candidates can be developed in animal models as well as administered with confidence to humans;
desirable solubility profile, which is a function of the proportion of charged and hydrophilic versus hydrophobic residues and intra/inter-molecular H-bonding, which is important for formulation and absorption purposes; and
an optimal elimination half-life. Depending upon the clinical indication and treatment regimen, it may be required to develop a bicyclic peptide for short exposure in an acute illness management setting, or develop a bicyclic peptide with enhanced retention. It is therefore optimal for the management of more chronic disease states and cancers.

Other factors driving the desirable elimination half-life are requirements of sustained exposure for maximal therapeutic efficiency versus the accompanying toxicology due to sustained exposure of the agent.

In some embodiments, the molecular scaffold may comprise or may consist of tris(bromomethyl)benzene, especially 1,3,5-tris(bromomethyl)benzene (“TBMB”), or a derivative thereof. In some particularly preferred embodiments, the molecular scaffold is 1,3,5-(tribromomethyl)benzene).

In some other embodiments, the molecular scaffold is 2,4,6-tris(bromomethyl)mesitylene. This molecule is similar to 1,3,5-tris(bromomethyl)benzene but contains three additional methyl groups attached to the benzene ring. This has the advantage that the additional methyl groups may form further contacts with the polypeptide and hence add additional structural constraint.

Scaffold reactive groups that could be used on the molecular scaffold to react with thiol groups of cysteines are alkyl halides (or also names halogenoalkanes or haloalkanes).

Examples include bromomethylbenzene (the scaffold reactive group exemplified by TBMB) or iodoacetamide. Other scaffold reactive groups that are used to selectively couple compounds to cysteines in proteins are maleimides. Examples of maleimides which may be used as molecular scaffolds in the invention include: tris-(2-maleimidoethyl)amine, tris-(2-maleimidoethyl)benzene, tris-(maleimido)benzene. Selenocysteine is also a natural amino acid which has a similar reactivity to cysteine and can be used for the same reactions. Thus, wherever cysteine is mentioned, it is typically acceptable to substitute selenocysteine unless the context suggests otherwise.

### 2.3 Further N- or C-Termini Modifications.

In some embodiments described above, the N- and/or C-termini of the proteinaceous molecule may be subject to further modifications. For example, additional amino acids or other substituents may be added to the N- or C-termini, if present, of the proteinaceous molecule of the invention. In some embodiments, the proteinaceous molecules of the invention may form part of a longer sequence with additional amino acids added to either or both of the N- and C-termini.

For particular uses and methods of the invention, proteinaceous molecules with high levels of stability may be desired, for example, to increase the half-life of the proteinaceous molecule in a subject. Thus, in some embodiments, the proteinaceous molecules of the present invention comprise a stabilizing moiety or protecting moiety. The stabilizing moiety or protecting moiety may be coupled at any point on the peptide. Suitable stabilizing or protecting moieties include, but are not limited to, polyethylene glycol (PEG), a glycan or a capping moiety, including an acetyl group, pyroglutamate or an amino group. In preferred embodiments, the acetyl group and/or pyroglutamate are coupled to the N-terminal amino acid residue of the proteinaceous molecule. In particular embodiments, the N-terminus of the proteinaceous molecule is an acetamide. In preferred embodiments, the amino group is coupled to the C-terminal amino acid residue of the proteinaceous molecule. In particular embodiments, the proteinaceous molecule has a primary, secondary or tertiary amide, a hydrazide or a hydroxamide at the C-terminus; particularly a primary amide at the C-terminus. In preferred embodiments, the PEG is coupled to the N-terminal or C-terminal amino acid residue of the proteinaceous molecule or through the amino group of a lysine sidechain or other suitably modified side-chain, especially through the N-terminal amino acid residue such as through the amino group of the residue, or through the amino group of a lysine side-chain.

In preferred embodiments, the proteinaceous molecules of the present invention have a primary amide or a free carboxyl group (acid) at the C-terminus and a primary amine or acetamide at the N-terminus.

Although the proteinaceous molecules of the invention may inherently permeate membranes, membrane permeation may further be increased by the conjugation of a membrane permeating moiety to the proteinaceous molecule. Accordingly, in some embodiments, the proteinaceous molecules of the present invention comprise a membrane permeating moiety. The membrane permeating moiety may be coupled at any point on the proteinaceous molecule.

Suitable membrane permeating moieties include lipid moieties, cholesterol and proteins, such as cell penetrating peptides and polycationic peptides; especially lipid moieties.

Suitable cell penetrating peptides may include the peptides described in, for example, US 2009/0047272, US 2015/0266935 and US 2013/0136742. Accordingly, suitable cell penetrating peptides may include, but are not limited to, basic poly(Arg) and poly(Lys) peptides and basic poly(Arg) and poly(Lys) peptides containing non-natural analogues of Arg and Lys residues such as YGRKKRPQRRR (HIV TAT₄₇₋₅₇; SEQ ID NO: 7), RRWRRWWRRWWRRWRR (W/R; SEQ ID NO: 9), CWK₁₈ (AlkCWK₁₈; SEQ ID NO: 9), K₁₈WCCWK₁₈ (Di-CWK₁₈; SEQ ID NO: 10), WTLNSAGYLLGKINLKALAALAKKIL (Transportan; SEQ ID NO: 11), GLFEALEELWEAK (DipaLytic; SEQ ID NO: 12), K₁₆GGCRGDMFGCAK₁₆RGD (K16RGD; SEQ ID NO: 13), K₁₆GGCMFGCGG (PI; SEQ ID NO: 14), K₁₆ICRRARGDNPDDRCT (P2; SEQ ID NO: 15), KKWKMRRNQFWVKVQRbAK (B) bA (P3; SEQ ID NO: 16), VAYISRGGVSTYYSDTVKGRFTRQKYNKRA (P3a; SEQ ID NO: 17), IGRIDPANGKTKYAPKFQDKATRSNYYGNSPS (P9.3; SEQ ID NO: 18), KETWWETWWTEWSQPKKKRKV (Pep-1; SEQ ID NO: 19), PLAEIDGIELTY (Plae; SEQ ID NO: 20), K₁₆GGPLAEIDGIELGA (Kplae; SEQ ID NO: 21), K₁₆GGPLAEIDGIELCA (cKplae; SEQ ID NO: 22), WEAK(LAKA)₂-LAKH(LAKA)₂LKAC (HA2; SEQ ID NO: 23), (LARL)₆NHCH3 (LARL46; SEQ ID NO: 24), KLLKLLLKLWLLKLLL (Hel-11-7; SEQ ID NO: 25), (KKKK)₂GGC (KK; SEQ ID NO: 26), (KWKK)₂GCC (KWK; SEQ ID NO: 27), (RWRR)₂GGC (RWR; SEQ ID NO: 28), PKKKRKV (SV40 NLS7; SEQ ID NO: 29), PEVKKKRKPEYP (NLS12; SEQ ID NO: 30), TPPKKKRKVEDP (NLS12a; SEQ ID NO: 31), GGGGPKKKRKVGG (SV40 NLS13; SEQ ID NO: 32), GGGFSTSLRARKA (AV NLS13; SEQ ID NO: 33), CKKKKKKSEDEYPYVPN (AV RME NLS17; SEQ ID NO: 34), CKKKKKKKSEDEYPYVPN FSTSLRARKA (AV FP NLS28; SEQ ID NO: 35), LVRKKRKTEEESPLKDKDAKKSKQE (SV40 N1 NLS24; SEQ ID NO: 36), and K₉K2K₄K8GGK₅ (Loligomer; SEQ ID NO: 37); HSV-1 tegument protein VP22; HSV-1 tegument protein VP22r fused with nuclear export signal (NES); mutant B-subunit of Escherichia coli enterotoxin EtxB (H57S); detoxified exotoxin A (ETA); the protein transduction domain of the HIV-1 Tat protein, GRKKRRQRRRPPQ (SEQ ID NO: 38); the Drosophila melanogaster Antennapedia domain Antp (amino acids 43-58), RQIKIWFQNRRMKWKK (SEQ ID NO: 39); Buforin II, TRSSRAGLQFPVGRVHRLLRK (SEQ ID NO: 40); hClock (amino acids 35-47) (human Clock protein DNA-binding peptide), KRVSRNKSEKKRR (SEQ ID NO: 41); MAP (model amphipathic peptide), KIALKIALKALKAALKIA (SEQ ID NO: 42); K-FGF, AAVALLPAVLIALIAP (SEQ ID NO: 43); Ku70-derived peptide, comprising a peptide selected from the group comprising VPMLKE (SEQ ID NO: 44), VPMLK (SEQ ID NO: 45), PMLKE (SEQ ID NO: 46), or PMLK (SEQ ID NO: 47); Prion, Mouse Prpe (amino acids 1-28), MANLGYWLIALFVTMWTDVGLCKKRPKP (SEQ ID NO: 48); pVEC, LLIILRRRIRKQAHAHSK (SEQ ID NO: 49); Pep-I, KETWWETWWTEWSQPKKKRKV (SEQ ID NO: 66); SynBI, RGGRLSYSRRRFSTSTGR (SEQ ID NO: 50); Transportan, GWTLN SAGYLLGKINLKAIAAIAKKIL (SEQ ID NO: 51); Transportan-10, AGYLLGKINLKALAALAKKIL (SEQ ID NO: 52); CADY, Ac-GLWRALWRLLRSLWRLLWRA-cysteamide (SEQ ID NO: 53); Pep-7, SDLWEMMMVSIACQY (SEQ ID NO: 54); HN-1, TSPLNIHNGQKL (SEQ ID NO: 55); VT5, DPKGDPKGVTVTVTVTVTGKGDPKPD (SEQ ID NO: 56); plSL, RVIRVWFQNKRCKDKK (SEQ ID NO: 57), or GALFLGFLGGAAGSTMGAWSQPKSKRKV (MGP; SEQ ID NO: 58).

In preferred embodiments, the membrane permeating moiety is a lipid moiety, such as a C₁₀-C₂₀ fatty acyl group, especially stearoyl (octadecanoyl; C₁₈), palmitoyl (hexadecanoyl; C₁₆) or myristoyl (tetradecanoyl; C₁₄); most especially myristoyl. In preferred embodiments, the membrane permeating moiety is coupled to the N- or C-terminal amino acid residue or through the amino group of a lysine sidechain of the SETDB1 bicyclic peptide mimetic or other suitably modified side-chain, especially the N-terminal amino acid residue of the SETDB1 bicyclic peptide mimetic or through the amino group of a lysine side-chain. In particular embodiments, the membrane permeating moiety is coupled through the amino group of the N-terminal amino acid residue.

Accordingly, in another aspect of the present invention, there is provided an isolated or purified proteinaceous molecules represented by Formula VI:

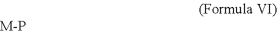

wherein:
M is a membrane permeating moiety; and
P is an isolated or purified proteinaceous molecule represented by any one of Formulas (I)-(V).

In some embodiments, M is coupled at any point on the proteinaceous molecule; especially to the N- or C-terminal amino acid residue or through the amino group of a lysine side-chain of the proteinaceous molecule or other suitably modified side-chain, more especially the N-terminal amino acid residue of the proteinaceous molecule or through the amino group of a lysine side-chain; most especially through the amino group of the N-terminal amino acid residue.

Suitable membrane permeating moieties and embodiments of the proteinaceous molecule represented by any one of Formulas (I)-(V) are as described herein.

### 2.4 Salts and Prodrugs

The proteinaceous molecules of the present invention may be in the form of salts or prodrugs. The salts of the proteinaceous molecules of the present invention are preferably pharmaceutically acceptable, but it will be appreciated that non-pharmaceutically acceptable salts also fall within the scope of the present invention.

The proteinaceous molecules of the present invention may be in crystalline form and/or in the form of solvates, for example, hydrates. Solvation may be performed using methods known in the art.

### 2.5 Synthesis of Linear Peptides

The peptides of the present invention may be prepared using recombinant DNA techniques or by chemical synthesis.

In some embodiments, the proteinaceous molecules of the present invention are prepared using recombinant DNA techniques. For example, the proteinaceous molecules of the invention may be prepared by a procedure including the steps of: (a) preparing a construct comprising a polynucleotide sequence that encodes the proteinaceous molecule of the invention and that is operably linked to a regulatory element; (b) introducing the construct into a host cell; (c) culturing the host cell to express the polynucleotide sequence to thereby produce the encoded proteinaceous molecule of the invention; and (d) isolating the proteinaceous molecule of the invention from the host cell. The proteinaceous molecules of the present invention may be prepared recombinantly using standard protocols, for example, as described in Klint et al. (2013); Sambrook et al. (1989); Ausubel et al. (1998); Coligan et al. (1997); and U.S. Pat. No. 5,976,567, the entire contents of which are hereby incorporated by reference.

Thus, the present invention also contemplates nucleic acid molecules which encode a proteinaceous molecule of the invention. Thus, in a further aspect of the present invention, there is provided an isolated nucleic acid molecule comprising a polynucleotide sequence that encodes the proteinaceous molecule of the invention or is complementary to a polynucleotide sequence that encodes a proteinaceous molecule of the invention, such as the proteinaceous molecules of any one of Formulas I-IV, of SEQ ID NOs: 1-5, or variant proteinaceous molecule as described herein.

The isolated nucleic acid molecules of the present invention may be DNA or RNA. When the nucleic acid molecule is in DNA form, it may be genomic DNA or cDNA. RNA forms of the nucleic acid molecules of the present invention are generally mRNA.

Although the nucleic acid molecules are typically isolated, in some embodiments, the nucleic acid molecules may be integrated into or ligated to or otherwise fused or associated with other genetic molecules, such as an expression vector. Generally, an expression vector includes transcriptional and translational regulatory nucleic acid operably linked to the polynucleotide sequence. Accordingly, in another aspect of the invention, there is provided an expression vector comprising a polynucleotide sequence that encodes a SETDB1 bicyclic peptide mimetic of the invention, such as the proteinaceous molecules of any one of Formulas I-IV, of SEQ ID NOs: 1-5, or variant proteinaceous molecule as described herein.

Typical vectors contain transcription and translation terminators, transcription and translation initiation sequences and promoters useful for regulation of the expression of the nucleic acid. The vectors optionally comprise generic expression cassettes containing at least one independent terminator sequence, sequences permitting replication of the cassette in eukaryotes, prokaryotes or both, (e.g., shuttle vectors) and selection markers for both prokaryotic and eukaryotic systems. Vectors may be suitable for replication and integration in prokaryotes, eukaryotes, or both. See, Giliman and Smith (1979); Roberts et al. (1987); Berger and Kimmel; Sambrook et al., supra; and Ausubel et al., supra, the entire contents of which are incorporated by reference.

Expression vectors containing regulatory elements from eukaryotic viruses such as retroviruses are typically used for expression of nucleic acid sequences in eukaryotic cells. SV40 vectors include pSVT7 and pMT2. Vectors derived from bovine papilloma virus include pBV-IMTHA, and vectors derived from Epstein Bar Virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumour virus promoter, Rous sarcoma virus promoter, polyhedrin promoter or other promoters shown effective for expression in eukaryotic cells.

While a variety of vectors may be used, it should be noted that viral expression vectors are useful for modifying eukaryotic cells because of the high efficiency with which the viral vectors transfect target cells and integrate into the target cell genome. Illustrative expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000); Vigna and Naldini (2000); Kay et al. (2001); Athanasopoulos et al. (2000); and Walther and Stein (2000), the entire contents of which are incorporated by reference.

The polypeptide or peptide-encoding portion of the expression vector may comprise a naturally-occurring sequence or a variant thereof, which has been engineered using recombinant techniques. In one example of a variant, the codon composition of a polynucleotide encoding a proteinaceous molecule of the invention is modified to permit enhanced expression of the proteinaceous molecule of the invention in a mammalian host using methods that take advantage of codon usage bias, or codon translational efficiency in specific mammalian cell or tissue types as set forth, for example, in International Publication Nos. WO 99/02694 and WO 00/42215. Briefly, these latter methods are based on the observation that translational efficiencies of different codons vary between different cells or tissues and that these differences can be exploited, together with codon composition of a gene, to regulate expression of a protein in a particular cell or tissue type. Thus, for the construction of codon-optimized polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5%, 10%, 15%, 20%, 25%, 30%, more preferably 35%, 40%, 50%, 60%, 70% or more of the existing codons of a parent polynucleotide.

The expression vector is compatible with the cell in which it is introduced such that the proteinaceous molecule of the invention is expressible by the cell. The expression vector is introduced into the cell by any suitable means which will be dependent on the particular choice of expression vector and cell employed. Such means of introduction are well-known to those skilled in the art. For example, introduction can be effected by use of contacting (e.g., in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g., LIPOFECTIN®, LIPOFECTAMINE™, and the like, supplied by Life Technologies, Gibco BRL, Gaithersburg, Md.).

In some embodiments, the proteinaceous molecules of the invention may be produced inside a cell by introduction of one or more expression constructs, such as an expression vector, that comprise a polynucleotide sequence that encodes a proteinaceous molecule of the invention.

The invention contemplates recombinantly producing the proteinaceous molecule of the invention inside a host cell, such as a mammalian cell (e.g., Chinese hamster ovary (CHO) cell, mouse myeloma (NSO) cell, baby hamster kidney (BHK) cell or human embryonic kidney (HEK293) cell), yeast cell (e.g., Pichia pastor/s cell, Saccharomyces cerevisiae cell, Schizosaccharomyces pombe cell, Hansenula polymorpha cell, Kluyveromyces lactis cell, Yarrowia lipolytica cell or Arxula adeninivorans cell), or bacterial cell (e.g., E. coli cell, Corynebacterium glutamicum or Pseudomonas fluorescens cell).

For therapeutic applications, the invention also contemplates producing the proteinaceous molecule of the invention in vivo inside a cell of a subject, for example a SETDB1 overexpressing cell, such as a vertebrate cell, particularly a mammalian or avian cell, especially a mammalian cell.

In some embodiments, the proteinaceous molecules of the present invention are prepared using standard peptide synthesis methods, such as solution synthesis or solid phase synthesis. The chemical synthesis of the proteinaceous molecules of the invention may be performed manually or using an automated synthesizer. For example, the linear peptides may be synthesized using solid phase peptide synthesis using either Boc or Fmoc chemistry, as described in Merrifield (1963); Schnolzer et al. (1992); and Cardoso et al. (2015); the entire contents of which are incorporated by reference. Following deprotection and cleavage from the solid support, the linear peptides are purified using suitable methods, such as preparative chromatography.

In other embodiments, the proteinaceous molecules of the invention may be cyclised. Cyclisation may be performed using several techniques, as described in, for example, Davies (2003), the entire contents of which are incorporated by reference. In particular embodiments, the linear peptide is synthesized using solid phase peptide synthesis involving Boc-chemistry, starting with a cysteine residue at the N-terminus and ending with a thioester at the C-terminus. Following deprotection and cleavage from the resin, the peptide is cyclised via a thiolactone intermediate, which subsequently rearranges to an amine-cyclised peptide.

### 2.6 Synthesis of Bicyclic Peptides

The bicyclic peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al. (supra).

Thus, the invention also relates to manufacture of polypeptides or conjugate selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities. To extend the peptide, it may simply be extended chemically at its N-terminus or C-terminus or within the loops using orthogonally protected lysines (and analogues) using standard solid phase or solution phase chemistry. Standard protein chemistry may be used to introduce an activatable N- or C-terminus. Alternatively, additions may be made by fragment condensation or native chemical ligation e.g., as described in (Dawson et al., Science 1994, 266:776-779), or by enzymes, for example using subtiligase as described in (Chang et al., Proc Natl Acad Sci USA 1994, 91(26): 12544-8; or in Hikari et al., Bioorganic & Medicinal Chemistry Letters, 2008, 18(22), 6000-6003).

Alternatively, the peptides may be extended or modified by further conjugation through disulphide bonds. This has the additional advantage of allowing the first and second

peptide to dissociate from each other once within the reducing environment of the cell. In this case, the molecular scaffold (e.g., TBMB) could be added during the chemical synthesis of the first peptide so as to react with the three cysteine groups; a further cysteine could then be appended to the N-terminus of the first peptide, so that this cysteine only reacted with a free cysteine of the second peptide.

Similar techniques apply equally to the synthesis/coupling of two bicyclic and bispecific macrocycles, potentially creating a tetraspecific molecule. Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

In some embodiments, the SETDB1 bicyclic peptide mimetics of the invention may be produced inside a cell by introduction of one or more expression constructs, such as an expression vector, that comprise a polynucleotide sequence that encodes a SEDB1 bicyclic peptide mimetic of the invention.

The invention contemplates recombinantly producing the SETDB1 bicyclic peptide mimetic of the invention inside a host cell, such as a mammalian cell (e.g., Chinese hamster ovary (CHO) cell, mouse myeloma (NSO) cell, baby hamster kidney (BHK) cell or human embryonic kidney (HEK293) cell), yeast cell (e.g., Pichia pastoris cell, Saccharomyces cerevisiae cell, Schizosaccharomyces pombe cell, Hansenula polymorpha cell, Kluyveromyces lactis cell, Yarrowia lipolytica cell or Arxula adeninivorans cell), or bacterial cell (e.g., Escherichia coli cell, Corynebacterium glutamicum or Pseudomonas fluorescens cell).

For therapeutic applications, the invention also contemplates producing the SETDB1 bicyclic peptide mimetics of the invention in vivo inside a cell of a subject, for example a SETDB1 overexpressing cell, such as a vertebrate cell, particularly a mammalian or avian cell, especially a mammalian cell.

In some embodiments, the SETDB1 bicyclic peptide mimetics of the present invention are prepared using standard peptide synthesis methods, such as solution synthesis or solid phase synthesis. The chemical synthesis of the SETDB1 bicyclic peptide mimetics of the invention may be performed manually or using an automated synthesizer. For example, the linear peptides may be synthesized using solid phase peptide synthesis using either Boc or Fmoc chemistry, as described in Merrifield (1963) J Am Chem Soc, 85(14): 2149-2154; Schnolzer, et al. (1992) Int J Pept Protein Res, 40: 180-193 and Cardoso, et al. (2015) Mol Pharmacol, 88(2): 291-303, the entire contents of which are incorporated by reference. Following deprotection and cleavage from the solid support, the linear peptides are purified using suitable methods, such as preparative chromatography.

### 3. Pharmaceutical Compositions

In accordance with the present invention, the proteinaceous molecules are useful in compositions and methods for the treatment or prevention of a condition involving the nuclear localization of SETDB1, for example a cancer.

Thus, in some embodiments, the proteinaceous molecule of the present invention may be in the form of a pharmaceutical composition, wherein the pharmaceutical composition comprises a proteinaceous molecule of the invention and a pharmaceutically acceptable carrier or diluent.

The proteinaceous molecules of the invention may be formulated into the pharmaceutical compositions as neutral or salt forms.

As will be appreciated by those skilled in the art, the choice of pharmaceutically acceptable carrier or diluent will be dependent on the route of administration and on the nature of the condition and the subject to be treated. The particular carrier or delivery system and route of administration may be readily determined by a person skilled in the art. The carrier or delivery system and route of administration should be carefully selected to ensure that the activity of the proteinaceous molecule is not depleted during preparation of the formulation and the proteinaceous molecule is able to reach the site of action intact. The pharmaceutical compositions of the invention may be administered through a variety of routes including, but not limited to, intravenous, oral, rectal, topical, intranasal, intraocular, transmucosal, intestinal, enteral, intramuscular, subcutaneous, intramedullary, intrathecal, intraventricular, intracerebral, intravaginal, intravesical, or intraperitoneal administration. In some preferred embodiments, the pharmaceutical compositions of the invention may be administered intravenously. In some other preferred embodiments the pharmaceutical compositions of the invention may be administered orally.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

A person skilled in the art will readily be able to determine appropriate formulations for the proteinaceous molecules of the invention using conventional approaches. Techniques for formulation and administration may be found in, for example, Remington (1980) Remington's Pharmaceutical Sciences, Mack Publishing Co., Easton, Pa., latest edition; and Niazi (2009) Handbook of Pharmaceutical Manufacturing Formulations, Informa Healthcare, New York, second edition, the entire contents of which are incorporated by reference.

Identification of preferred pH ranges and suitable excipients, such as antioxidants, is routine in the art, for example, as described in Katdare and Chaubel (2006) Excipient Development for Pharmaceutical, Biotechnology and Drug Delivery Systems (CRC Press). Buffer systems are routinely used to provide pH values of a desired range and may include, but are not limited to, carboxylic acid buffers, such as acetate, citrate, lactate, tartrate and succinate; glycine; histidine; phosphate; tris(hydroxymethyl)aminomethane (Tris); arginine; sodium hydroxide; glutamate; and carbonate buffers. Suitable antioxidants may include, but are not limited to, phenolic compounds such as butylated hydroxytoluene (BHT) and butylated hydroxyanisole; vitamin E; ascorbic acid; reducing agents such as methionine or sulfite; metal chelators such as ethylene diamine tetraacetic acid (EDTA); cysteine hydrochloride; sodium bisulfite; sodium meta bisulfite; sodium sulfite; ascorbyl palmitate; lecithin; propyl gallate; and alpha-tocopherol.

For injection, the proteinaceous molecules of the invention may be formulated in aqueous solutions, suitably in physiologically compatible buffers such as Hanks' solution, Ringer's solution or physiological saline buffer. For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the proteinaceous molecules of the present invention can be formulated readily using pharmaceutically acceptable carriers well known in the art into dosages suitable for oral administration, which is also contemplated for the practice of the present invention. Such carriers enable the bioactive agents of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline and pyrogen-free water.

Pharmaceutical formulations for parenteral administration include aqueous solutions of the proteinaceous molecules of the invention in water-soluble form. Additionally, suspensions of the proteinaceous molecules of the invention may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilizers or agents that increase the solubility of the compounds to allow for the preparation of highly concentrated solutions.

Sterile solutions may be prepared by combining the active compounds in the required amount in the appropriate solvent with other excipients as described above as required, followed by sterilization, such as filtration. Generally, dispersions are prepared by incorporating the various sterilized active compounds into a sterile vehicle which contains the basic dispersion medium and the required excipients as described above. Sterile dry powders may be prepared by vacuum- or freeze-drying a sterile solution comprising the active compounds and other required excipients as described above.

Pharmaceutical preparations for oral use can be obtained by combining the proteinaceous molecules of the invention with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arable, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The proteinaceous molecules of the invention may be incorporated into modified-release preparations and formulations, for example, polymeric microsphere formulations, and oil- or gel-based formulations.

In particular embodiments, the proteinaceous molecule of the invention may be administered in a local rather than systemic manner, such as by injection of the proteinaceous molecule directly into a tissue, which is preferably subcutaneous or omental tissue, often in a depot or sustained release formulation.

Furthermore, the proteinaceous molecule of the invention may be administered in a targeted drug delivery system, such as in a particle which is suitably targeted to and taken up selectively by a cell or tissue. In some embodiments, the proteinaceous molecule of the invention is contained in or otherwise associated with a vehicle selected from liposomes, micelles, dendrimers, biodegradable particles, artificial DNA nanostructure, lipid-based nanoparticles and carbon or gold nanoparticles. In illustrative examples of this type, the vehicle is selected from poly(lactic acid) (PLA), poly(glycolic acid) (PGA), poly(lactic-co-glycolic acid) (PLGA), polyethylene glycol) (PEG), PLA-PEG copolymers and combinations thereof.

In cases of local administration or selective uptake, the effective local concentration of the agent may not be related to plasma concentration.

It is advantageous to formulate the compositions in dosage unit form for ease of administration and uniformity of dosage. The determination of the novel dosage unit forms of the present invention is dictated by and directly dependent on the unique characteristics of the active material, the particular therapeutic effect to be achieved and the limitations inherent in the art of compounding active materials for the treatment of disease in living subjects having a diseased condition in which bodily health is impaired as herein disclosed in detail.

While the proteinaceous molecule of the invention may be the sole active ingredient administered to the subject, the administration of other cancer therapies concurrently with said proteinaceous molecule is within the scope of the invention. For example, the proteinaceous molecule of any one of Formulas I-IV, of any one of SEQ ID NOs: 2-6, or variant described herein may be administered concurrently with one or more cancer therapies, non-limiting examples of which include radiotherapy, surgery, chemotherapy, hormone ablation therapy, pro-apoptosis therapy and immunotherapy The proteinaceous molecule of the invention may be therapeutically used before treatment with the cancer therapy, may be therapeutically used after the cancer therapy or may be therapeutically used together with the cancer therapy.

Suitable radiotherapies include radiation and waves that induce DNA damage, for example, γ-irradiation, X-rays, UV irradiation, microwaves, electronic emissions and radioisotopes. Typically, therapy may be achieved by irradiating the localized tumour site with the above-described forms of radiations. It is most likely that all of these factors cause a broad range of damage to DNA, on the precursors of DNA, on the replication and repair of DNA and on the assembly and maintenance of chromosomes.

The dosage range for X-rays ranges from daily doses of 50-200 roentgens for prolonged periods of time such as 3-4 weeks, to single doses of 2000-6000 roentgens. Dosage ranges for radioisotopes vary widely and depend on the half life of the isotope, the strength and type of radiation emitted and the uptake by the neoplastic cells. Suitable radiotherapies may include, but are not limited to, conformal external beam radiotherapy (50-100 Gray given as fractions over 4-8 weeks), either single shot or fractionated high dose brachytherapy, permanent interstitial brachytherapy and systemic radioisotopes such as strontium 89. In some embodiments, the radiotherapy may be administered with a radiosensitizing agent. Suitable radiosensitizing agents may include, but are not limited to, efaproxiral, etanidazole, fluosol, misonidazole, nimorazole, temoporfin and tirapazamine.

Suitable chemotherapeutic agents may include, but are not limited to, antiproliferative/antineoplastic drugs and combinations thereof including alkylating agents (for example cisplatin, carboplatin, cyclophosphamide, nitrogen mustard, melphalan, chlorambucil, busulphan and nitrosoureas), antimetabolites (for example antifolates such as fluoropyridines like 5-fluorouracil and tegafur, raltitrexed, methotrexate, cytosine arabinoside and hydroxyurea), anti-tumour antibiotics (for example anthracydines like adriamycin, bleomycin, doxorubicin, daunomycin, epirubicin, idarubicin, mitomycin-C, dactinomycin and mithramycin), antimitotic agents (for example Vinca alkaloids like vincristine, vinblastine, vindesine and vinorelbine and taxoids like paditaxel and docetaxel), and topoisomerase inhibitors (for example epipodophyllotoxins like etoposide and teniposide, amsacrine, topotecan and camptothecin); cytostatic agents such as antiestrogens (for example tamoxifen, toremifene, raloxifene, droloxifene and idoxifene), estrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5a-reductase such as finasteride; agents which inhibit cancer cell invasion (for example metalloproteinase inhibitors like marimastat and inhibitors of urokinase plasminogen activator receptor function); inhibitors of growth factor function, for example such inhibitors include growth factor antibodies, growth factor receptor antibodies (for example the anti-erbb2 antibody trastuzumab (HERCEPTIN™) and the anti-EGFR antibody Cetuximab (C225)), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (Gefitinib, AZD1839), N-(3-ethynylphenyl)-6,7-bis(2-methoxyethoxy)quinazolin-4-amine (Erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family; anti-angiogenic agents such as those which inhibit the effects of vascular endothelial growth factor, (for example the anti-vascular endothelial cell growth factor antibody bevacizumab (AVASTIN™), compounds such as those disclosed in International Patent Publication Nos. WO 97/22596, WO 97/30035, WO 97/32856 and WO 98/13354) and compounds that work by other mechanisms (for example, linomide, inhibitors of integrin anb3 function and angiostatin); cyclin-dependent kinase inhibitors such as palbocidib, abemacidib, riboddib and alvoddib; vascular damaging agents such as Combretastatin A4 and compounds disclosed in International Patent Publication Nos. WO 99/02166, WO 00/40529, WO 00/41669, WO 01/92224, WO 02/04434 and WO 02/08213; antisense therapies, for example those which are directed to the targets listed above, such as ISIS 2503, an anti-ras antisense; and gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Suitable immunotherapy approaches may include, but are not limited to ex vivo and in vivo approaches to increase the immunogenicity of patient tumour cells such as transfection with cytokines including interleukin 2, interleukin 4 or granulocyte-colony stimulating factor; approaches to decrease T-cell anergy; approaches using transfected immune cells such as cytokine-transfected dendritic cells; approaches using cytokine-transfected tumour cell lines; and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

In some embodiments, the immune effector is a molecule targeting PD-L1, including, but not limited to, an anti-PD-L1 antibody, non-limiting examples of which include atezolizumab, avelumab, durvalumab, BMS-936559, BMS-935559, the antibodies described in International Patent Publication Nos. WO 2013/173223, WO 2013/079174, WO 2010/077634, WO 2011/066389, WO 2010/036959, WO 2007/005874, WO 2004/004771, WO 2006/133396, WO 2013/181634, WO 2012/145493 and Chinese Patent Publication No. CN101104640, clone EHi2, and clone 29E.2A3; CA-170; CA-327; BMS-202 (N-[2-[[[2-methoxy-6-[(2-methyl[1,1′-biphenyl]-3-yl)methoxy]-3-pyridinyl]methyl]amino]ethyl]-acetamide); BMS-8 (1-[[3-bromo-4-[(2-methyl[1,1′-biphenyl]-3-yl)methoxy]phenyl]methyl]-2-piperidinecarboxylic acid); the peptides described in Chang et al. (2015), especially (D) PPA-1; AUNP-12; and the peptides described in WO 2014/151634, the entire contents of which are incorporated by reference.

In some embodiments, the immune effector is a molecule targeting PD-1 including, but not limited to, an anti-PD-1 antibody, non-limiting examples of which include nivolumab, pembrolizumab, BGB-A317, the antibodies described in WO 2016/106159, WO 2009/114335, WO 2004/004771, WO 2013/173223, WO 2015/112900, WO 2008/156712, WO 2011/159877, WO 2010/036959, WO 2010/089411, WO 2006/133396, WO 2012/145493, WO 2002/078731, anti-mouse PD-1 antibody clone J43, anti-mouse antibody clone RMP1-14, ANB011 (TSR-042), AMP-514 (MED10680), WO 2006/121168, WO 2001/014557, WO 2011/110604, WO 2011/110621, WO 2004/072286 A1, WO 2004/056875, WO 2010/036959, WO 2010/029434, and WO 2013/02209; AMP-224; the compounds described in WO 2011/082400; the molecules and antibodies described in U.S. Pat. No. 6,808,710; the molecules and antibodies described in WO 2013/019906; the molecules described in WO 2003/011911; and the compounds described in WO 2013/132317, the entire contents of which are incorporated by reference.

In some embodiments, the immune effector is a molecule targeting PD-L2 including, but not limited to, an anti-PD-L2 antibody, non-limiting examples of which include the antibodies described in International Patent Publication No. WO 2010/036959, the entire content of which is incorporated by reference; and rHigM12B7.

In some embodiments, the immune effector is a molecule targeting CTLA-4 including, but not limited to, an anti-CTLA-4 antibody such as ipilimumab, tremelimumab, the antibodies described in WO 00/37504 A2, WO 01/14424 A2, US 2003/0086930 A1; and the compounds described in WO 2006/056464 A2, the entire contents of which are incorporated by reference.

Examples of other cancer therapies include phytotherapy, cryotherapy, toxin therapy or pro-apoptosis therapy. A person skilled in the art would appreciate that this list is not exhaustive of the types of treatment modalities available for cancer and other hyperplastic lesions.

It is well known that chemotherapy and radiation therapy target rapidly dividing cells and/or disrupt the cell cycle or cell division. These treatments are offered as part of treating several forms of cancer, aiming either at slowing their progression or reversing the symptoms of disease by means of a curative treatment. However, these cancer treatments may lead to an immunocompromised state and ensuing pathogenic infections and, thus, the present invention also extends to combination therapies, which employ a proteinaceous molecule of any one of Formulas I-IV, or any one of SEQ ID NOs: 1-5, or variants described herein, a cancer therapy and an anti-infective agent that is effective against an infection that develops or that has an increased risk of developing from an immunocompromised condition resulting from the cancer therapy. The anti-infective drug is suitably selected from antimicrobials, which may include, but are not limited to, compounds that kill or inhibit the growth of microorganisms such as viruses, bacteria, yeast, fungi, protozoa, etc. and thus include antibiotics, amebicides, antifungals, anti-protozoa Is, antimalarials, antituberculotics and antivirals. Anti-infective drugs also include within their scope anthelmintics and nematocides. Illustrative antibiotics include quinolones (e.g., amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, dinafloxacin, gatifloxacin, moxifloxacin; gemifloxacin; and garenoxacin), tetracyclines, glycylcyclines and oxazolidinones (e.g., chlortetracydine, demedocydine, doxycycline, lymecycline, methacycline, minocycline, oxytetracycline, tetracycline, tigecydine; linezolide, eperezolid), glycopeptides, aminoglycosides (e.g., amikacin, arbekadn, butirosin, dibekadn, fortimicins, gentamicin, kanamydn, menomydn, netilmicin, ribostamydn, sisomicin, spectinomydn, streptomycin, tobramycin), 3-lactams (e.g., imipenem, meropenem, biapenem, cefaclor, cefadroxil, cefamandole, cefatrizine, cefazedone, cefazolin, cefixime, cefmenoxime, cefodizime, cefonidd, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglydn, cephaloridine, cephalothin, cephapirin, cephradine, cefinetazole, cefoxitin, cefotetan, azthreonam, carumonam, flomoxef, moxalactam, amdinocilin, amoxicillin, ampicillin, azlocillin, carbenicillin, benzylpenicillin, carfecilin, doxacillin, didoxacillin, methidllin, mezlocillin, nacillin, oxacillin, penicillin G, piperacillin, sulbenicillin, temocillin, ticardllin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g., azithromycin, clarithromycin, erythromycin, oleandomycin, rokitamycin, rosaramicin, roxithromycin, troleandomycin), ketolides (e.g., telithromycin, cethromycin), coumermycins, lincosamides (e.g., clindamycin, lincomycin) and chloramphenicol.

Illustrative antivirals include abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine lamivudine/zidovudine, nelfinavir mesylate, nevirapine, oseltamivir phosphate, ribavirin, rimantadine hydrochloride, ritonavir, saquinavir, saquinavir mesylate, stavudine, valacydovir hydrochloride, zalcitabine, zanamivir and zidovudine.

Suitable amebicides or antiprotozoals include, but are not limited to, atovaquone, chloroquine hydrochloride, chloroquine phosphate, metronidazole, metronidazole hydrochloride and pentamidine isethionate. Anthelmintics can be at least one selected from mebendazole, pyrantel pamoate, albendazole, ivermectin and thiabendazole. Illustrative antifungals can be selected from amphotericin B, amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin and terbinafine hydrochloride. Suitable antimalarials include, but are not limited to, chloroquine hydrochloride, chloroquine phosphate, doxycycline, hydroxychloroquine sulfate, mefloquine hydrochloride, primaquine phosphate, pyrimethamine and pyrimethamine with sulfadoxine. Antituberculotics include but are not restricted to clofazimine, cycloserine, dapsone, ethambutol hydrochloride, isoniazid, pyrazinamide, rifabutin, rifampin, rifapentine, and streptomycin sulfate.

As previously described, the proteinaceous molecule may be compounded for convenient and effective administration in effective amounts with a suitable pharmaceutically acceptable carrier in dosage unit form. In some embodiments, a unit dosage form may comprise the active peptide of the invention in amount in the range of from about 0.25 pg to about 2000 mg. The active peptide of the invention may be present in an amount of from about 0.25 pg to about 2000 mg/mL of carrier. In embodiments where the pharmaceutical composition comprises one or more additional active ingredients, the dosages are determined by reference to the usual dose and manner of administration of the said ingredients.

### 4. Methods of Treatment

The present inventors have determined that blocking nuclear localisation of SETDB1 would be an effective treatment for diseases such as cancer (e.g., metastatic cancer). Accordingly, in some embodiments the present invention provides a method for treating cancer in a subject, the method comprising administering to the subject an agent that prevents or reduces nuclear localisation of SETDB1.

In some embodiments, the agent comprises a proteinaceous molecule described above or elsewhere herein. For example, the proteinaceous molecule may comprise an amino acid sequence corresponding to any one of Formulas (I)-(IV).

Without wishing to be bound by theory, the inventors have determined that SETDB1 is enriches in the nucleus of metastatic initiating tumour cells and dysfunctional terminally exhausted CD8+ T cells in subjects that are resistant to immunotherapy (e.g., immune checkpoint inhibitor therapy). Conversely, SETDB1 was found to be enriched in the cytoplasm and/or cell surface of subjects that responded to immunotherapy (e.g., immune checkpoint inhibitor therapy). Therefore, it is proposed that by preventing nuclear localisation of a SETDB1 polypeptide in a cell of the subject would prevent or reduce the likelihood of a subject being resistant to immunotherapy (e.g., immune checkpoint inhibitor therapy).

Accordingly, in another aspect of the invention, there is provided a method of inhibiting or reducing the nuclear localization of SETDB1, the method comprising contacting the cell with a proteinaceous molecule as described above or elsewhere herein.

In some embodiments, the proteinaceous molecule is an isolated or purified proteinaceous molecules represented by any one of Formulas (I)-(IV); particularly the proteinaceous molecules of any one of SEQ ID NOs: 2-6, or variant proteinaceous molecules described herein.

In another aspect of the invention, there is provided a use of the isolated or purified proteinaceous molecules of the invention, particularly the proteinaceous molecules of any one of Formulas I-IV, SEQ ID NOs: 2-6, or variant proteinaceous molecule described herein, for therapy or in the manufacture of a medicament for therapy. The invention also provides an isolated or purified proteinaceous molecules of the invention, particularly the proteinaceous molecule of Formulas (I)-(IV), SEQ ID NOs: 2-6, or variant proteinaceous molecule described herein, for use in therapy.

The present invention also provides a method of inhibiting or reducing nuclear localization of SETDB1 in a SETDB1-overexpressing cell, comprising contacting the cell with an inhibitor of the binding between a SETDB1 polypeptide and an importin-α polypeptide. In some embodiments the inhibitor comprises a proteinaceous molecule comprising, consisting or consisting essentially of an amino acid sequence corresponding to a NLS. The present invention also contemplates the use of a proteinaceous molecule comprising, consisting or consisting essentially of an amino acid sequence corresponding to a NLS for inhibiting or reducing nuclear localization of SETDB1 in a SETDB1-overexpressing cell; a proteinaceous molecule comprising, consisting or consisting essentially of an amino acid sequence corresponding to a NLS for use in inhibiting or reducing the nuclear localization of SETDB1 in a SETDB1-overexpressing cell; and in the manufacture of a medicament for such use.

In some embodiments, of any one of the above aspects, the SETDB1-overexpressing cell is a cancer stem cell or a non-cancer stem cell tumour cell, especially a cancer stem cell tumour cell.

Suitable embodiments of the proteinaceous molecule are as described herein.

Proteinaceous molecules comprising, consisting or consisting essentially of an amino acid sequence corresponding to an acetylation site as described herein, especially the proteinaceous molecules of any one of Formulas (I)-(IV), SEQ ID NOs: 2-6, or variant proteinaceous molecules, are useful for the inhibition of nuclear localization of SETDB1. Accordingly, the present inventors have conceived that the proteinaceous molecules are useful for treating or preventing a cancer in a subject. Thus, in another aspect, there is provided a method for treating or preventing a cancer in a subject wherein the cancer comprises at least one SETDB1-overexpressing cell, comprising administering to the subject a proteinaceous molecule comprising, consisting or consisting essentially of an amino acid sequence corresponding to an acetylation site. The present invention also extends to a use of a proteinaceous molecule comprising, consisting or consisting essentially of an amino acid sequence corresponding to an acetylation site for treating or preventing a cancer in a subject wherein the cancer comprises at least one SETDB1-overexpressing cell; and in the manufacture of a medicament for this purpose. A SETDB1 bicyclic peptide mimetic comprising, consisting or consisting essentially of an amino acid sequence corresponding to an acetylation site for use in treating or preventing a cancer in a subject wherein the cancer comprises at least one SETDB1-overexpressing cell is also contemplated.

The cancer may be any cancer involving overexpression of SETDB1. Suitable cancers may include, but are not limited to breast, prostate, lung, bladder, pancreatic, colon, liver, ovarian, kidney or brain cancer, or melanoma or retinoblastoma; especially breast cancer, lung cancer or melanoma; most especially breast cancer or melanoma; more especially breast cancer.

In some embodiments, the proteinaceous molecules comprising, consisting or consisting essentially of an amino acid sequence corresponding to an acetylation site as described herein are useful for treating, preventing and/or relieving the symptoms of a malignancy, particularly a metastatic cancer. In preferred embodiments, the SETDB1 bicyclic peptide mimetics are used for treating, preventing and/or relieving the symptoms of a metastatic cancer. Suitable types of metastatic cancer include, but are not limited to, metastatic breast, prostate, lung, bladder, pancreatic, colon, liver, ovarian, kidney or brain cancer, or melanoma or retinoblastoma. In some embodiments, the brain cancer is a glioma. In preferred embodiments, the metastatic cancer is metastatic breast cancer, lung cancer or melanoma; especially metastatic breast cancer or melanoma; most especially metastatic breast cancer.

The proteinaceous molecules are useful in methods involving SETDB1-overexpressing cells. In particular embodiments, the SETDB1-overexpressing cell is selected from a breast, prostate, testicular, lung, bladder, pancreatic, colon, melanoma, leukemia, retinoblastoma, liver, ovary, kidney or brain cell; especially a breast, lung or melanoma cell; most especially a breast or melanoma cell; more especially a breast cell. In preferred embodiments, the SETDB1-overexpressing cell is a breast epithelial cell, especially a breast ductal epithelial cell.

In some embodiments, the SETDB1-overexpressing cell is a cancer stem cell or a non-cancer stem cell tumour cell; especially a cancer stem cell tumour cell; most especially a breast cancer stem cell tumour cell. In some embodiments, the cancer stem cell tumour cell expresses CD24 and CD44, particularly CD44^{high}, CD24^{low}.

In some embodiments, the methods further comprise detecting overexpression of a SETDB1 gene in a tumour sample obtained from the subject, wherein the tumour sample comprises the cancer stem cell tumour cells and optionally the non-cancer stem cell tumour cells, prior to administering the proteinaceous molecule to the subject.

The proteinaceous molecules comprising, consisting or consisting essentially of an amino acid sequence corresponding to a lysine methylation site as described herein are suitable for treating an individual who has been diagnosed with a cancer, who is suspected of having a cancer, who is known to be susceptible and who is considered likely to develop a cancer, or who is considered to develop a recurrence of a previously treated cancer. The cancer may be hormone receptor negative. In some embodiments, the cancer is hormone receptor negative and is, thus, resistant to hormone or endocrine therapy. In some embodiments where the cancer is breast cancer, the breast cancer is hormone receptor negative. In some embodiments, the breast cancer is estrogen receptor negative and/or progesterone receptor negative.

There are numerous conditions involving SETDB1-overexpression in which the proteinaceous molecule comprising, consisting or consisting essentially of an amino acid sequence corresponding to a lysine methylation site as described herein may be useful. Accordingly, in another aspect of the invention, there is provided a method of treating or preventing a condition in a subject in respect of which inhibition or reduction of nuclear localization of SETDB1 is associated with effective treatment, comprising administering to the subject a proteinaceous molecule comprising, consisting or consisting essentially of an amino acid sequence corresponding to an acetylation site. The invention also provides a use of a proteinaceous molecule comprising, consisting or consisting essentially of an amino acid sequence corresponding to an acetylation site for treating or preventing a condition in a subject in respect of which inhibition or reduction of nuclear localization of SETDB1 is associated with effective treatment; a proteinaceous molecule comprising, consisting or consisting essentially of an amino acid sequence corresponding to an acetylation site for use in treating or preventing a condition in a subject in respect of which inhibition or reduction of nuclear localization of SETDB1 is associated with effective treatment; and a use of a proteinaceous molecule comprising, consisting or consisting essentially of an amino acid sequence corresponding to an acetylation site in the manufacture of a medicament for this purpose.

Non-limiting examples of conditions involving SETDB1-overexpression include cancer, infection, autoimmune disorders and respiratory disorders.

In some embodiments, the infection is a pathogenic infection. The infection may be selected from, but is not limited to, a viral, bacterial, yeast, fungal, helminth or protozoan infection. Viral infections contemplated by the present invention include, but are not restricted to, infections caused by HIV, hepatitis, influenza virus, Japanese encephalitis virus, Epstein-Barr virus, herpes simplex virus, filovirus, human papillomavirus, human T-cell lymphotropic virus, human retrovirus, cytomegalovirus, varicella-zoster virus, poliovirus, measles virus, rubella virus, mumps virus, adenovirus, enterovirus, rhinovirus, ebola virus, west nile virus and respiratory syncytial virus; especially infections caused by HIV, hepatitis, influenza virus, Japanese encephalitis virus, Epstein-Barr virus and respiratory syncytial virus. Bacterial infections include, but are not restricted to, those caused by Neisseria species, Meningococcal species, Haemophilus species, Salmonella species, Streptococcal species, Legionella species, Mycoplasma species, Bacillus species, Staphylococcus species, Chlamydia species, Actinomyces species, Anabaena species, Bacteroides species, Bdellovibrio species, Bordetella species, Borrella species, Campylobacter species, Caulobacter species, Chlorobium species, Chromatium species, Clostridium species, Corynebacterium species, Cytophaga species, Deinococcus species, Escherichia species, Francisella species, Helicobacter species, Haemophilus species, Hyphomicrobium species, Leptospira species, Usteria species, Micrococcus species, Myxococcus species, Nitrobacter species, Oscillatoria species, Prochloron species, Proteus species, Pseudomonas species, Rhodospirillum species, Rickettsia species, Shigella species, Spirillum species, Spirochaeta species, Streptomyces species, Thiobacillus species, Treponema species, Vibrio species, Yersinia species, Nocardia species and Mycobacterium species; especially infections caused by Neisseria species, Meningococcal species, Haemophilus species, Salmonella species, Streptococcal species, Legionella species and Mycobacterium species. Protozoan infections encompassed by the invention include, but are not restricted to, those caused by Plasmodium species, Leishmania species, Trypanosoma species, Toxoplasma species, Entamoeba species, and Giardia species. Helminth infections may include, but are not limited to, infections caused by Schistosoma species. Fungal infections contemplated by the present invention include, but are not limited to, infections caused by Histoplasma species and Candida species.

Suitable autoimmune disorders include, but are not limited to, autoimmune rheumatologic disorders (such as, for example, rheumatoid arthritis, Sjogren's syndrome, scleroderma, lupus such as systemic lupus erythematosus (SLE) and lupus nephritis, polymyositis-dermatomyositis, cryoglobulinemia, anti-phospholipid antibody syndrome and psoriatic arthritis), autoimmune gastrointestinal and liver disorders (such as, for example, inflammatory bowel diseases e.g., ulcerative colitis and Crohn's disease, autoimmune gastritis and pernicious anemia, autoimmune hepatitis, primary biliary cirrhosis, primary sclerosing cholangitis and celiac disease), vasculitis (such as, for example, anti-neutrophil cytoplasmic antibody (ANCA)-negative vasculitis and ANCA-associated vasculitis, including Churg-Strauss vasculitis, Wegener's granulomatosis and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as type I diabetes mellitis, Addison's disease and autoimmune thyroid disease (e.g., Graves' disease and thyroiditis)).

Suitable respiratory disorders include, but are not limited to, chronic obstructive pulmonary disease (CORD) or asthma especially allergic asthma.

In some embodiments, the methods further comprise detecting overexpression of a SETDB1 gene in a tumour sample obtained from the subject, wherein the tumour sample comprises the cancer stem cell tumour cells and optionally the non-cancer stem cell tumour cells, prior to administering the proteinaceous molecules of the invention to the subject.

In particular embodiments, any one of the methods described above involve the administration of one or more further active agents as described in Section 3 supra, such as an additional cancer therapy and/or an anti-infective agent, especially an additional cancer therapy.

The proteinaceous molecules of the invention, especially a proteinaceous molecule of any one of Formulas (I)-(IV), or any one of SEQ ID NOs: 2-6, or a variant proteinaceous molecule as described herein, are useful for inhibiting or reducing the acetylation of SETDB1. In some embodiments, the acetylation is catalyzed by an acetyltransferase; especially a histone acetyltransferase. In some embodiments, the histone acetyltransferase is GCN5, Hat1, ATF-2, Tip60, MOZ, MORF, HBO1, p300, CBP, SRC-1, ACTR, TIF-2, SRC-3, TAF1, TFIIIC and/or CLOCK; especially p300.

Thus, in a further aspect of the invention, there is provided a method of inhibiting the nuclear localization of SETDB1 in a subject, comprising administering a proteinaceous molecule as described herein. The invention also extends to a use of a proteinaceous molecule described herein for inhibiting or reducing the binding of a SETDB1 polypeptide with the binding of an importin polypeptide. In preferred embodiments, the proteinaceous molecules of the invention precent the importin polypeptide from transporting SETDB1 into the nuclear compartment.

The present invention also contemplates a method of producing a proteinaceous molecule that inhibits or reduces nuclear localization of a SETDB1 polypeptide, the method comprising:
a) contacting a cell with a proteinaceous molecule comprising, consisting or consisting essentially of an amino acid sequence corresponding to an NLS of SETDB1; and
b) detecting a reduction in or inhibition of the nuclear localization of the SETDB1 polypeptide in the cell relative to a normal or reference level of nuclear localization in the absence of the proteinaceous molecule.

In another aspect, the present invention provides a method of producing a SETDB1 bicyclic peptide mimetic that inhibits or reduces nuclear localization of SETDB1, wherein methylation of a methylation site of SETDB1 increases its nuclear localization in a cell, the method comprising:
a) contacting a cell with a SETDB1 bicyclic peptide mimetic comprising, consisting or consisting essentially of an amino acid sequence set forth in any one of Formula (I), or a bicyclic peptide of any one of Formulas (II)-(V); and
b) detecting a reduction in or inhibition of the nuclear localization of the nuclear localizable polypeptide in the cell relative to a normal or reference level of nuclear localization in the absence of the SETDB1 bicyclic peptide mimetic.

In some embodiments, the SETDB1 bicyclic peptide mimetic is distinguished from a native wild-type SETDB1 sequence at least by the addition of three cysteine residues.

A reduction in or inhibition of the nuclear localization of SETDB1 may be determined using standard techniques in the art, non-limiting examples of which include immunofluorescence, immunohistochemistry staining, chromatin immunoprecipitation (ChIP), ChIP-seq, chromatin accessibility assays such as DNase-seq, FAIRE-seq and ATAC-seq assays, such as that described in Satelli et al. (2016) Sci Rep, 6:28910; Bajetto, et al. (2000) Brain Research Protocols, 5(3): 273-281; and Sung, et al. (2014) BMC Cancer, 14:951, the entire contents of which are incorporated by reference.

A skilled person would be well aware of suitable assays used to evaluate the nuclear localization of a polypeptide, such as SETDB1, and to identify SETDB1 bicyclic peptide mimetics that inhibit or reduce the nuclear localization of a polypeptide. Screening for active agents according to the invention can be achieved by any suitable method. For example, the method may include contacting a cell expressing a polynucleotide corresponding to a gene that encodes the polypeptide of interest, such as SETDB1, with an agent suspected of having the inhibitory activity and screening for the inhibition or reduction of the level of the polypeptide of interest in the nucleus of the cell.

Alternatively, the inhibition of the functional activity of the polypeptide of interest or the lowering of the level of a transcript encoded by the polynucleotide, or the inhibition of the activity or expression of a downstream cellular target of the polypeptide or of the transcript, may be screened wherein the activity is related to nuclear localization of SETDB1. Detecting such inhibition may be achieved utilizing techniques including, but not limited to, ELISA, immunofluorescence, Western blots, immunoprecipitation, immunostaining, slot or dot blot assays, scintillation proximity assays, fluorescent immunoassays using antigen-binding molecule conjugates or antigen conjugates of fluorescent substances such as fluorescein or rhodamine, RIA, Ouchterlony double diffusion analysis, immunoassays employing an avidin-biotin or a streptavidin-biotin detection system, nucleic acid detection assays including reverse transcriptase polymerase chain reaction (RT-PCR), cell proliferation assays such as a WST-1 proliferation assay and immunoblot analysis of cells treated with SETDB1 Half-Way ChIP. The methylation of a polypeptide may be determined using an antibody directed to the methylated polypeptide, such as an antibody directed to a methylated lysine residue.

Active molecules may be further tested in the animal models to identify those molecules having the most potent in vivo effects. These molecules may serve as lead molecules for the further development of pharmaceuticals by, for example, subjecting the compounds to sequential modifications, molecular modeling and other routine procedures employed in rational drug design.

### 5. Methods of Detection and Prognosis/Prediction

In accordance with the present disclosure, nuclear localization of SETDB1 can be employed as a biomarker of response to therapy (e.g., immunotherapy). In some embodiments, nuclear localization of SETDB1 is determined by detecting co-localization of SETDB1 with a SETDB1 binding partner (e.g., ATF7IP or IMPα). Nuclear localization of SETDB1 is suitably assessed in SETDB1-expressing cells such as but not limited to, tumour cells. Representative SETDB1-expressing cell-containing subject samples include tissue samples such as solid tumours. In some embodiments, the sample is obtained prior to treatment with a therapy. In some embodiments, the tissue sample is formalin fixed and paraffin embedded, archival, fresh or frozen.

Presence and/or level/amount of a biomarker (e.g., SETDB1 (e.g., nuclear SETDB1, cytoplasmic SETDB1, etc.), or a complex comprising SETDB1 and a SETDB1 binding partner (e.g., ATF7IP, IMPα), also referred to herein collectively as “response-to-therapy biomarkers” or “RTT biomarkers”) at a cellular location (e.g., cytoplasm or nucleus) can be determined qualitatively and/or quantitatively based on any suitable criterion known in the art. In certain embodiments, presence and/or level/amount of an RTT biomarker at a cellular location in a first sample is increased or elevated as compared to presence/absence and/or level/amount at the cellular location in a second sample. In certain embodiments, presence/absence and/or level/amount of an RTT biomarker at a cellular location in a first sample is decreased or reduced as compared to presence and/or expression level/amount at the cellular location in a second sample.

In some embodiments of any of the methods, an elevated or higher level/amount refers to an overall increase of about any of 10%, 20%, 30%, 40%, 50%, 60%, 70%, 80%, 90%, 95%, 96%, 97%, 98%, 99% or greater, in the level/amount of an RTT biomarker at a cellular location, as detected for example by standard art known methods such as those described herein, as compared to the level/amount of the RTT biomarker at the cellular location in a reference sample, reference cell, reference tissue, control sample, control cell, or control tissue. In certain embodiments, the elevated or higher level/amount refers to an increase in level/amount of an RTT biomarker at the sample cellular location wherein the increase is at least about any of 1.2×, 1.3×, 1.4×, 1.5×, 1.75×, 2×, 3×, 4×, 5×, 6×, 7×, 8×, 9×, 10×, 25×, 50×, 75×, or 100× the level/amount of the RTT biomarker at the cellular location in a reference sample, reference cell, reference tissue, control sample, control cell, or control tissue. In some embodiments, an elevated or higher level/amount refers to an overall increase at a cellular location of greater than about 1.5-fold, about 1.75-fold, about-2 fold, about 2.25-fold, about 2.5-fold, about 2.75-fold, about 3.0-fold, or about 3.25-fold as compared to the cellular location in a reference sample, reference cell, reference tissue, control sample, control cell, control tissue, or internal control.

In some embodiments of any of the methods, an elevated or higher level/amount refers to a ratio of the level/amount of an RTT biomarker at a first cellular location (e.g., nuclear SETDB1) to the level/amount of the RTT biomarker at a second cellular location (e.g., cytoplasmic SETDB1), wherein the ratio is greater than any of about 0.55, 0.60, 0.65, 0.70, 0.75, 0.85, 0.90 or 0.95.

In some embodiments of any of the methods, an elevated or higher level/amount refers to higher level of an RTT biomarker in more than any of about 30%, 35%, 40%, 45%, 50%, 55%, 60%, 65%, 70%, 75%, 80%, 85%, 90%, or 95% of a subject's cells.

In some embodiments of any of the methods, a reduced or lower level/amount refers to an overall reduction of about any of 10%, 20%, 30%, 40%, 50%, 60%, 70%, 80%, 90%, 95%, 96%, 97%, 98%, 99% or greater, in the level of a RTT in a cellular location, as detected for example by standard art known methods such as those described herein, as compared to the level/amount in the cellular location in a reference sample, reference cell, reference tissue, control sample, control cell, or control tissue. In certain embodiments, a reduced or lower level/amount refers to a decrease in level/amount of a RTT biomarker in a cellular location in the sample wherein the decrease is at least about any of 0.9×, 0.8×, 0.7×, 0.6×, 0.5×, 0.4×, 0.3×, 0.2×, 0.1×, 0.05×, or 0.01× the level/amount of the RTT biomarker in the cellular location in a reference sample, reference cell, reference tissue, control sample, control cell, or control tissue.

Presence and/or level/amount of various RTT biomarkers in a sample can be analysed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), proteomics.

According to some embodiments, the presence and/or level/amount is measured by observing protein levels/amounts. In certain embodiments, the method comprises contacting the sample with an antibody to an RTT biomarker (e.g., anti-SETDB1 antibody), optionally in combination with an antibody to at least one SETDB1 binding partner (e.g., ATF7IP or IMPα) under conditions permissive for binding of the biomarker(s), and detecting whether a complex is formed between the antibody/antibodies and the biomarker(s).

Such method may be an in vitro or in vivo method. In some embodiments, one or more anti-RTT biomarker antibodies are used to select subjects eligible for treatment with a therapy (e.g., immunotherapy).

In certain embodiments, the presence and/or level/amount of biomarker proteins in a sample is examined using IHC and staining protocols. IHC staining of tissue sections has been shown to be a reliable method of determining or detecting presence or the level/amount of proteins in a sample, including the cellular localization of the proteins. In some embodiments, the level/amount of an RTT biomarker is determined using a method comprising: (a) performing IHC analysis of a sample (such as a tumour sample) with an antibody; and b) determining the level/amount of the biomarker at a cellular location (e.g., nuclear or cytoplasm) in the sample. In some embodiments, IHC staining intensity is determined relative to a reference. In some embodiments, the reference is a reference value. In some embodiments, the reference is a reference sample (e.g., control cell line staining sample or tissue sample from a non-cancerous subject).

In some embodiments, the presence and/or level/amount of an RTT biomarker is evaluated on a tumour or tumour sample. As used herein, a tumour or tumour sample may encompass part or all of the tumour area occupied by tumour cells. In some embodiments, a tumour or tumour sample may further encompass tumour area occupied by tumour associated intratumoural cells and/or tumour associated stroma (e.g., contiguous peri-tumoural desmoplastic stroma). Tumour associated intratumoural cells and/or tumour associated stroma may include areas of immune infiltrates (e.g., tumour infiltrating immune cells as described herein) immediately adjacent to and/or contiguous with the main tumour mass. In some embodiments, RTT biomarker expression is evaluated on tumour cells.

In alternative methods, the sample may be contacted with an antibody specific for an RTT biomarker under conditions sufficient for an antibody-biomarker complex to form, and then detecting the complex. The presence or level/amount of the biomarker may be detected in a number of ways, such as by Western blotting and ELISA procedures for assaying a wide variety of tissues and samples, including plasma or serum. A wide range of immunoassay techniques using such an assay format are available (see, e.g., U.S. Pat. Nos. 4,016,043, 4,424,279 and 4,018,653). These include both single-site and two-site or “sandwich” assays of the non-competitive types, as well as in the traditional competitive binding assays. These assays also include direct binding of a labelled antibody to a target biomarker.

In certain embodiments, the samples are normalized for both differences in the amount of the biomarker assayed and variability in the quality of the samples used, and variability between assay runs. Such normalization may be accomplished by detecting and incorporating the expression of certain normalizing biomarkers, including expression products of well-known housekeeping genes. Alternatively, normalization can be based on the mean or median signal of all of the assayed proteins or a large subset thereof (global normalization approach). On a protein-by protein basis, measured normalized amount of a subject tumour protein is compared to the amount found in a reference set. Normalized levels for each protein per tested tumour per subject can be expressed as a percentage of the expression level measured in the reference set. The presence and/or level/amount measured in a particular subject sample to be analysed will fall at some percentile within this range, which can be determined by methods well known in the art.

In some embodiments, the sample is a clinical sample. In other embodiments, the sample is used in a diagnostic assay. In some embodiments, the sample is obtained from a primary or metastatic tumour. Tissue biopsy is often used to obtain a representative piece of tumour tissue. Alternatively, tumour cells can be obtained indirectly in the form of tissues or fluids that are known or thought to contain the tumour cells of interest. For instance, samples of lung cancer lesions may be obtained by resection, bronchoscopy, fine needle aspiration, bronchial brushings, or from sputum, pleural fluid or blood. Proteins can be detected from cancer or tumour tissue or from other body samples such as urine, sputum, serum or plasma. Cancer cells may be sloughed off from cancer lesions and appear in such body samples. By screening such body samples, a simple early diagnosis can be achieved for these cancers. In addition, the response to a therapy can be monitored more easily by testing such body samples for RTT biomarkers.

In certain embodiments, a reference sample, reference cell, reference tissue, control sample, control cell, or control tissue is a single sample or combined multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic or resistant to treatment with a therapy.

In certain embodiments, a reference sample, reference cell, reference tissue, control sample, control cell, or control tissue is a combination of multiple samples from one or more healthy individuals who are not the subject or individual. In certain embodiments, a reference sample, reference cell, reference tissue, control sample, control cell, or control tissue is a combination of multiple samples from one or more individuals with a disease (e.g., cancer) who are not the subject or individual. In certain embodiments, a reference sample, reference cell, reference tissue, control sample, control cell, or control tissue is pooled samples from normal tissues or biological fluids such as blood from one or more individuals who are not the subject or individual. In certain embodiments, a reference sample, reference cell, reference tissue, control sample, control cell, or control tissue is pooled samples from tumour tissues or pooled blood samples from one or more individuals with a disease (e.g., cancer) who are not the subject or individual.

In some embodiments, the sample is a tissue sample from the individual. In some embodiments, the tissue sample is a tumour tissue sample (e.g., biopsy tissue). In some embodiments, the tissue sample is lung tissue. In some embodiments, the tissue sample is renal tissue. In some embodiments, the tissue sample is skin tissue. In some embodiments, the tissue sample is pancreatic tissue. In some embodiments, the tissue sample is gastric tissue. In some embodiments, the tissue sample is bladder tissue. In some embodiments, the tissue sample is esophageal tissue. In some embodiments, the tissue sample is mesothelial tissue. In some embodiments, the tissue sample is breast tissue. In some embodiments, the tissue sample is thyroid tissue. In some embodiments, the tissue sample is colorectal tissue. In some embodiments, the tissue sample is head and neck tissue. In some embodiments, the tissue sample is osteosarcoma tissue. In some embodiments, the tissue sample is prostate tissue. In some embodiments, the tissue sample is ovarian tissue, HCC (liver), blood cells, lymph nodes, and/or bone/bone marrow tissue. In some embodiments, the tissue sample is colon tissue. In some embodiments, the tissue sample is endometrial tissue. In some embodiments, the tissue sample is brain tissue (e.g., glioblastoma, neuroblastoma, and so forth).

In some embodiments, a tumour tissue sample (the term “tumour sample” is used interchangeably herein) may encompass part or all of the tumour area occupied by tumour cells. In some embodiments, a tumour or tumour sample may further encompass tumour area occupied by tumour associated intratumoural cells and/or tumour associated stroma (e.g., contiguous peri-tumoural desmoplastic stroma). Tumour associated intratumoural cells and/or tumour associated stroma may include areas of immune infiltrates (e.g., tumour infiltrating immune cells as described herein) immediately adjacent to and/or contiguous with the main tumour mass.

In some embodiments, tumour cell staining is expressed as the percentage of all tumour cells showing nuclear staining of any intensity. Infiltrating immune cell staining may be expressed as the percent of the total tumour area occupied by immune cells that show staining of any intensity. The total tumour area encompasses the malignant cells as well as tumour-associated stroma, including areas of immune infiltrates immediately adjacent to and contiguous with the main tumour mass. In addition, infiltrating immune cell staining may be expressed as the percent of all tumour infiltrating immune cells.

In some embodiments of any of the methods, the disease is a tumour. In some embodiments, the tumour is a malignant cancerous tumour (i.e., cancer). In some embodiments, the tumour and/or cancer is a solid tumour or a non-solid or soft tissue tumour. Examples of soft tissue tumours include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia) or lymphoma (e.g., non-Hodgkin's lymphoma, cutaneous T-cell lymphoma, or Hodgkin's disease). A solid tumour includes any cancer of body tissues other than blood, bone marrow, or the lymphatic system. Solid tumours can be further divided into those of epithelial cell origin and those of non-epithelial cell origin. Examples of epithelial cell solid tumours include tumours of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelial carcinoma, dysplastic urothelial carcinoma, transitional cell carcinoma), bladder, and skin. Solid tumours of non-epithelial origin include sarcomas, brain tumours, and bone tumours. In some embodiments, the cancer is non-small cell lung cancer (NSCLC). In some embodiments, the cancer is second-line or third-line locally advanced or metastatic non-small cell lung cancer. In some embodiments, the cancer is adenocarcinoma. In some embodiments, the cancer is squamous cell carcinoma. In some embodiments, the cancer is non-small cell lung cancer (NSCLC), glioblastoma, neuroblastoma, melanoma, breast carcinoma (e.g., triple-negative breast cancer), gastric cancer, colorectal cancer (CRC), or hepatocellular carcinoma. In some embodiments, the cancer is a primary tumour. In some embodiments, the cancer is a metastatic tumour at a second site derived from any of the above types of cancer.

In some embodiments, an RTT biomarker is detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, and combinations thereof. In some embodiments, the RTT biomarker is detected in blood samples. In some embodiments, the RTT biomarker is detected in circulating tumour cells in blood samples. Any suitable method to isolate/enrich such population of cells may be used including, but not limited to, cell sorting. In some embodiments, the level/amount of nuclear SETDB1 is reduced in samples from individuals that respond to treatment with a therapy, suitably an immunotherapy (e.g., one that comprises an anti-immune checkpoint molecule antibody such as an anti-PD-1 antagonist antibody). In some embodiments, the level/amount of nuclear SETDB1 is elevated in samples from individuals that do not respond or respond weakly to treatment with a therapy, suitably an immunotherapy (e.g., one that comprises an anti-immune checkpoint molecule antibody such as an anti-PD-1 antagonist antibody). In some embodiments, the level/amount of extranuclear SETDB1 is reduced in samples from individuals that do not respond or weakly respond to treatment with a therapy, suitably an immunotherapy (e.g., one that comprises an anti-immune checkpoint molecule antibody such as an anti-PD-1 antagonist antibody). In some embodiments, the level/amount of extranuclear SETDB1 is elevated in samples from individuals that respond to treatment with a therapy, suitably an immunotherapy (e.g., one that comprises an anti-immune checkpoint molecule antibody such an anti-PD-1 antagonist antibody).

Also provided herein are predictive/prognostic methods and kits that are based on the determination that SETDB1 co-localizes in the nucleus with a nuclear binding partner of SETDB1 (e.g., ATF7IP) and that this co-localization contributes at least in part to resistance or non-responsiveness to therapy (e.g., immunotherapy) and/or disease status (e.g., severity or progression of disease). These methods suitably comprise: (i) obtaining a sample from a subject, wherein the sample comprises a SETDB1-expressing cell (e.g., a tumour cell); (ii) contacting the sample with a first antigen-binding molecule that binds to SETDB1 in the sample and a second antigen-binding molecule that binds to the SETDB1-binding partner in the sample; and (iii) detecting localization of the first and second antigen-binding molecule(s) in the nucleus of the SETDB1-expressing cell, wherein localization of the first and second antigen-binding molecules in the nucleus of the SETDB1-expressing cell is indicative that the SETDB1-expressing cell has increased likelihood of resistance to the therapy, that the subject is a likely non-responder to the therapy, that the subject is selected for not treating with the therapy, and/or that the treatment outcome for the subject is predicted to be a likely negative treatment outcome.

Localization of SETDB1 and the nuclear binding partner of SETDB1 in the nucleus of a SETDB1-expressing cell may be performed using any suitable localization technique, e.g., by IHC, typically using an anti-SETDB1 antibody that has a different detectable moiety or label than an anti-SETDB1-binding partner antibody. In some embodiments, spatial proximity assays (also referred to as “proximity assays”) are employed, which can be used to assess the formation of a complex between the SETDB1 and the nuclear binding partner of SETDB1. Proximity assays rely on the principle of “proximity probing”, wherein an analyte, typically an antigen, is detected by the coincident binding of multiple (i.e., two or more, generally two, three or four) binding agents or probes, which when brought into proximity by binding to the analyte (hence “proximity probes”) allow a signal to be generated.

In some embodiments, at least one of the proximity probes comprises a nucleic acid domain (or moiety) linked to the analyte-binding domain (or moiety) of the probe, and generation of the signal involves an interaction between the nucleic acid moieties and/or a further functional moiety which is carried by the other probe(s). Thus, signal generation is dependent on an interaction between the probes (more particularly by the nucleic acid or other functional moieties/domains carried by them) and hence only occurs when both the necessary two (or more) probes have bound to the analyte, thereby lending improved specificity to the detection system.

The concept of proximity probing has been developed in recent years and many assays based on this principle are now well known in the art.

Proximity assays are typically used to assess whether two particular proteins or portions thereof are in close proximity, e.g., proteins that are bound to each other, fusion proteins, and/or proteins that are positioned in close proximity. One such assay, known as proximity ligation assay (PLA), and which is used in some embodiments of the present disclosure, features two antibodies (raised in different species) bound to the targets of interest (see, Nature Methods 3, 995-1000 (2006)). PLA probes, which are species-specific secondary antibodies with a unique oligonucleotide strand attached, are then bound to the appropriate primary antibodies. In the case of the targets being in close proximity, the oligonucleotide strands of the PLA probes can interact with additional ssDNA and DNA ligase such they can be circulated and amplified via rolling circle amplification (RCA). When highly processive DNA polymerases such as Phi29 DNA polymerase is used, the circular DNA template can be replicated hundreds to thousands of times longer and as a result producing ssDNA molecules from hundreds of nanometers to microns in length (see, Angewandte Chemie International Edition, 2008, 47, 6330-6337). After the amplification, the replicated DNA can be detected via detection systems. Thus, a visible signal is indicative that the targets of interest are in close proximity. These assays feature the use of several DNA-antibody conjugates as well as enzymes such as DNA ligase and DNA polymerase.

In other embodiments, a dual binders (DB) assay is employed, which utilizes a bi-specific detection agent consisting of two Fab fragments with fast off-rate kinetics joined by a flexible linker (Van dieck et al., 2014 Chemistry & Biology Vol. 21 (3): 357-368). In principle, because the dual binders comprise Fab fragments with fast off-rate kinetics, the dual binders are washed off if only one of the Fab fragments is bound to its epitope (simultaneous cooperative binding of both Fab fragments of the dual binder prevents dissociation of the dual binder and leads to positive staining/visibility).

According to another approach disclosed in International PCT Publication No. WO2014/139980, which is encompassed in the practice of the present disclosure, proximity assays and tools are described, which employ a biotin ligase substrate and an enzyme to perform a proximity assay. The method provides detection of target molecules and proximity while maintaining the cellular context of the sample. The use of biotin ligase such as an enzyme from E. coli and peptide substrate such as amino-acid substrate for that enzyme provides for a sensitive and specific detection of protein-protein interactions in FFPE samples. Because biotin ligase can efficiently biotinylate appropriate peptide substrate in the presence of biotin and the reaction can only occur when the enzyme makes physical contact with the peptide substrate, biotin ligase and the substrate can be separately conjugated to two antibodies that recognize targets of interest respectively.

In some embodiments, the level/amount of one or more biomarker proteins and/or their cellular location/distribution may be compared to a reference which may include a sample from a subject not receiving a therapy (e.g., an immunotherapy). In some embodiments, a reference may include a sample from the same subject before receiving a therapy (e.g., an immunotherapy). In some embodiments, a reference may include a reference value from one or more samples of other subjects receiving a therapy (e.g., an immunotherapy). For example, a population of subjects may be treated, and a mean, average, or median value for level/amount of the at least one RTT biomarker and/or their cellular location/distribution may be generated from the population as a whole. A set of samples obtained from diseases having a shared characteristic (e.g., the same cancer type and/or stage, or exposure to a common therapy) may be studied from a population, such as with a clinical outcome study. This set may be used to derive a reference (e.g., a reference number) to which a subject's sample may be compared.

Certain aspects of the present disclosure relate to measurement of the level/amount of one or more RTT biomarkers in a sample. In some embodiments, a sample may include cancer cells. In some embodiments, the sample may be a peripheral blood sample (e.g., from a subject having a tumour). In some embodiments, the sample is a tumour sample. A tumour sample may include cancer cells, lymphocytes, leukocytes, stroma, blood vessels, connective tissue, basal lamina, and any other cell type in association with the tumour. In some embodiments, the sample is a tumour tissue sample containing tumour-infiltrating leukocytes. In some embodiments, the sample may be processed to separate or isolate one or more cell types (e.g., leukocytes). In some embodiments, the sample may be used without separating or isolating cell types.

A tumour sample may be obtained from a subject by any method known in the art, including without limitation a biopsy, endoscopy, or surgical procedure. In some embodiments, a tumour sample may be prepared by methods such as freezing, fixation (e.g., by using formalin or a similar fixative), and/or embedding in paraffin wax. In some embodiments, a tumour sample may be sectioned. In some embodiments, a fresh tumour sample (i.e., one that has not been prepared by the methods described above) may be used. In some embodiments, a tumour sample may be prepared by incubation in a solution to preserve mRNA and/or protein integrity.

In some embodiments, responsiveness to therapy may refer to any one or more of: extending survival (including overall survival and progression free survival); resulting in an objective response (including a complete response or a partial response); or improving signs or symptoms of cancer. In some embodiments, responsiveness may refer to improvement of one or more factors according to the published set of RECIST guidelines for determining the status of a tumour in a cancer subject, i.e., responding, stabilizing, or progressing. For a more detailed discussion of these guidelines, see, Eisenhauer et al. (2009 Eur. J. Cancer 45: 228-47), Topalian et al. (2012 N Engl. J. Med. 366:2443-54), Wolchok et al. (2009 Clin. Can. Res. 15:7412-20) and Therasse et al. (2000 J. Natl. Cancer Inst. 92:205-16). A responsive subject may refer to a subject whose cancer(s) show improvement, e.g., according to one or more factors based on RECIST criteria. A non-responsive subject may refer to a subject whose cancer(s) do not show improvement, e.g., according to one or more factors based on RECIST criteria.

In some embodiments, conventional response criteria may not be adequate to characterize the activity of an anti-cancer therapy, which can produce delayed responses that may be preceded by initial apparent radiological progression, including the appearance of new lesions. Therefore, modified response criteria have been developed that account for the possible appearance of new lesions and allow radiological progression to be confirmed at a subsequent assessment. Accordingly, in some embodiments, responsiveness may refer to improvement of one of more factors according to immune-related response criteria (irRC). See, e.g., Wolchok et al. (2009, supra). In some embodiments, new lesions are added into the defined tumour burden and followed, e.g., for radiological progression at a subsequent assessment. In some embodiments, presence of non-target lesions is included in assessment of complete response and not included in assessment of radiological progression. In some embodiments, radiological progression may be determined only on the basis of measurable disease and/or may be confirmed by a consecutive assessment around 4 weeks from the date first documented.

### 6. Subject Classification and Treatment Management

The present disclosure extends to methods of selecting or identifying individuals who are appropriate candidates for treatment with a therapy (e.g., an immunotherapy) for treatment of a disease (e.g., cancer). Such individuals include subjects that are predicted to be responsive to the therapy and thus have an increased likelihood of benefiting from administration of the therapy relative to other subjects having different characteristic(s) (e.g., non-responsiveness to the therapy). In certain embodiments an appropriate candidate is one who is reasonably likely to benefit from treatment or at least sufficiently likely to benefit as to justify administering the treatment in view of its risks and side effects. The disclosure also encompasses methods of selecting or identifying individuals who are not appropriate candidates for treatment with a therapy (e.g., an immunotherapy) for treatment of a disease (e.g., cancer). Such individuals include subjects that are predicted to be non-responsive or weakly responsive to the therapy and thus have a decreased likelihood of benefiting from administration of the therapy relative to other subjects having different characteristic(s) (e.g., responsiveness to the therapy), or a low or substantially no likelihood of benefiting from such treatment, such that it may be desirable to use a different or additional treatment. In some embodiments, whether a subject is an appropriate candidate for therapy with a therapy is determined based on an assay of at least one RTT biomarker in a sample obtained from a subject, as described herein.

In some aspects described herein are methods of determining, for example based on an assay of at least one RTT biomarker, the likelihood that a subject in need of treatment of a disease (e.g., cancer) will respond to treatment with a therapy (e.g., immunotherapy) and/or of identifying and/or selecting a subject to receive such treatment. In specific embodiments, the therapy is an immunotherapy, suitably with an anti-immune checkpoint inhibitor. The phrase “treatment with an immune checkpoint inhibitor”, also referred to as “immune checkpoint inhibitor treatment”, “therapy with an immune checkpoint inhibitor”, or “immune checkpoint inhibitor therapy”, encompasses embodiments pertaining to treatment with a single immune checkpoint inhibitor and embodiments pertaining to treatment with two or more immune checkpoint inhibitors in combination. In some embodiments immune checkpoint inhibitor treatment comprises inhibiting two or more different immune checkpoint pathways using a single agent or using two or more separate agents

### 7. Kits

In other embodiments of the invention, therapeutic kits are provided comprising a SETDB1 bicycle peptide mimetic and an anticancer spray. In some embodiments, the therapeutic kits further comprise a package insert comprising instructional material for administering concurrently the SETDB1 bicycle peptide mimetic and anti-cancer agent to treat a T cell dysfunctional disorder, or to enhance immune function (e.g., immune effector function, T cell function etc.) in an individual having cancer, or to treat or delay cancer progression, or to treat infection in an individual. In some embodiments, the anti-cancer agent comprises a chemotherapeutic agent (e.g., an agent that targets rapidly dividing cells and/or disrupt the cell cycle or cell division, representative examples of which include cytotoxic compounds such as a taxane).

In some embodiments, the SETDB1 peptide described above and optionally chemotherapeutic agents are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The kits may further include other materials desirable from a commercial and user standpoint, including other buffers, diluents, filters, needles, syringes, and package inserts with instructional material for use. In some embodiments, the kits further include one or more of other agents (e.g., a chemotherapeutic agent, and anti-neoplastic agent). Suitable containers for the one or more agent include, for example, bottles, vials, bags and syringes.

In other embodiments of the invention, diagnostic kits are provided for determining expression of biomarkers, which include reagents that allow detection and/or quantification of the biomarkers. Such reagents include, for example, compounds or materials, or sets of compounds or materials, which allow quantification of the biomarkers. In specific embodiments, the compounds, materials or sets of compounds or materials permit determining the expression level of a gene (e.g., T-cell function biomarker gene), including without limitation the extraction of RNA material, the determination of the level of a corresponding RNA, etc., primers for the synthesis of a corresponding cDNA, primers for amplification of DNA, and/or probes capable of specifically hybridizing with the RNAs (or the corresponding cDNAs) encoded by the genes, TaqMan probes, proximity assay probes, ligases, antibodies etc.

The kits may also optionally include appropriate reagents for detection of labels, positive and negative controls, washing solutions, blotting membranes, microtiter plates, dilution buffers and the like. For example, a nucleic acid-based detection kit may include (i) a T-cell function biomarker polynucleotide (which may be used as a positive control), (ii) a primer or probe that specifically hybridizes to a biomarker polynucleotide. Also included may be enzymes suitable for amplifying nucleic acids including various polymerases (reverse transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) a biomarker polypeptide (which may be used as a positive control), (ii) an antibody that binds specifically to a biomarker polypeptide. The kit can also feature various devices (e.g., one or more) and reagents (e.g., one or more) for performing one of the assays described herein; and/or printed instructional material for using the kit to quantify the expression of a T-cell function biomarker gene. The reagents described herein, which may be optionally associated with detectable labels, can be presented in the format of a microfluidics card, a chip or chamber, a microarray or a kit adapted for use with the assays described in the examples or below, e.g., RT-PCR or Q PCR techniques described herein.

In another aspect the present invention extends to kits for determining level/amounts and/or cellular localization of RTT biomarkers disclosed herein, which include reagents that allow detection and/or quantification of the biomarkers. Such reagents include, for example, compounds or materials, or sets of compounds or materials, which allow quantification of the biomarkers. In specific embodiments, the compounds, materials or sets of compounds or materials permit determining the level of a biomarkers, including without limitation the extraction of RNA material, the determination of the level of a corresponding RNA, etc., primers for the synthesis of a corresponding cDNA, primers for amplification of DNA, and/or probes capable of specifically hybridizing with the RNAs (or the corresponding cDNAs) encoded by the genes, TaqMan probes, proximity assay probes, ligases, antibodies etc.

The kits may also optionally include appropriate reagents for detection of labels, positive and negative controls, washing solutions, blotting membranes, microtiter plates, dilution buffers and the like. For example, a protein-based detection kit may include (i) at least one SETDB1 polypeptide (which may be used as a positive control); (ii) one or more antigen-binding molecules that bind specifically to a SETDB1 polypeptide; and/or (iii) at least one nuclear binding partner of SETDB1 (e.g., ATF7IP, IMPα). The antigen-binding molecules are suitably detectably labelled. The kit can also feature various devices (e.g., one or more) and reagents (e.g., one or more) for performing one of the assays described herein; and/or printed instructional material for using the kit to quantify the level/amount of an RTT biomarker. The reagents described herein, which may be optionally associated with detectable labels, can be presented in the format of a microfluidics card, a chip or chamber, a microarray or a kit adapted for use with the assays described herein.

Materials suitable for packing the components of the diagnostic kits may include crystal, plastic (polyethylene, polypropylene, polycarbonate and the like), bottles, vials, paper, envelopes and the like. Additionally, the kits of the invention can contain instructional material for the simultaneous, sequential or separate use of the different components contained in the kit. The instructional material can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain internet addresses that provide the instructional material.

In order that the invention may be readily understood and put into practical effect, particular preferred embodiments will now be described by way of the following non-limiting examples.

### EXAMPLES

### Example 1

### Prototypic Linear SETDB1 Peptide Inhibitors

### 7.1 SETDB1 Localised to the Cell Nucleus of Cancer Cells.

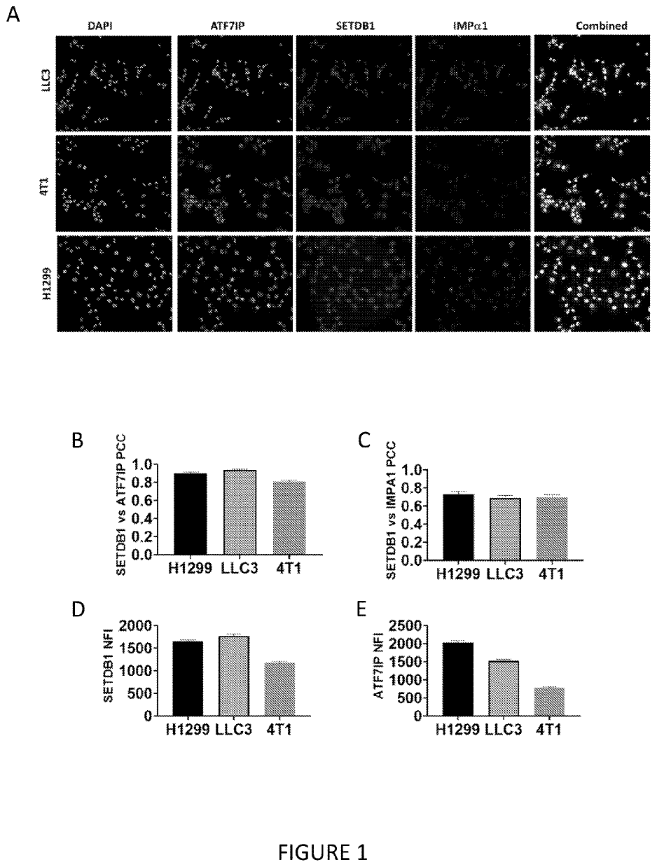

The present inventors discovered that three cancer cell lines (H1299, 4T1, and LLC3) displayed significant nuclear expression of SETDB1 and ATF7IP. In addition, there was significant co-localization of SETDB1 and ATF7IP or SETDB1 and IMPα1 in all three cancer cell lines tested (FIG. 1). Nuclear localization of epigenetic enzymes are a feature of aggressive metastatic cancers. Therefore, the present inventor hypothesized that targeting the nuclear axis of the epigenetic enzyme SETDB1 and its major nuclear interacting partner ATF7IP, would directly inhibit the mesenchymal, therapeutic-resistant signature. To do this, the nuclear localization sequence of SETDB1 was targeted.

### 7.2 Generation of SETDB1 NLS Mimetic Peptide Inhibitors.

In light of the above data, novel SETDB1 NLS mimetic peptide inhibitors were developed, based on a newly identified nuclear localisation sequence (NLS) motif of SETDB1. Notably, the NLS region of SETDB1 is highly conserved across species.

| | NLS Region of SETDB1 | SEQ ID NO: |
|---|---|---|
| Gorilla | GKKRTKTWHKGTLIAIQTVGPGKKYKV | 59 |
| Human | GKKRTKTWHKGTLIAIQTVGPGKKYKV | 60 |
| Rat | GKKRTKTWHKGTLIAIQTVGLGKKYKV | 61 |
| Canine | GKKRTKTWHKGTLIAIQTVGPGKKYKV | 62 |
| Macaque | GKKRTKTWHKGTLIAIQTVGPGKKYKV | 63 |

Two SETDB1 nuclear localisation sequence (NLS) peptide mimetics were generated, (i) peptide 047_wt, corresponding to residues of the wild-type full-length human SETDB1 amino acid sequence (including the native alanine residue at the 15^{th} position), and (ii) peptide 047_A15P (as set forth in SEQ ID NO: 1) which contains a single amino acid substitution at the 15^{th} position (alanine to proline). This amino acid substitution was performed as it was hypothesized that proline may enhance peptide stability and the interactions with its targets. Accordingly, the novel peptide sequences have the following amino acid sequences:

| | |
|---|---|
| Peptide 047_wt | Myristoyl-GKKRTKTWHKGTLIAIQTVGPG |
| | KKYKV |
| | [SEQ ID NO: 64] |
| Peptide 047_A15P | Myristoyl-GKKRTKTWHKGTLIPIQTVGPG |
| | KKYKV |
| | [SEQ ID NO: 65] |

### 7.3 SETDB1 NLS Peptide Inhibitors Abrogate Cell Proliferation.

The effect of the candidate peptide inhibitors (peptide 047_A15P and peptide 047_wt) on the cell proliferation of the TNBC cancer cell line MDA-MB-231 and melanoma cancer cell line RPMI-7951 was investigated.

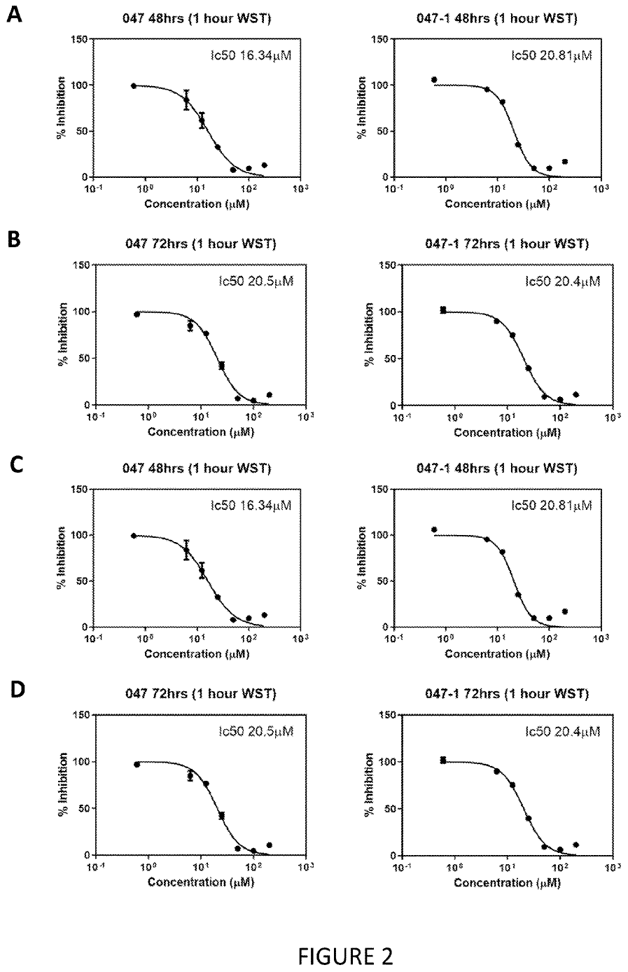

Both linear SETDB1 peptide inhibitors (i.e., peptide 047_A15P and peptide 047_wt) are able to abrogate proliferation of the MDA-MB-231 TNBC metastatic cancer cell line with an IC of ~20 mM or below (FIG. 2A,B). Similarly, the linear SETDB1 peptide inhibitors are able to abrogate proliferation of the RPMI-7951 metastatic melanoma cancer cell lines (FIG. 2C,D).

### 7.4 SETDB1 NLS Peptide Inhibitors Reduce Metastatic Markers.

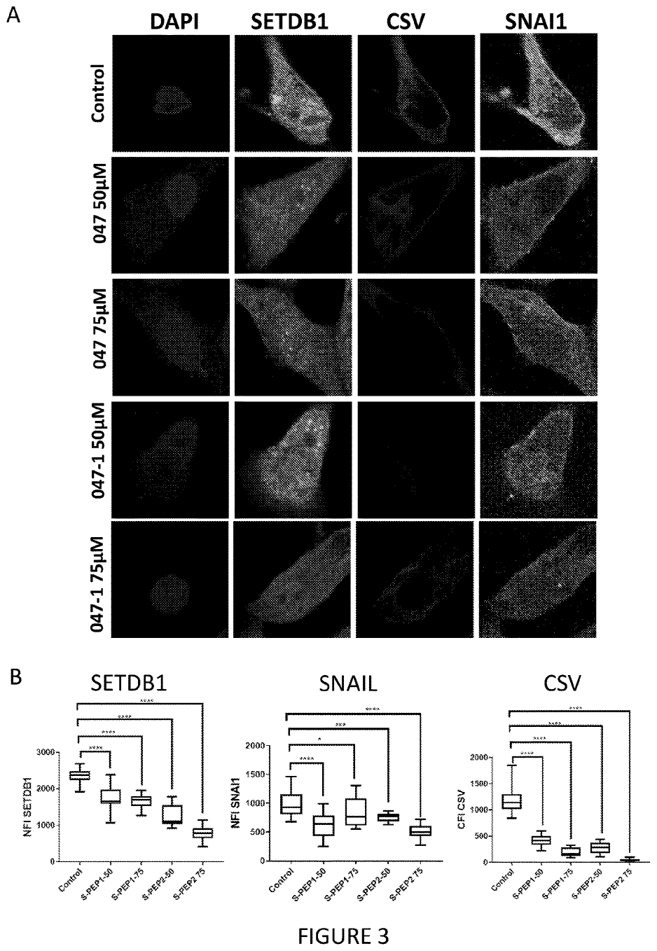

Next, the present inventors sought to probe the effect of inhibiting SETDB1 nuclear translocation on protein markers for mesenchymal, metastatic cancer. In particular, CSV, SNAIL are both markers of cancer that is metastatic and invasive. The direct target, SETDB1, was also examined. Immunofluorescent analysis of protein expression in MDA-MB-231 TNBC cancer cells demonstrated that both linear peptide inhibitors and at different concentrations induced a significant reduction in the level of the mesenchymal metastatic markers CSV, SNAIL as well as nuclear SETDB1 expression (FIG. 3). These data indicate that targeting and inhibiting the nuclear axis of SETDB1 directly impacts the mesenchymal, metastatic markers that mediate cancer progression and metastatic spread.

### Materials & Methods

### IFA Microscopy

To examine the signature of SETDB1, ATF7IP and IMPα1, in LLC3 (lung cancer), H1299 (lung cancer), and 4T1 (TNBC) cell lines, cells were permeabilised by incubating with 0.5% Triton X-100 for 15 min, blocked with 1% BSA in PBS and were probed with either ATF7IP, IMPα1, SETDB1. Antibodies were visualized with a donkey anti-rabbit AF 488, anti-mouse AF 568, donkey anti-goat 647. Cover slips were mounted on glass microscope slides with ProLong Glass Antifade reagent (Life Technologies). Protein targets were localised by digital pathology laser scanning microscopy. Single 0.5 μm sections were obtained using a ASI Digital pathology microscope using 100× oil immersion lens running ASI software. The final image was obtained by averaging four sequential images of the same section. Digital images were analyzed using automated ASI software (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. Digital images were also analysed using ImageJ software (ImageJ, NIH, Bethesda, MD, USA) to determine the total cell fluorescence or cell surface only fluorescence for non-permeabilised cells. Digital images were analysed using ImageJ software (ImageJ, NIH, Bethesda, MD, USA) to determine either the Total Nuclear Fluorescent Intensity (TNFI), the Total Cytoplasmic Fluorescent Intensity (TCFI). ImageJ software with automatic thresholding and manual selection of regions of interest (ROIs) specific for cell nuclei was used to calculate the Pearson's co-efficient correlation (PCC) for each pair of antibodies. PCC values range from: −1=inverse of co-localisation, 0=no co-localisation, +1=perfect co-localisation. The Mann-Whitney nonparametric test (GraphPad Prism, GraphPad Software, San Diego, CA) was used to determine significant differences between datasets.

This methodology was also used to examine the signature of SETDB1, CSV, and SNAIL in MDA-MB231 TNBC cells.

### Cell Culture Methods

All breast cancer cell lines used were sourced from ATCC. MDA-MB-231 or MDB-MB-231-Br cell lines were maintained and cultured in DMEM (Invitrogen) supplemented with 10% FBS, 2 mM L-glutamine, and 1% PSN. MCF-7 cells were stimulated with 1.29 ng/ml phorbol 12-myristate 13-acetate (PMA) (Sigma-Aldrich) or 5 ng/ml recombinant TGF-β1 (R&D Systems) for 60 hours. For inhibitor studies, 2×10⁵ cells were seeded in 2 mL complete media in 6 well plates and incubated overnight at 37° C./5% CO₂. Cells were treated with SETDB1 linear peptides. For microscopy studies, 4×10⁴ cells were seeded on coverslips and treated with inhibitors as described above. At each timepoint, coverslips were washed, fixed with 3.7% formaldehyde (Sigma) and stored at 4° C. until processing.

### Cell Viability Assay-MDA-MB2321 and RPMI-7951

MDA-MB-231 or RPMI-7951 cell lines were seeded at 4×10³ cells/well into a 96-well flat bottom tissue culture plate in a final volume of 100 μL and left to adhere for 24 hours at 37° C. and 5% CO₂. Media was then removed and cells were treated with SETDB1 peptide inhibitors at the following final concentrations: 200, 100, 50, 25, 12.5 and 6.25 μM for 48 or 72 hrs. Following inhibition, media was removed and replaced with 100 μl/well of WST-1 cell proliferation reagent (Sigma-Aldrich, Cat #11644807001) at 1:10 final dilution. Absorbance was recorded at 450 nm after 1 hour using a microplate spectrophotometer (with 30 sec mix time). IC50 values for inhibitors were calculated from proliferation (%) and log concentration using graphpad prism software. Data represent an independent experiment performed in triplicate, results are graphed as mean+/−standard error (SE).

### Example 2

### SETDB1 Bicyclic Peptide Inhibitors

### Generation of SETDB1 Bicyclic Peptide Inhibitors.

Based on the effectiveness of the linear peptide inhibitor compounds, the present inventors next embarked on a bicyclic peptide design approach for progression to in vivo work. Three bicyclic peptide inhibitor constructs based on the original linear sequence of the SETDB1 NLS region were proposed. The bicyclic peptides were originally designed for use with a 1,3,5-(TribroMoMethyl)benzene (TBMB) molecular backbone, and the sequences of the three constructs are shown in Table 4. The NLS motif (i.e., GKKRTKT) and other critical residues are underlined. In order to make interactions with the proposed molecular backbone, three cysteine residues need to be introduced into the peptide sequence. The newly introduced cysteines are shown in Table 4 using bold typeface. The present inventors also designed a D-isomer version of MSETC (“QIMR2-D”), and a D-isomer retero-inverso version of MSETC (“MSETC-D-R”).

TABLE 4

| Candidate | Amino Acid Sequence | SEQ ID NO | Backbone |
|---|---|---|---|
| MSETC | Myr-CGKKRTKTWHCKGTLIAIQTVGPGCKKYKV | 66 | TBMB |
| MSETC | Myr-CGKKRTKTWHCKGTLIAIQTVGPGCKKYKV | 66 | TBAB |
| QIMRB-3 | Myr-CGKKRTKTWHCKGTLIAIQCTVGPGKKYKV | 67 | TBMB |
| MSETC-D | Myr-GckkrtktwhckGtliaiqtvGpGckkykv | 68 | TBMB |
| MSETC-D-R | Myr-vkykkcGpgvtqisiltGkchwtktrkkcG | 69 | TBMB |

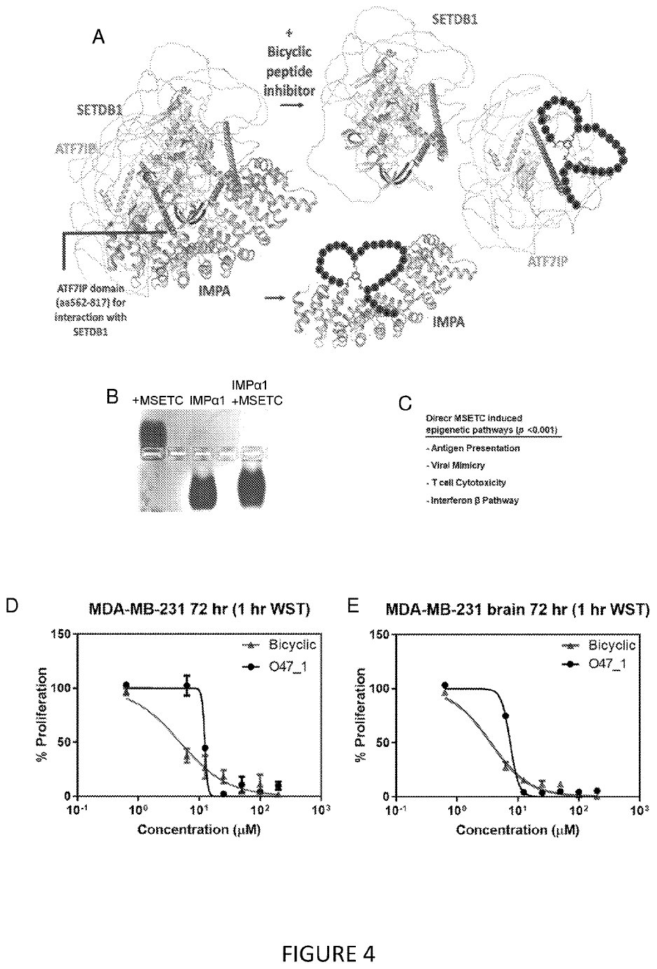

The present inventors considered that the placement of the of the cysteine linker regions in QIMRB-3 would likely change the conformational loop of the bicyclic peptide which would potentially interfere with the interaction of the peptide with its target molecule. MSETC was therefore selected for continued development as it appeared to provide a relatively stable/equal two looped structure with a short tail. In contrast, the cysteine residues of the QIMRB-3 construct were particularly close together (creating a very short loop). Accordingly, MSETC was selected to take forward as the optimal bicyclic inhibitor based on this sequence and structure analysis (see, FIG. 4).

### Initial Activity Studies of the Bicyclic Peptide Inhibitors.

These data clearly show that the bicyclic inhibitor (FIG. 4A) dissociates the SETDB1:IMPα interaction as well as the ATF7IP:SETDB1 interaction. This protein structure model demonstrates how the bicyclic peptide inhibitor MSETC, blocks interaction between SETDB1 and IMPα1 or SETDB1 and ATF7IP. Protein gel analysis (FIG. 4B) demonstrates that MSETC is able to bind to IMPα1, thus demonstrating that MSETC is able to target the IMPα1 interaction with SETDB1. The direct epigenetic SETDB1 pathways that are impacted by MSETC inhibition were also examined. In this regard, transcription analysis reveals that pathways involved with antigen presentation, viral mimicry, T-cell cytotoxicity, and the interferon-β pathway were significantly effected. These data clearly demonstrate that the bicyclic MSETC peptide is capable of inducing a re-invigoration of anti-tumour immune responses (FIG. 4C).

Analysis of proliferation reveals that the MSETC bicyclic peptide is superior to the linear prototypic SETDB1 peptide, in terms of proliferation of the TNBC metastatic cell line MDA-MB-231 (FIG. 4B) and the brain cancer clone of MDA-MB-231 (FIG. 4D,E). These values are quantified in Table 5.

TABLE 5
IC50 VALUES FOR SETDB1 LINEAR
AND BICYCLIC PEPTIDES

| Cell Line | Inhibitor | IC50 |
|---|---|---|
| MDA | 047_1 | 12.36 |
| | MSETC | 4.852 |
| MDA brain | 047_1 | 7.465 |
| | MSETC | 3.563 |

### Interrogation of Alternative Molecular Backbone Structures.

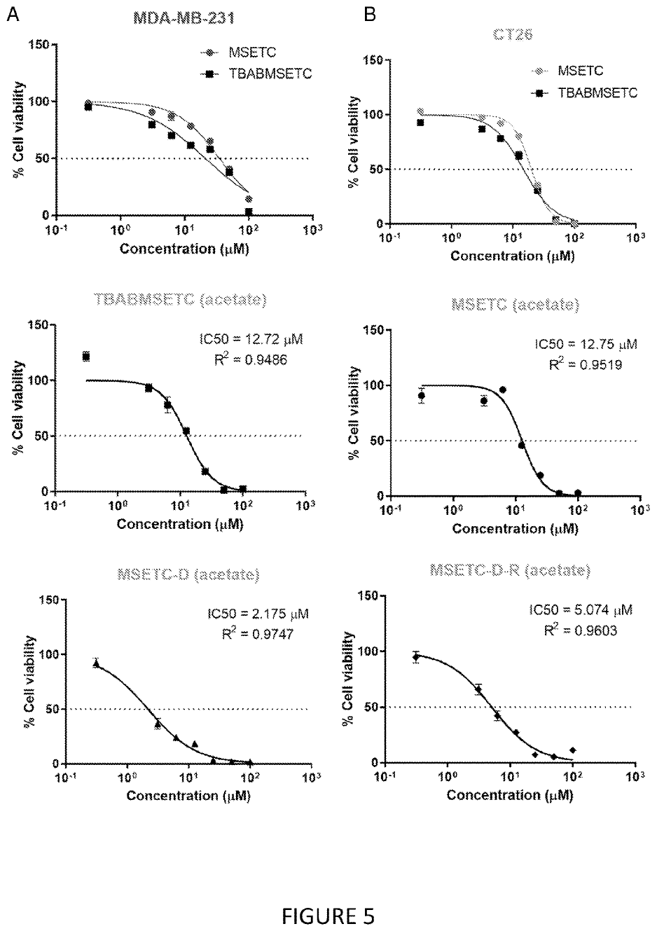

Analysis of proliferation reveals that the MSETC peptide on either bicyclic backbone (“scaffold”) was able to inhibit proliferation of all cancer cell lines with similar potency (FIG. 5A,B). Furthermore, the peptides MSETC-D or MSETC-D-R were significantly more potent at inhibiting LLC cell proliferation than either MSETC or TBAB-MSETC (FIG. 5 C-F and Table 6).

TABLE 6
MDA-MB-231

| Inhibitor | EC50 (uM) |
|---|---|
| MSETC | 35.04 |
| TBAB-MSETC | 21.20 |
| Inhibitor | EC50 (μM) |

CT26

| | |
|---|---|
| MSETC | 19.96 |
| TBAB-MSETC | 14.81 |

LLC1

| | |
|---|---|
| TBAB-MSETC | 12.72 |
| MSETC | 12.75 |
| MSETC-D | 2.175 |
| MSETC-D-R | 5.074 |

### Toxicity to Healthy Cells.

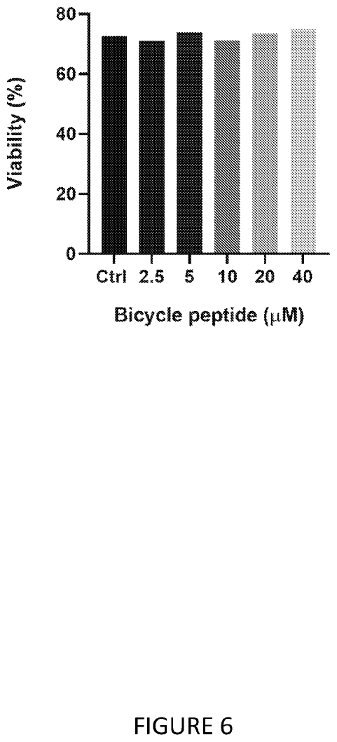

Analysis of impact on viability of PBMC derived from healthy donors reveals that the bicyclic inhibitor MSETC has no effect on healthy cell viability (FIG. 6).

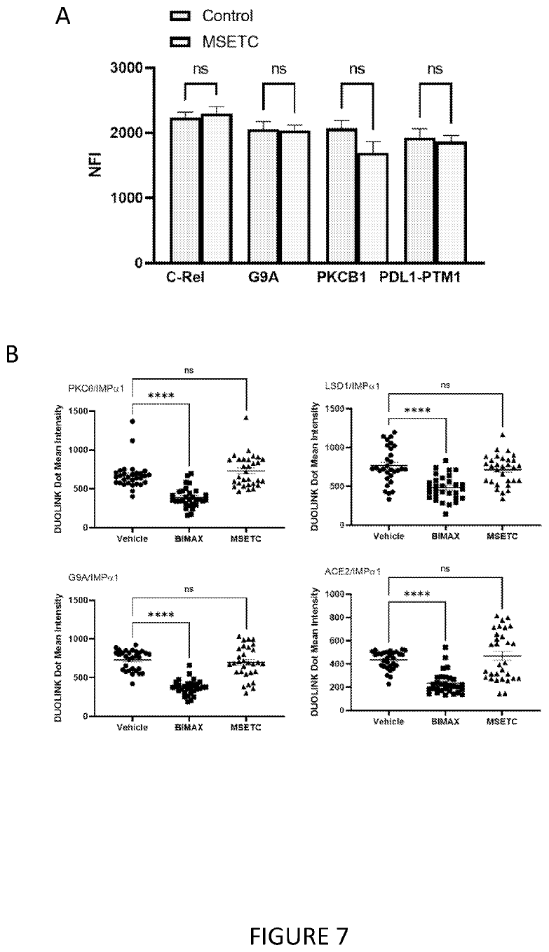

The inventors then sought to determine whether the bicyclic peptides had any clear off-target effects. MSETC had no off-target effect on any of the analysed nuclear proteins. DUOLINK analysis demonstrated that, while BIMAX (a general IMPα1 inhibitor) was able to significantly inhibit interaction with IMPα1 and four different IMPα1 targets (LSD1, G9A, PKC-θ, ACE2), the MSETC inhibitor did not inhibit these complexes demonstrating clear specificity for the nuclear shuttling of SETDB1 (FIG. 7). These data clearly demonstrate that MSETC is specific in its inhibition of the complex between SETDB1 and IMPα1, with no off-target effects observed.

### SETDB1 Localisation in Healthy Cells.

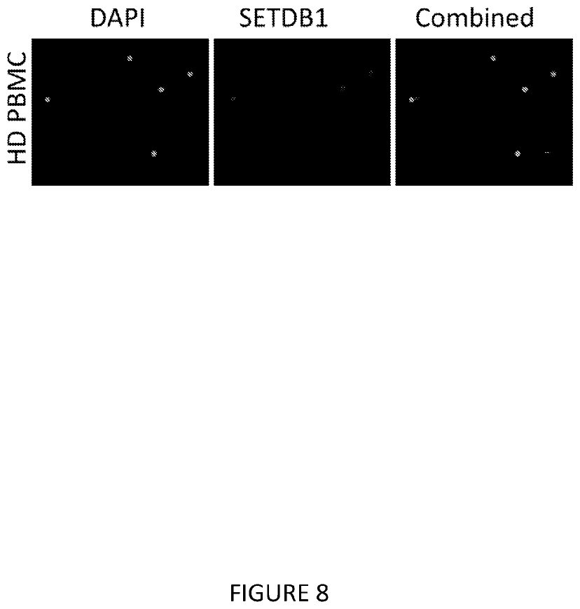

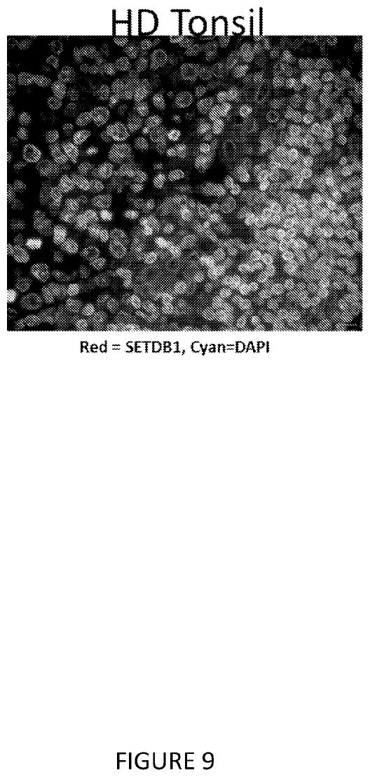

The present inventors then determined the cellular localization of SETDB1 in healthy cells. As clearly demonstrated in FIG. 8, in healthy PBMCs, SETDB1 was primarily localized to the cytoplasm (although at a low expression), with very little or no nuclear signal detected. A similar localisation pattern is observed for SETDB1 in healthy tonsil tissue (FIG. 9).

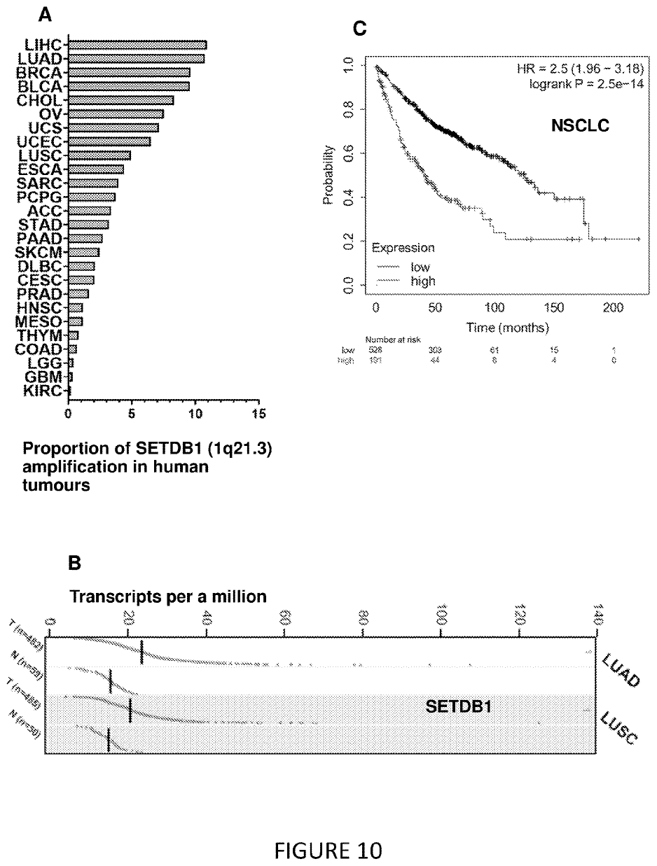

Analysis of publicly available data (TCGA) demonstrated that SETDB1 transcription is significantly enriched in a significant number of metastatic, invasive and therapeutically difficult to treat cancers (FIG. 10A). Additionally, SETDB1 is enriched in expression in cancer tissues compared to normal tissues in lung cancers (FIG. 10B) and a high expression of SETDB1 is linked to significantly worse survival for NSCLC patients (FIG. 10C).

### Materials & Methods

### Cancer Cell Viability Assays

Cell viability assays were performed as described in Example 1.

### PBMC Cell Viability Assay

PBMC cells were seeded at 5×10⁵ cells/well into a 96-well flat bottom tissue culture plate in a final volume of 100 μL. Cells were treated with different concentrations of peptides (200, 100, 50, 25, 12.5 and 6.25 μM) for 10 hours at 37° C. and 5% CO₂. Following inhibition, 10 μL of undiluted WST-1 cell proliferation reagent (Sigma-Aldrich, Cat #11644807001) was directly added into each well and the plate was mixed using a microplate spectrophotometer for 30 seconds (low speed) before returning the plate to the incubator. Absorbance was recorded at 450 nm after 1 hour using a microplate spectrophotometer (with 30 sec mix time). Data represent an independent experiment performed in triplicate, results are graphed as mean+/−standard error (SE).

### IFA Microscopy.

To examine the signature of C-REL, G9A, PKCb1 and our custom antibody PDL1-PTM1, in MDA-MB-231 TNBC cells, cells were permeabilised by incubating with 0.5% Triton X-100 for 15 min, blocked with 1% BSA in PBS and were probed with either C-REL, G9A, PKCb1 and our custom antibody PDL1-PTM1 visualized with a donkey anti-rabbit AF 488, anti-mouse AF 568, donkey anti-goat 647. Cover slips were mounted on glass microscope slides with ProLong Glass Antifade reagent (Life Technologies). Protein targets were localised by digital pathology laser scanning microscopy. Single 0.5 μm sections were obtained using an ASI Digital pathology microscope using 100× oil immersion lens running ASI software. The final image was obtained by averaging four sequential images of the same section. Digital images were analyzed using automated ASI software (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. Digital images were also analysed using ImageJ software (ImageJ, NIH, Bethesda, MD, USA) to determine the total cell fluorescence or cell surface only fluorescence for non-permeabilised cells. Digital images were analysed using ImageJ software (ImageJ, NIH, Bethesda, MD, USA) to determine the either the Total Nuclear Fluorescent Intensity (TNFI), the Total Cytoplasmic Fluorescent Intensity (TCFI). ImageJ software with automatic thresholding and manual selection of regions of interest (ROIs) specific for cell nuclei was used to calculate the Pearson's co-efficient correlation (PCC) for each pair of antibodies. PCC values range from: −1=inverse of co-localisation, 0=no co-localisation, +1=perfect co-localisation. The Mann-Whitney nonparametric test (GraphPad Prism, GraphPad Software, San Diego, CA) was used to determine significant differences between datasets.

Equivalent methods were used to examine the signature of SETDB1 in HD PBMCs.

### Cell Culture Methods

All breast cancer cell lines used were sourced from ATCC. MDA-MB-231 cell lines were maintained and cultured in DMEM (Invitrogen) supplemented with 10% FBS, 2 mM L-glutamine, and 1% PSN. MCF-7 cells were stimulated with 1.29 ng/mL phorbol 12-myristate 13-acetate (PMA) (Sigma-Aldrich) or 5 ng/mL recombinant TGF-β1 (R&D Systems) for 60 hours. For inhibitor studies, 2×10⁵ cells were seeded in 2 mL complete media in 6 well plates and incubated overnight at 37° C./5% CO₂. Cells were treated SETDB1 bicyclic peptides targeting the nuclear axis of SETDB1 or vehicle control. For microscopy studies, 4×10⁴ cells were seeded on coverslips and treated with inhibitors as described above. At each timepoint, coverslips were washed, fixed with 3.7% formaldehyde (Sigma) and stored at 4° C. until processing.

### Duolink Proximity Assay.

H1299 lung cancer cells were treated with MSETC (either MSETC or MSETC-D-isomer version (MSETC-D) or MSETC-D-isomer retroinverso (MSETC-D-R), IMPα1 inhibitor, BIMAX, or SETDB1 catalytic inhibitor, MTH. Cells were permeabilized and were probed with the DUOLINK ligation assay. Cover slips were mounted on glass microscope slides with Prolong nucleus glass Antifade reagent (Life Technologies). SETDB1 and IMPα1 DUOLINK or IMPα1 and four different IMPα1 targets (LSD1, G9A, PKC-0, ACE2 Digital images were analyzed using ImageJ software (ImageJ, NIH, Bethesda, MD, USA) and graphs represent the mean DOT Fluorescent Intensity in the nucleus or the cytoplasm compartments with significant differences calculated as per Kruskal-Wallis one-way ANOVA.

### Healthy Donor PBMC Isolation.

Whole blood was stored in EDTA tubes for circulating tumour cell identification. Unwanted cells were targeted for using glycophorin A on red blood cells (RBCs). Unwanted cells were then removed via centrifugation over a buoyant density medium Lymphoprep™ (Catalogue #07801). The purified PBMCs were then extracted as a highly enriched population from the interface between the plasma and the buoyant density medium and were harvested in 20% FBS in PBS.

### Opal Tissue Microscopy

To examine the signature of nuclear SETDB1 in healthy tonsil tissue, the OPAL staining kit for automatic staining using the automatic bondrx platform was used, in accordance with the manufacturer's instructions. Proteins were then mounted and localised as described in the IFA microscopy methods, above.

### Example 3

### SETDB1 Bicyclic Peptide Inhibitors in Cancer Models

### SETDB1 in Circulating Tumour Cells (CTCs).

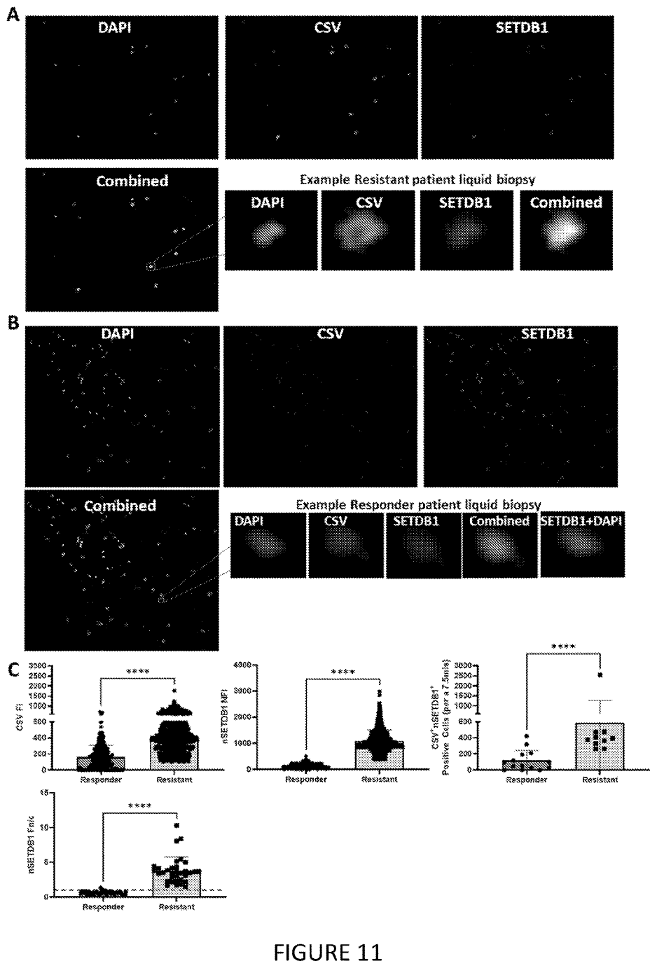

To examine the dynamics of SETDB1 in melanoma CTCs isolated from liquid biopsies, the present inventors divided samples based on response to immunotherapy. Samples were assessed as being from donors that were either immunotherapy “resistant” (FIG. 11A) or “responders” (FIG. 11B, as determined via iRECIST v1.1).

In this experiment, liquid biopsies were taken from 24 metastatic cancer patients undergoing immunotherapy. The metastatic cancer indications included melanoma, HNSCC, NSCLC, Merkel Cell, prostate cancer and renal cell cancer. Liquid biopsy samples were probed with a panel targeting mesenchymal CTCs (CSV positive).

Strikingly, SETDB1 clearly demonstrated a definite and significantly higher nuclear bias in the resistant cohort as compared to the responder cohort (FIGS. 11A, B). Additionally, all cells were also positive for cell surface vimentin, which is a marker for mesenchymal CTCs.

Overall the patient samples all displayed a positive SETDB1 signal and significantly increased nuclear SETDB1 signal was seen in the immunotherapy-resistant cohort (FIG. 11C). This also indicates that an increased cytoplasmic bias and decreased nuclear bias for SETDB1 is actually correlative with a favourable or complete response to immunotherapy.

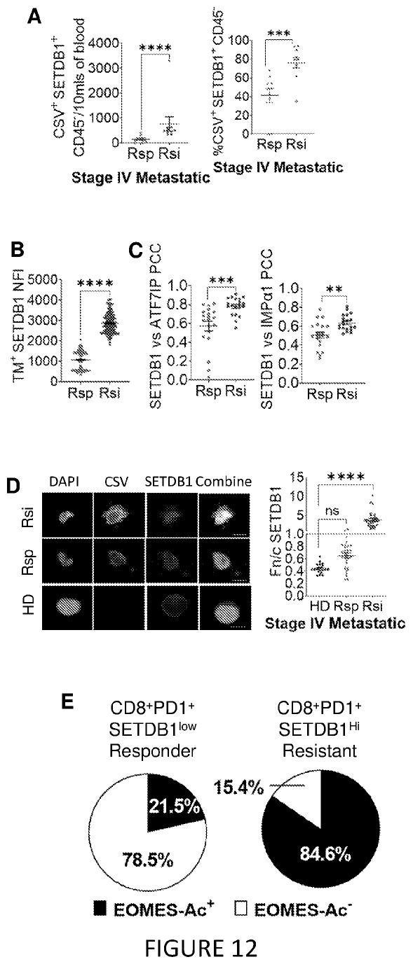

Increased total numbers of CTCs positive for SETDB1 and CSV were also observed (FIG. 12A), as well as an increased % population of cells positive for SETDB1 and CSV (FIG. 12A) in liquid biopsies of Stage IV metastatic cancer patients that are resistant to immunotherapy. Additionally, in metastatic tissues from resistant patients, nuclear expression of SETDB1 was similarly enriched (FIG. 12B). SETDB1 strongly co-localized with its protein interaction partner, ATF7IP, which stabilizes its nuclear expression and enhances its nuclear activity (FIG. 12C). This co-localization was strongly induced and more significant in Resistant patient liquid biopsies compared to Responder patient liquid biopsies (FIG. 12C). Analysis of the ratio of nuclear to cytoplasmic staining of SETDB1 also demonstrated (FIG. 12D) that SETDB1 had significantly increased nuclear bias in CTCs from patient liquid biopsies of metastatic cancers with resistant to immunotherapy.

SETDB1 also strongly co-localized with its nuclear shuttling interaction partner, IMPα1, which induces nuclear translocation (FIG. 12C). This co-localization was strongly induced and more significant in Resistant patient liquid biopsies compared to Responder patient liquid biopsies.

Overall the patient samples all displayed a positive SETDB1 signal and significantly increased nuclear SETDB1 signal was seen in the Resistant cohort. This also indicates that an increased cytoplasmic bias and decreased nuclear bias for SETDB1 is actually correlative with a complete response to immunotherapy.

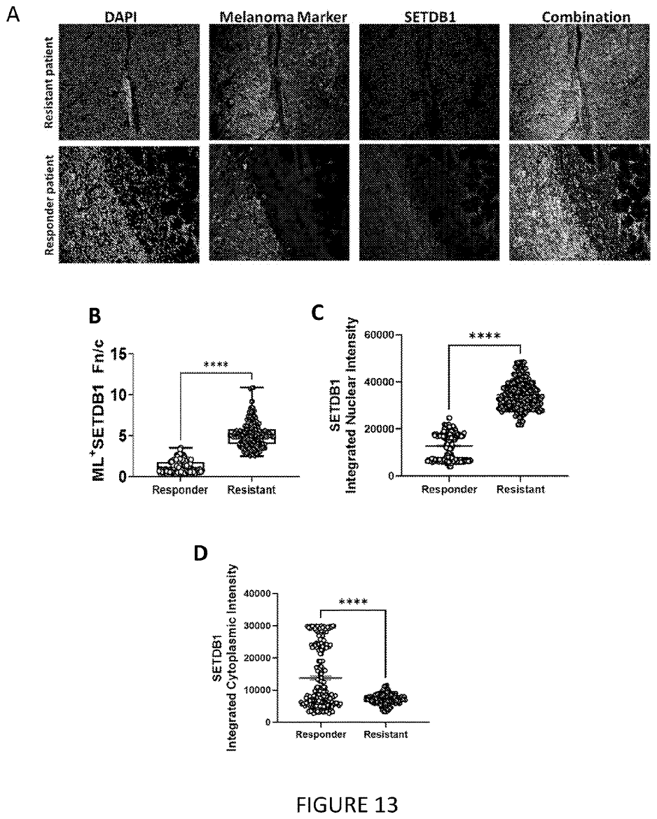

To investigate the cellular localization further, tissue IFA analysis was performed using the digital pathology platform describe herein. These data revealed that in tissue sections the resistant patient cohort had a clear increase in the nuclear bias for SETDB1 as compared to responder patient tissue sections (FIG. 13).

### Materials & Methods

### CTC Enrichment

Whole blood from stage IV metastatic cancer patients was stored in EDTA tubes for circulating tumour cell identification. The RosetteSep™ Human CD45 Depletion Cocktail was used to enrich tumour cells (CTCs) from whole blood by depleting CD45+ cells. Unwanted cells were targeted for depletion with Tetrameric Antibody Complexes recognizing CD45, CD66b and glycophorin A on red blood cells (RBCs). Unwanted cells were then removed via centrifugation over a buoyant density medium Lymphoprep™ (Catalogue #07801). The purified epithelial tumour cells were then extracted as a highly enriched population from the interface between the plasma and the buoyant density medium and were harvested in 20% FBS in PBS.

### IFA Microscopy Automated Imaging

To examine the signature of SETDB1, CSV, in Liquid biopsies from Stage IV metastatic samples, cells (processed as above) were permeabilised by incubating with 0.5% Triton X-100 for 15 min, blocked with 1% BSA in PBS and were probed with either CSV, SETDB1 visualized with a donkey anti-rabbit AF 488, anti-mouse AF 568, donkey anti-goat 647. Cover slips were mounted on glass microscope slides with ProLong Glass Antifade reagent (Life Technologies). Protein targets were localised by digital pathology laser scanning microscopy. Single 0.5 μm sections were obtained using a ASI Digital pathology microscope using 100× oil immersion lens running ASI software. The final image was obtained by averaging four sequential images of the same section. Digital images were analyzed using automated ASI software (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. For automated imaging and counting of cell populations, protein targets were localized by digital pathology laser scanning microscopy. Single 0.5 μm sections were obtained using an ASI Digital pathology (ASI Digital pathology is characterization of both the fluorescent intensity as per normal immunofluorescent imaging as well as the ability to count the population of cells positive or negative for antibodies, allow population dynamics to be investigation using powerful custom designed algorithms and automated stage. This also allows the imaging and counting of large cell numbers for statistical power) microscope using a 100× oil immersion lens running ASI software. The final image was obtained by averaging four sequential images of the same section. Digital images were analyzed using automated ASI software as described previously (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. Digital images were also analyzed using ImageJ software (ImageJ, NIH, Bethesda, MD, USA) to determine the total cell fluorescence or cell surface only fluorescence for non-permeabilized cells.

### Opal Tissue Microscopy

Responder or resistant to immunotherapy patient FFPE samples from stage IV melanoma patients were used to examine the signature of nuclear SETDB1, and melanoma cancer marker [HMB45+M2-7C10+M2-9E3](cancer cell marker) the OPAL staining kit for automatic staining use the automatic bondrx platform was used following them manufactures directions. Proteins were then mounted and localised as described in IFA microscopy. For automated imaging and counting of cell populations, protein targets were localized by digital pathology laser scanning microscopy. Single 0.5 μm sections were obtained using an ASI Digital pathology microscope using a 100× oil immersion lens running ASI software (ASI Digital pathology is characterization of both the fluorescent intensity as per normal immunofluorescent imaging as well as the ability to count the population of cells positive or negative for antibodies, allow population dynamics to be investigation using powerful custom designed algorithms and automated stage. This also allows the imaging and counting of large cell numbers for statistical power). The final image was obtained by averaging four sequential images of the same section. Digital images were analyzed using automated ASI software as described previously (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. Digital images were also analyzed using ImageJ software (ImageJ, NIH, Bethesda, MD, USA) to determine the total cell fluorescence or cell surface only fluorescence for non-permeabilized cells.

### Example 4

### Bicyclic Peptide Inhibitors Modify SETDB1 Cellular Localisation

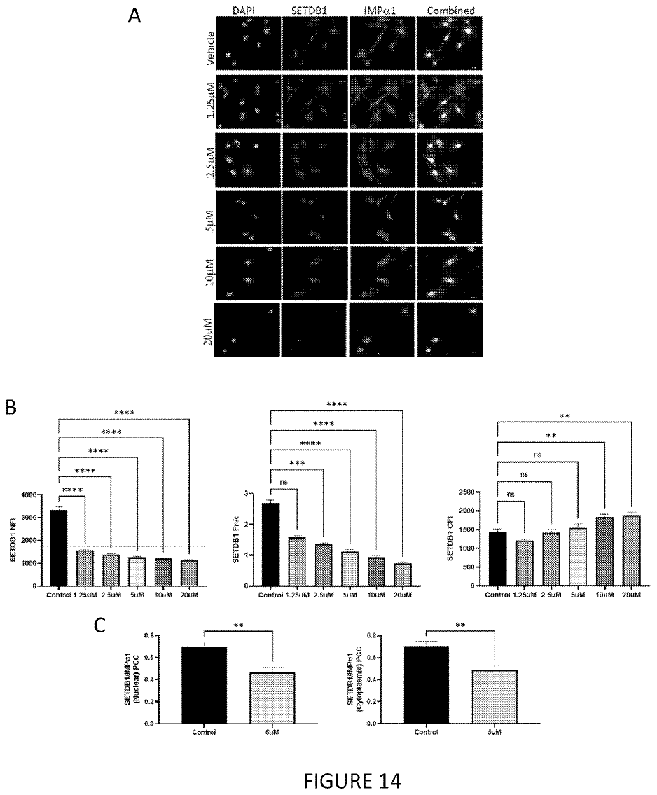

Treatment of cells with MSETC reduced the nuclear translocation of SETDB1 and the interaction (PCC) of SETDB1 and IMPθα1 within the cytoplasmic and nuclear compartments (FIG. 14A-C). The Fn/c of SETDB1 was also significantly reduced (FIG. 14B, middle panel). However, there was little to no effect on the cytoplasmic fraction of SETDB1 (FIG. 14B, right panel).

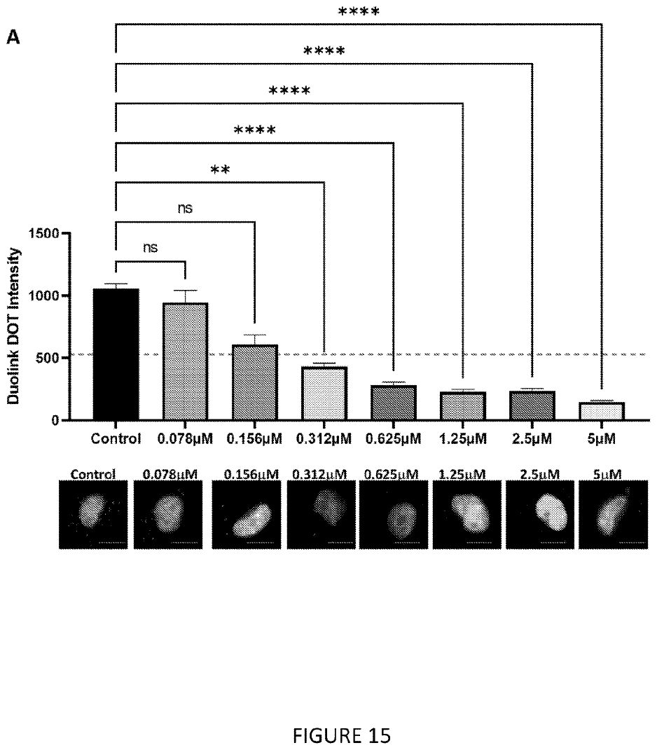

Next, the present inventors looked to determine how the concentration of the bicyclic peptide impacts on the interaction between SETDB1 and IMPα1. Treatment with MSETC reduced the nuclear translocation of SETDB1 and the interaction of SETDB1 and IMPα1 in a dose dependent manner (FIG. 15A). This reduction in interaction between SETDB1 and IMPα1 results in an inhibition of nuclear localization of SETDB1 (FIG. 15B).

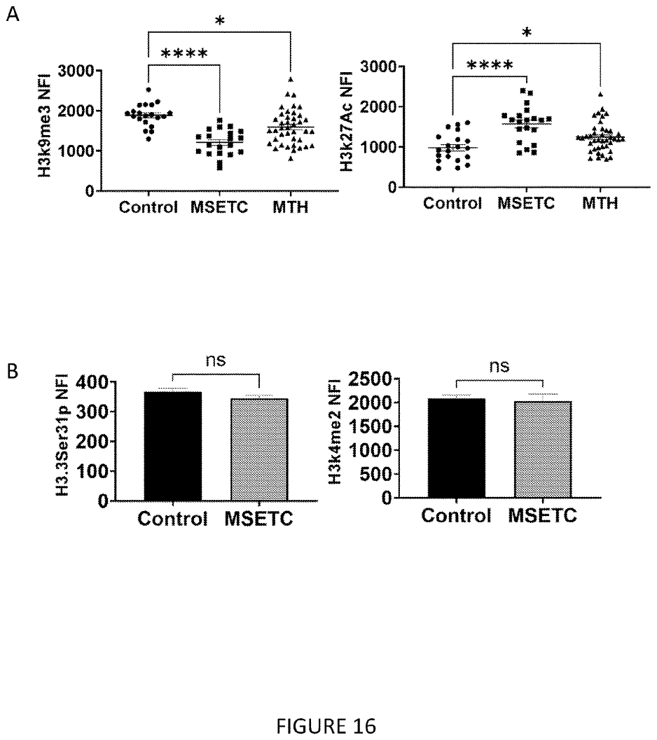

Treatment with the bicyclic peptide inhibitor MSETC reduced the nuclear activity and localization of SETDB1 and was superior to the catalytic SETDB1 inhibitor mithramycin A (FIG. 16A). This demonstrates the critical nature of targeting the nuclear axis over the targeting of the catalytic activity of SETDB1. MSETC had no effect on off-target histone markers H3.3Ser1p and H3k4me2 (FIG. 16B). These experiments were carried out to assess the impact of MSETC on the direct chromatin targets regulated by SETDB1, which demonstrated that MSETC is able to specifically inhibit the effects of SETDB1 on the chromatin template and associated histone marks. FIG. 16B demonstrates that histone marks not regulated by SETDB1 are not impacted by MSETC inhibition.

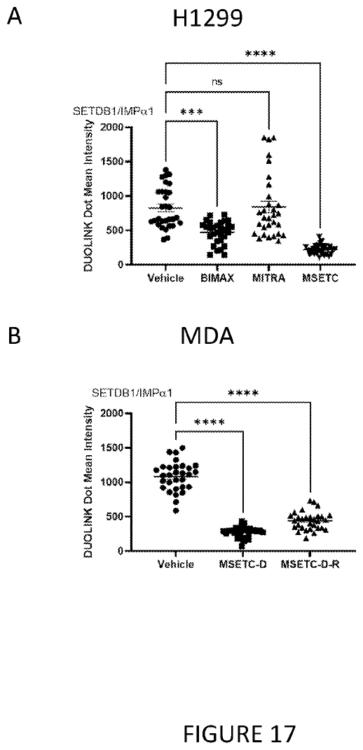

Next, to confirm if MSETC is superior to general inhibitors of either IMPα1 interaction (BIMAX; which impacts all IMPα1 interactions) or the catalytic activity of SETDB1 (MTH/MITRA) the inventors confirmed whether the novel bicyclic peptides are more inhibitory than general IMPα1 or SETDB1 inhibitors. These data demonstrate in the H1299 cancer cell line the bicyclic inhibitor MSETC exhibited far greater inhibition of the complexation between SETDB1 and IMPα1, as compared to both the general IMPα1 inhibitor BIMAX, and the SETDB1 catalytic inhibitor, MTH (FIG. 17A).

Upon validation of the MSETC bicyclic peptide as being superior to the general inhibitors, further optimization of the peptide was performed in an effort to increase the molecule half-life. Two additional bicyclic peptides were produced, a D-amino acid isomer for of MSETC (MSETC-D), which retained full sequence homology, and a D-amino acid isomer that was in a reteroinverso form of MSETC (MSETC-D-R). FIG. 17B shows that these two optimized forms are at least as potent as MSETC.

In addition, in silico analysis was used to create additional optimizations to reduce potential toxic side effects, improve hydrophilia and improve anti-tumour activity (see, Table 4, below). Increasing negative SVM score indicates reducing toxic potential and increasing hydrophilicity indicates more water soluble compound.

TABLE 7
Toxicity Screen

| | | SVM | | |
|---|---|---|---|---|
| MSETC-D | Sequence | Score | Hydrophilicity | Prediction |
| Prototype | GCKKRTKTWHCKGTLIAIQTVGPGCKKYKV | −0.43 | 0.15 | Non-Toxic |
| Sequence | | | | |
| Variant 1 | GCKKRTKTWHCKITLIAIQTVGPGCKKYKV | −0.76 | 0.09 | Non-Toxic |
| Variant 2 | GCKKRTKTWHCKGTLIAIQTVGPRCKKYKV | −0.72 | 0.25 | Non-Toxic |
| Variant 3 | GCKKRTKTWHCKITLIAIQTVGPRCKKYKV | −1.07 | 0.19 | Non-Toxic |
| Variant 4 | GCKKRTKTWHCKITLIAIQTVGPRCKKYRV | −1.28 | 0.19 | Non-Toxic |
| Variant 5 | GCKKRTKTWHCKITKIAIQTVGPRCKKYKV | −1.19 | 0.35 | Non-Toxic |
| Variant 6 | GCKKRTKTWHCKITKIAIQTVGPRCKKYRV | −1.39 | 0.33 | Non-Toxic |
| Variant 7 | ACKKRTKTWHCKITKIAIQTVGPRCKKYRV | −1.48 | 0.33 | Non-Toxic |
| Variant 8 | DCKKRTKTWHCKITKIAIQTVGPRCKKYRV | −1.44 | 0.45 | Non-Toxic |
| Variant 9 | GCKKRTKTWHCKITKIAIQTVGPRCKIYRV | −1.68 | 0.19 | Non-Toxic |

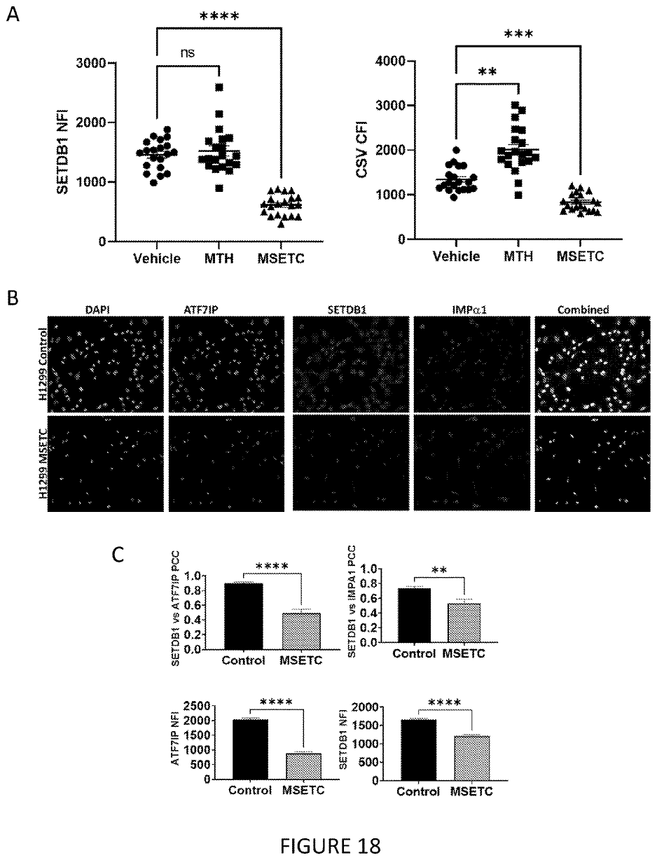

Treatment of the H1299 cell line, from non-small cell lung carcinoma, with MSETC reduced the nuclear expression of SETDB1, whereas MTH had no effect (FIG. 18A). MSETC was also able to abrogate expression of CSV whereas MTH induced an increase in expression of the mesenchymal metastatic marker CSV (FIG. 18A).

ATF7IP is a nuclear stabilization partner of SETDB1 and enhances its nuclear activity. Treatment with MSETC reduced the nuclear expression of SETDB1 as well as nuclear ATF7IP (FIG. 18B), and abrogated the PCC (co-localization) of SETDB1 with either ATF7IP or IMPα1 (FIG. 18C).

### Materials & Methods

### Duolink Proximity Assay.

MDA-MB-231 cells were treated with MSETC bicyclic peptide inhibitor; with concentrations ranging from 5 μM to 0.078 μM. MDA-MB-231 cells were treated with MSETC bicyclic inhibitor or control and were permeabilized and were probed with the DUOLINK ligation assay. Cover slips were mounted on glass microscope slides with Prolong nucleus glass Antifade reagent (Life Technologies). SETDB1 and IMPα1 DUOLINK digital images were analyzed using ImageJ software (ImageJ, NIH, Bethesda, MD, USA) and graphs represent the mean DOT Fluorescent Intensity in the nucleus or the cytoplasm compartments with significant differences calculated as per Kruskal-Wallis one-way ANOVA.

### IFA and Cell Culture Methods.

IFA microscopy and cell culture methods were performed as described in detail, above.

### Example 4

### SETDB1 Bicyclic Peptide Inhibitors—Gene Regulation

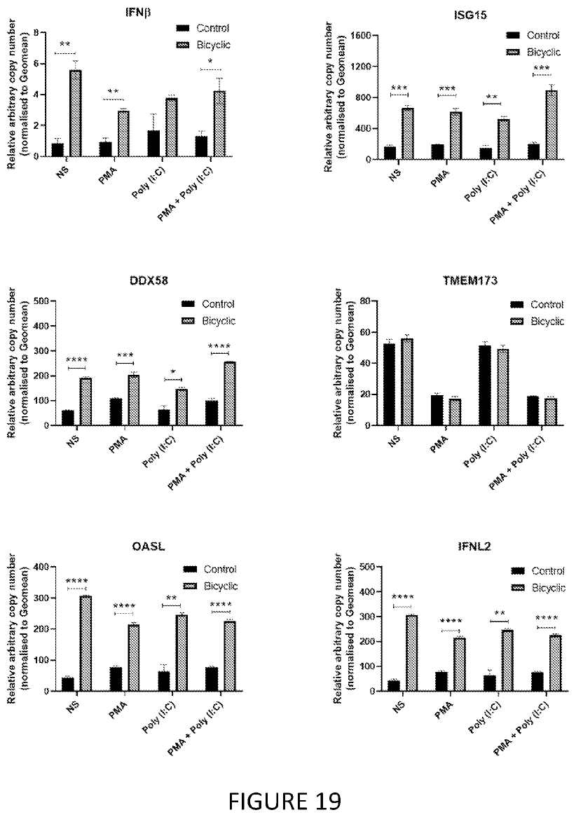

The MSETC SETDB1 bicyclic peptide induces increased expression of genes associated with the IFN signalling pathway, these genes include increased expression of IFNβ, IFNL2, DDX58, OASL, and ISG15 (FIG. 19). Interestingly increased gene expression occurs regardless of stimulation with PMA and/or Poly I:C. (epithelial versus mesenchymal) and the expression of TMEM173 (STING) remains unaltered with peptide treatment. A previous study (Wang, G., Long, J., Gao, Y. et al. Nat Cell Biol. 2019; 21, 214-225) has shown that SETDB1 methylation promotes cell surface AKT in tumorigenesis. Treatment with the MSETC bicyclic peptide significantly abrogates cell surface AKT determined by quantifying AKT expression in non-permeabilized cancer cells. Overall the effect of MSETC is not only to remove SETDB1 from its nuclear chromatin role but also to abrogate its cytoplasmic methylation of pro-tumour proteins.

The SETDB1 bicyclic peptide induces increased expression of a large cohort of genes associated with viral mimicry and immunogenicity/immune visibility signalling pathway (FIG. 19). Interestingly, increased gene expression occurs regardless of stimulation with PMA (epithelial versus mesenchymal) (FIG. 19). This indicates the inhibition by MSETC is able to induce immune visibility of the tumours to add in immune mediated killing of the tumour and metastatic sites.

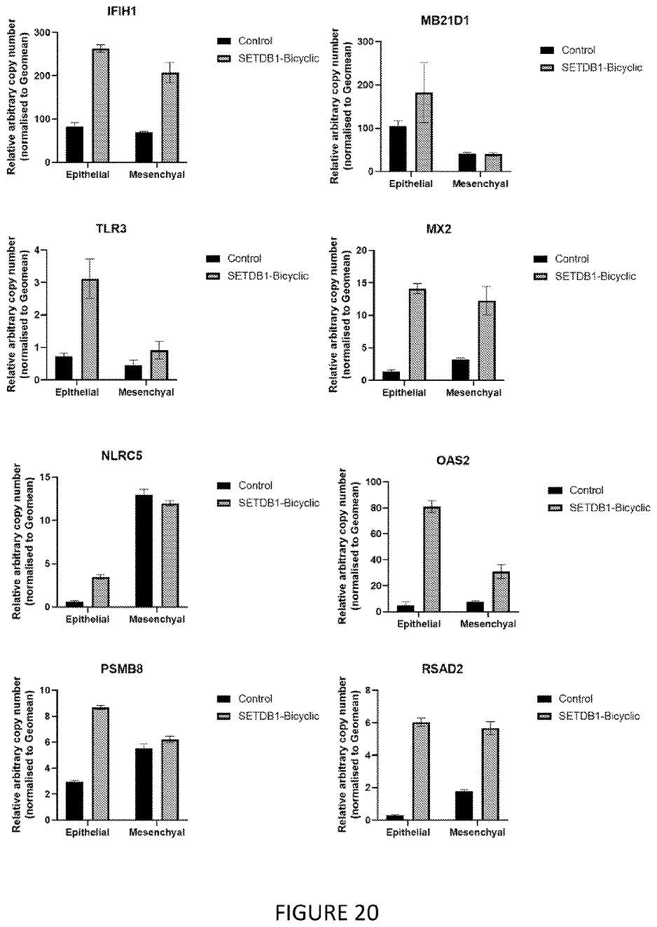

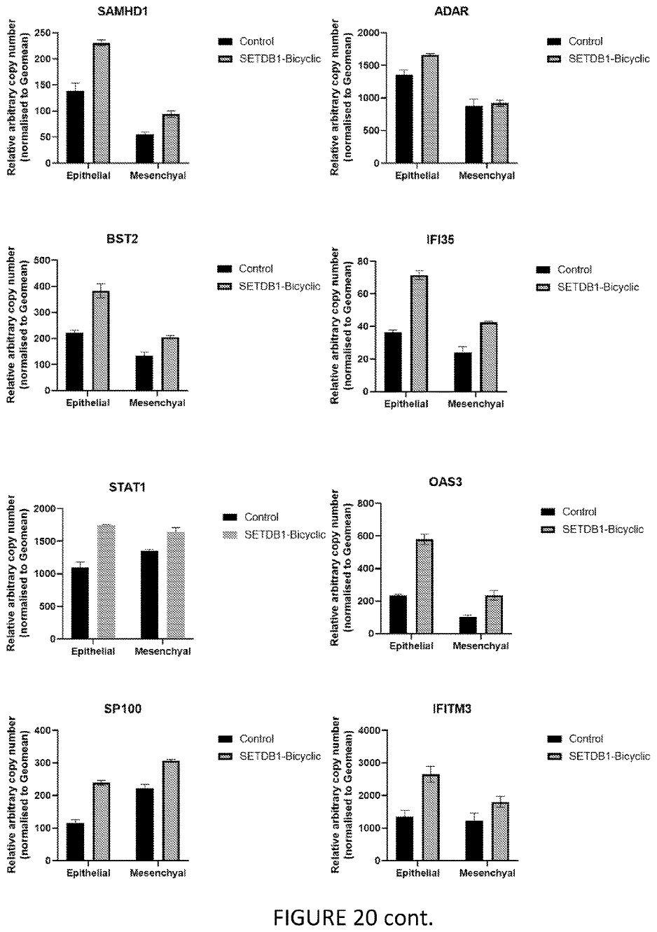

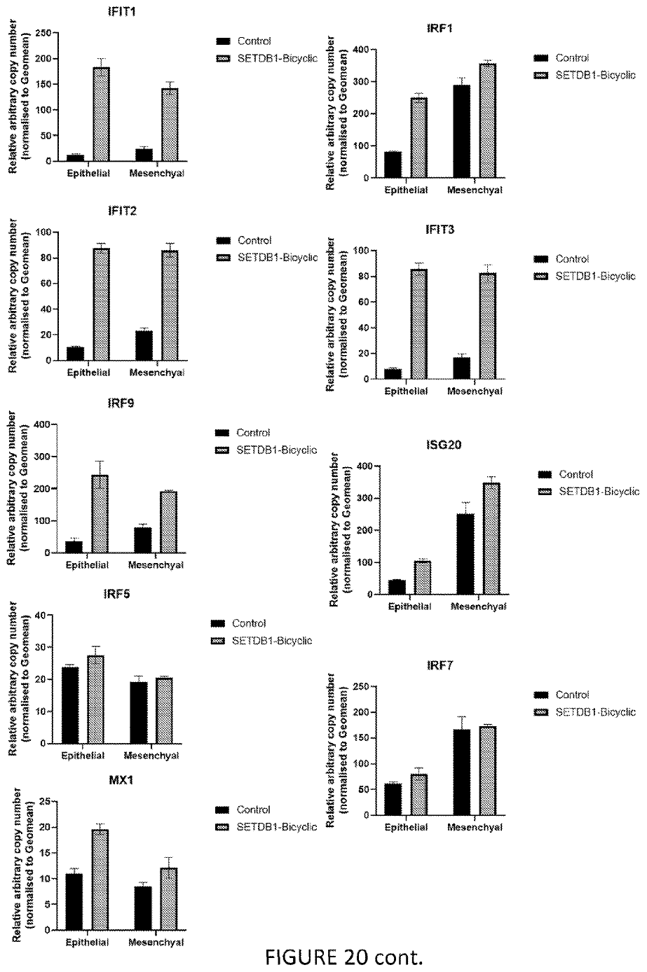

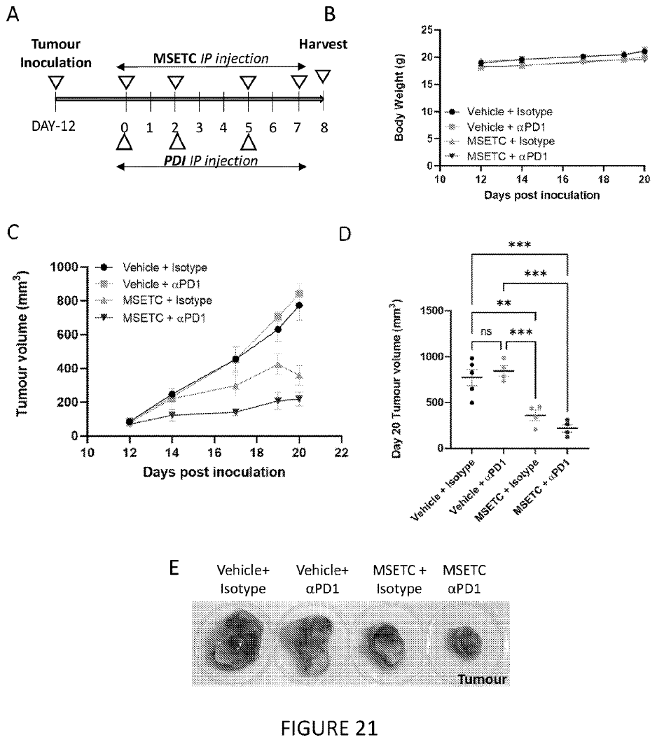

The inventors then carried out transcriptomic investigation the expression of genes associated with the immune visibility of the cancer cell. This was carried out to determine the ability of MSETC to induce immune mediated against cancer cells and inhibit the tumours ability to evade or hide from the immune system by down regulating various pathways associated with immune visibility. The present inventors found that the SETDB1 bicyclic peptide induces increased expression of a large cohort of genes associated with viral mimicry and immunogenicity/immune visibility signalling pathway (FIG. 20), Interestingly increased gene expression occurs regardless of stimulation with PMA (epithelial versus mesenchymal). This indicates the inhibition by MSETC is able to induce immune visibility of the tumours to add in immune mediated killing of the tumour and metastatic sites (FIGS. 19 to 26).

### Materials & Methods

### RNA Extraction and CDNA Synthesis

Cells were lysed in 500 μL of TRizol Reagent (Invitrogen) before RNA isolation using the Direct-zol RNA Miniprep Kit (Zymo, R2052) and treated with RNase-free DNase I (Qiagen, 79254) following the manufacturers protocol. The Nanodrop (Thermofisher) was then used to determine the RNA concentration and purity (A260/A280). RNA (1 g) was then reverse-transcribed to cDNA using the Superscript VILO IV Master Mix (Thermo Fisher, 11756050) following the suppliers protocol. CDNA was diluted 1:20 with RNase-free water for RT-qPCR.

### RT-qPCR

Gene transcription was analysed using Taqman Gene Expression Master Mix (Life Technologies, 4369016) on the ABI Viia 7 real-time PCR machine (Applied Biosystems). Cts were converted to arbitrary copy numbers and normalized to the geomean of the house-keeping genes ACTB and PP/A. The following human Taqman probes were used: Human: IFNB1, ISG15, DDX58, TMEM173, OASL, IFNL2, ACTB, GAPDH; Mouse: Ifna1, Ifnb1, Isg15, Oasl, Ddx58, Ifih1, Tlr3, Actb, Ubc. No template and no reverse-transcriptase controls were included to exclude genomic DNA contamination.

### Example 5

### Bicyclic Peptide (MSETC)+/−PD1 Therapy in In Vivo Cancer Model

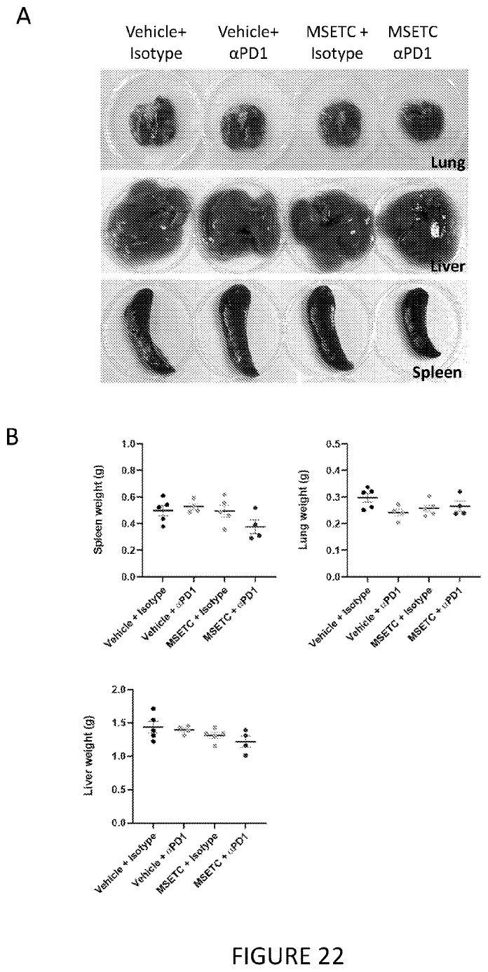

To further examine the therapeutic benefit of targeting nuclear SETDB1, the inventors used a TNBC (triple negative breast cancer) immunotherapy-resistant tumour mouse model. The 4T1 model represents a highly aggressive, metastatic tumour model that is difficult to treat. Therefore, this model is an excellent test to determine the anti-tum]mour capability of MSETC to abrogate and inhibit tumour burden and metastatic burden. In the 4T1 IO-resistant metastatic mouse model treatment with PD1 had no effect on tumour burden, whereas monotherapy MSETC or combination therapy had significant impact on tumour burden but no effect on mouse body weight (FIG. 22).

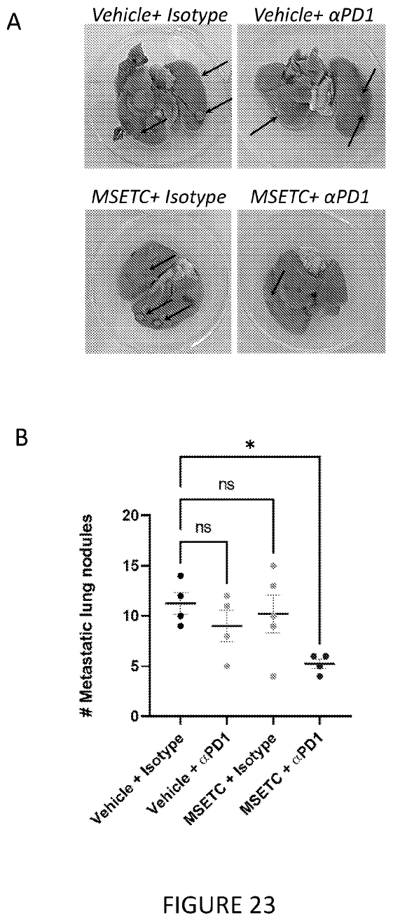

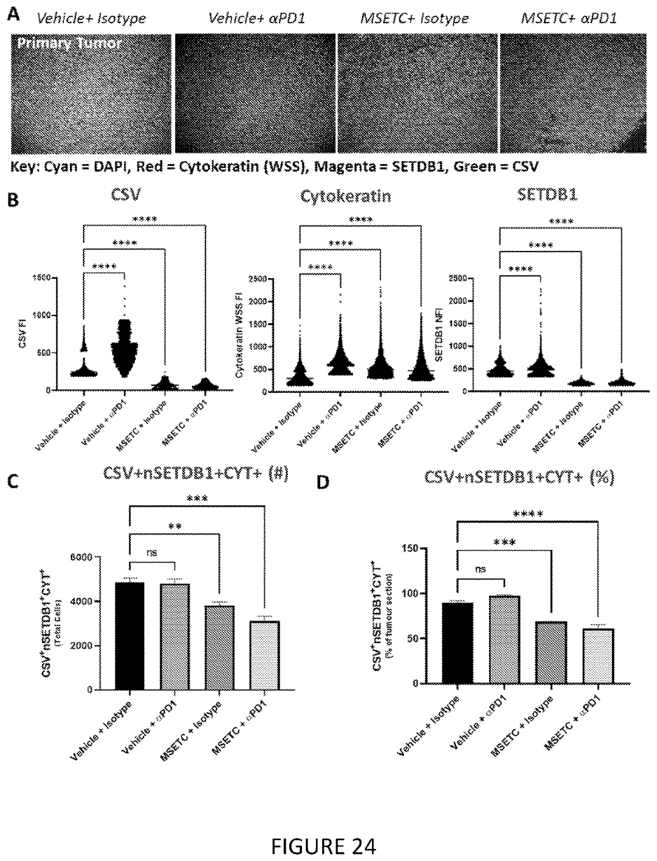

In the 4T1 IO-resistant metastatic mouse model treatment MSETC or combination therapy had significant impact on tumour burden but no effect on mouse organ (liver, spleen, lung) weight (FIG. 23). It was also found the PD1 had no effect on metastatic burden, whereas MSETC combination therapy had significant impact on metastatic lung burden (see, FIG. 24).

The 4T1 mouse tumour burden model demonstrated that treatment with MSETC, both monotherapy and in combination with αPD1, reduces tumour volume in the 4T1 model of metastatic breast cancer. MSETC treatment does not alter murine body weight. Furthermore, even without αPD1 combination therapy, treatment with MSETC reduces overall tumour weight. MSETC treatment does not alter lung, liver or spleen weights. Finally, MSETC and αPD1 combination therapy reduces lung metastasis. This indicates that in the highly aggressive mouse model for TNBC, MSETC is capable as a monotherapy of abrogating tumour burden as well as in this IO-resistant model inducing immunotherapy responses. These data suggest that in both metastatic and IO-resistant human cancers (such as TNBC), MSETC has a clear therapeutic effect effective for treating metastatic, IO-resistant cancers. Furthermore, the bicyclic peptide inhibitor has been shown to have no toxic side effects.

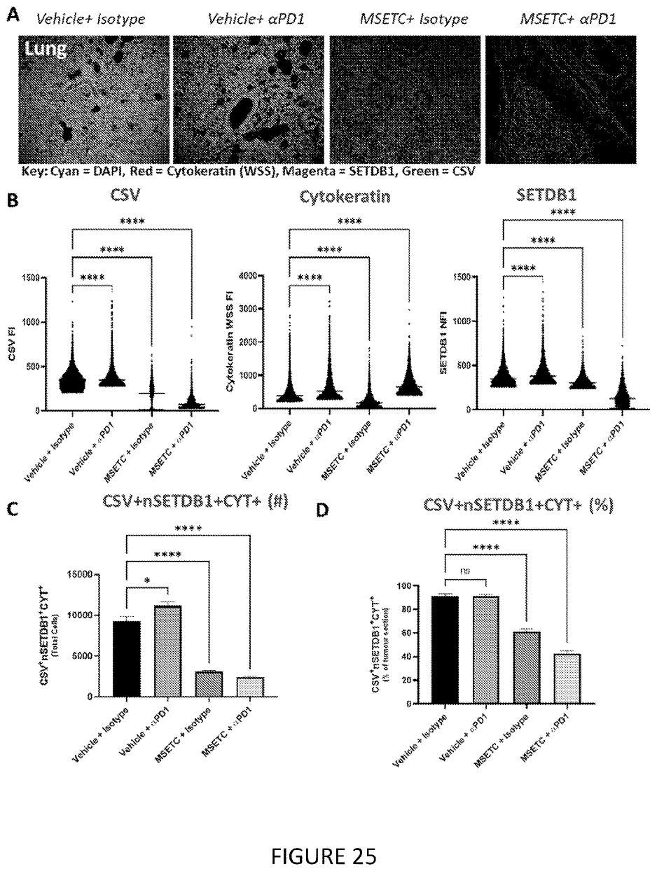

In the 4T1 IO-resistant metastatic mouse model primary tumour treatment with PD1 enhanced expression of mesenchymal marker CSV and nuclear SETDB1 but had no effect on overall cell numbers (see, FIG. 25). Whereas monotherapy MSETC or combination therapy had significant inhibition of CSV, SETDB1 expression and significantly reduced overall positive cells for those markers as well.

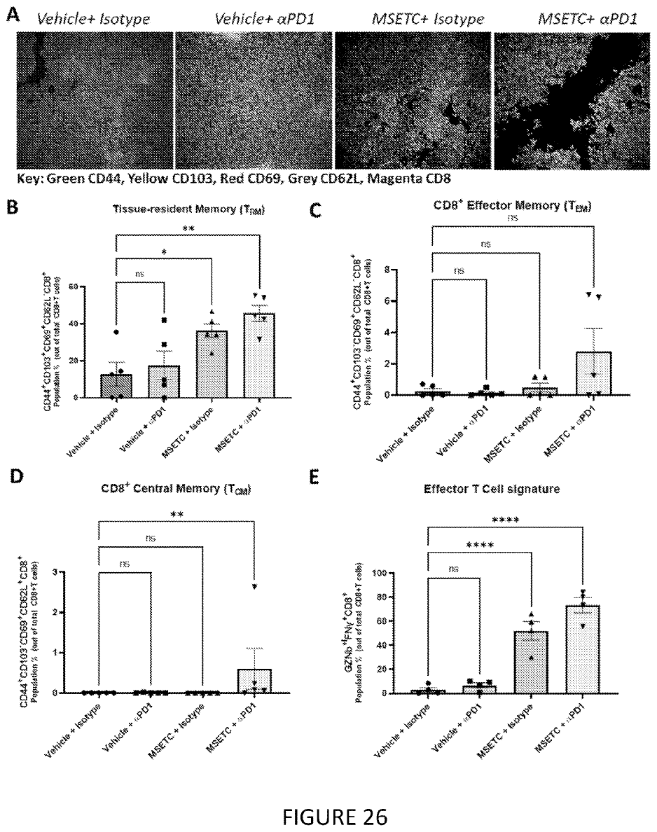

Similarly, in the 4T1 IO-resistant metastatic mouse model lung cancer lesions treatment with PD1 enhanced expression of mesenchymal marker CSV and nuclear SETDB1 but had no effect on overall cell numbers (see, FIG. 26). Whereas monotherapy MSETC or combination therapy had significant inhibition of CSV, SETDB1 expression and significantly reduced overall positive cells for those markers as well.

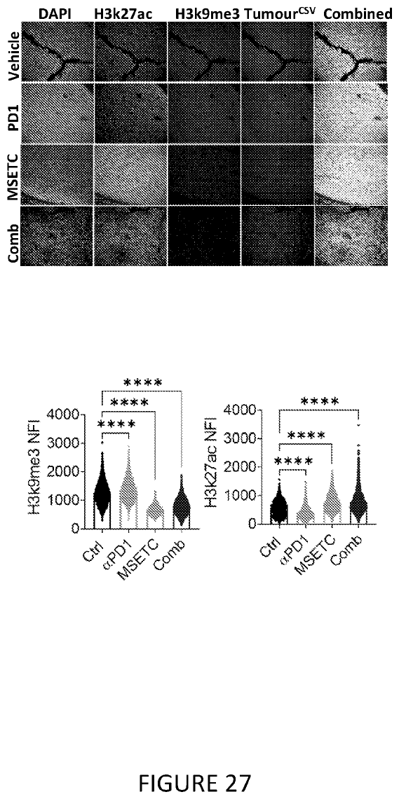

The inventors also demonstrated in the in vivo mouse models that MSETC is capable of hitting the nuclear activity of SETDB1 as demonstrated by inhibiting the SETDB1 histone mediated marker H3k9me3, as well as the active mark H3k27ac which is induced following inhibition of H3k9me3 (see, FIG. 27).

### Materials & Methods

### Animal Studies

Six-week-old female Balb/c mice were purchased from the Animal Resources Centre (ARC) and allowed to acclimatize for 1 week prior to use. All experimental procedures were performed in accordance with the guidelines and regulations approved by the QIMR Berghofer Animal Ethics committee. Mice were shaved at the site of inoculation prior to subcutaneous injection with 1×10⁵ 4T1 cells in 100 μL PBS into the right mammary gland. Treatments were started when tumours reached approximately 80 mm³. Tumours were measured using external calipers and volumes calculated using a modified ellipsoidal formula ½ (a/b2), where a=longest side and b=shortest side. For combination therapy experiments, mice were treated with vehicle (saline) or MSETC (20 mg/kg) three times/week in combination with anti-PD1 or isotype control (10 mg/kg) twice weekly by intraperitoneal injection. Mice were monitored daily, and tumour volumes and body weights were measured thrice weekly. Once tumours reached the maximal limit (1000 mm³) in the vehicle group, all tumours and associated metastatic organs (lung, liver, spleen) were harvested, imaged, weighed and sectioned for associated analysis.

### Assessment of Metastatic Lung Nodules

Excised lungs were fixed with Bouin's solution overnight, washed extensively in PBS and stored in 70% ethanol. Metastatic lung nodules were observed and counted under magnifying glass.

### Example 6

### MSETC-D-R Shows Enhanced Efficacy in Murine TNBC Model

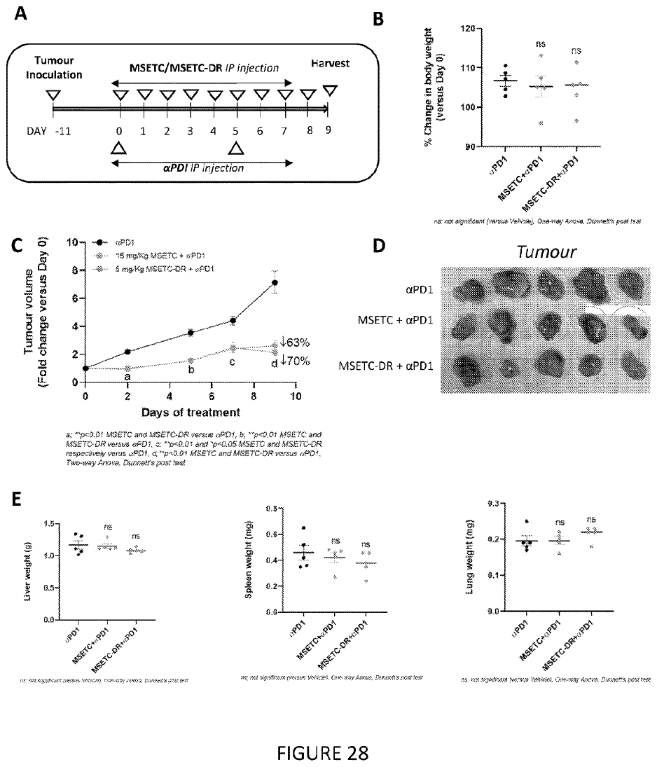

To compare the efficacy of MSETC and MSETC-D-R, we used the highly aggressive 4T1 murine model of TNBC and an immunotherapy-resistant model. Once primary tumours were established, Balb/c mice underwent daily treatment with MSETC (15 mg/kg) or MSETC-D-R (5 mg/kg) by intraperitoneal injection in combination with anti-PD1 immunotherapy (FIG. 28A). Importantly, peptide treatment did not induce toxicity in mice for either peptide, as body weights did not decline over the treatment period (FIG. 28B). MSETC treatment at 15 mg/kg in combination with anti-PD1 resulted in a 63% reduction in primary tumour volume after 9 days of treatment (FIG. 28C,D). Notably, MSETC-D-R had a 70% decrease using one-third of the dose, at 5 mg/kg (FIG. 28C,D). In support of our body weight data, examination of the major organs including the liver, spleen and lung revealed no significant changes in organ weight following treatment with MSETC or MSETC-D-R (FIG. 28E). MSETC-D-R shows greater efficacy compared to MSETC due to the retro-reverso form of MSETC-DR being less prone to cleavage by proteases and hence a more stable configuration.

### Example 7

### MSETC and MSETC-D-R Demonstrate Reduction in Cell Migration

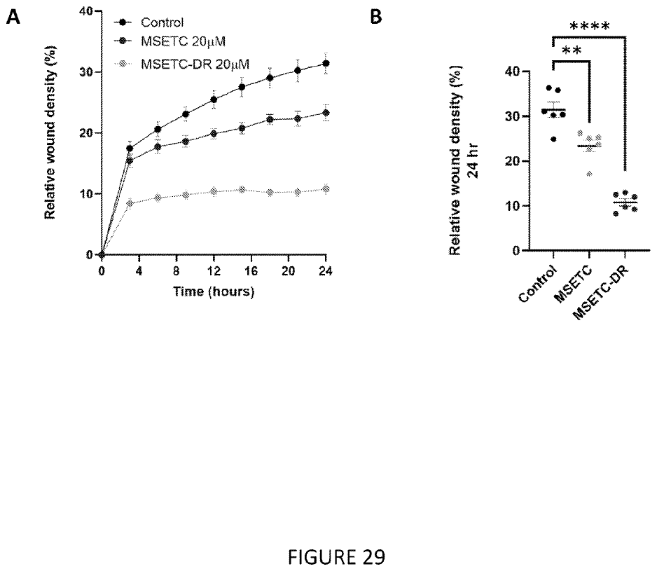

To determine the effect of MSETC and MSETC-D-R on cancer cell invasion, the inventors performed a wound healing assay using the human epithelial MCF-7 breast cancer cell line. Firstly, MCF-7 cells were treated with phorbol 12-myristate 13-acetate (PMA) and transforming growth factor-β (TGF-β) prior to the initiation of the scratch on the cell monolayer in order to induce a mesenchymal invasive phenotype. Cells were then treated with MSETC or MSETC-D-R and images captured at regular intervals over a 24 hour period (FIG. 29A). MSETC treatment significantly reduced cancer cell migration by 29% after 24 hours (FIG. 29B). Importantly, MSETC-D-R demonstrated an even greater reduction in cell migration, with a 68% reduction. These data suggest that the MSETC-D-R displays greater efficacy than MSETC at inhibiting cancer cell invasion.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A method of preventing or reducing nuclear localisation of a SETDB1 polypeptide, the method comprising contacting the SETDB1 polypeptide and/or an IMPα polypeptide to an agent that inhibits binding of the SETDB1 polypeptide to the IMPα polypeptide.

2. A method of preventing or reducing SETDB1 polypeptide nuclear localisation in a cancer cell, the method comprising contacting the cell with an agent that inhibits binding of the SETDB1 polypeptide to an IMPα polypeptide.

3. A method of treating or preventing a cancer in a subject, the method comprising administering to the subject an agent that inhibits binding of a SETDB1 polypeptide to an IMPα polypeptide.

4. The method of claim 3, wherein the cancer is associated with at least some SETDB1 polypeptide being present in the cell nucleus.

5. The method of any one of claims 2 to 4, wherein the cancer is selected from breast, prostate, lung, bladder, pancreatic, colon, liver, or brain cancer, or melanoma, or retinoblastoma.

6. The method of any one of claims 1 to 5, wherein the agent inhibits the binding of the SETDB1 polypeptide to the IMPα polypeptide, but does not inhibit the binding of any other polypeptide to the IMPα polypeptide.

7. The method of any one of claims 1 to 6, wherein the agent directly binds to the IMPα, polypeptide.

8. The method of any one of claims 1 to 7, wherein the agent reduces the amount of SETDB1 polypeptide that is present in the cell nucleus.

9. The method of any one of claims 1 to 8, wherein the agent reduces the ratio of nuclear SETDB1 polypeptide to cytoplasmic SETDB1 polypeptide.

10. The method of any one of claims 1 to 9, wherein the agent is a proteinaceous molecule comprising, consisting, or consisting essentially of an amino acid sequence corresponding to the nuclear localisation sequence (NLS) of a SETDB1 polypeptide.

11. The method of claim 10, wherein the proteinaceous molecule comprises, consists, or consists essentially of the amino acid sequence set forth in any of SEQ ID NOs: 1-6, or a at least 85% sequence identity to the sequence set forth in any one of SEQ ID NOs: 1-6.

12. The method of claim 10 or claim 11, wherein the proteinaceous molecule comprises, consists, or consists essentially of the amino acid sequence set forth in SEQ ID NO: 2 or SEQ ID NO: 6.

13. The method according to any one of claims 2-12, further comprising administering one or more further cancer therapies.

14. The method according to claim 13, wherein the further cancer therapy is a chemotherapeutic agent.

15. The method of claim 13, wherein the further cancer therapy is an immunotherapy.

16. The method of claim 15, wherein the immunotherapy is an immune checkpoint inhibitor.

17. The method according to any one of claims 1-16, wherein the proteinaceous molecule comprises, consists or consists essentially of an amino acid sequence corresponding to residues 206 to 232 of the full length human SETDB1 protein.

18. The method according to any one of claims 1 to 17, wherein the proteinaceous molecule is a fragment of a nuclear localisation sequence (NLS).

19. The method according to claim 17 or claim 18, wherein the proteinaceous molecule comprises 50 amino acid residues or less.

20. The method according to claim 19, wherein the proteinaceous molecule is distinguished from SETDB1 by the addition, deletion and/or substitution of at least one (e.g., 1, 2, 3, 4, 5, 6, 7, 8, 9 etc.) amino acid corresponding to residues 206 to 232 of the full length human SETDB1 protein.

21. A method of producing a proteinaceous molecule that inhibits or reduces nuclear localization of a SETDB1 polypeptide, the method comprising:
a) contacting a cell with a proteinaceous molecule comprising, consisting or consisting essentially of an amino acid sequence corresponding to residues 206 to 232 of the full length human SETDB1 protein; and
b) detecting a reduction in or inhibition of the nuclear localization of the SETDB1 polypeptide in the cell relative to a normal or reference level of nuclear localization in the absence of the proteinaceous molecule.

22. The method according to claim 21, wherein the proteinaceous molecule is distinguished from SETDB1 by the addition, deletion and/or substitution of at least one (e.g., 1, 2, 3, 4, 5, 6, 7, 8, 9 etc.) amino acid corresponding to residues 206 to 232 of SETDB1.

23. An isolated or purified proteinaceous molecule represented by Formula I:
GKKRX₁KX₂WHX₃GTLIX₄IQTVGPGKKX₅KVK
wherein:
Z₁ and Z₂ are independently absent or are independently selected from at least one of a proteinaceous moiety comprising from about 1 to about 50 amino acid residues (and all integer residues in between), and a protecting moiety;
X₁ is selected from Thr, Arg, and modified forms thereof;
X₂ is selected from Thr, Leu, and modified forms thereof;
X₃ is selected from Lys, Gly, and modified forms thereof;
X₄ is selected from Ala, Pro, and modified forms thereof; and
X₅ is selected from Tyr, Lys, and modified forms thereof.

24. The proteinaceous molecule according to claim 23, wherein Z₁ is absent.

25. The proteinaceous molecule according to claim 23 or 24, wherein Z₂ is absent.

26. The proteinaceous molecule of any one of claims 23 to 25, wherein X₁ is Thr.

27. The proteinaceous molecule of any one of claims 23 to 26, wherein X₂ is Thr.

28. The proteinaceous molecule of any one of claims 19 to 23, wherein X₃ is Lys.

29. The proteinaceous molecule of any one of claims 19 to 24, wherein X₄ is Ala.

30. The proteinaceous molecule of any one of claims 19 to 25, wherein X₅ is Tyr.

31. The proteinaceous molecule of any one of claims 19 to 21, wherein X₁ is Arg.

32. The proteinaceous molecule of claim 27, wherein X₂ is Leu.

33. The proteinaceous molecule of claim 27 or claim 28, wherein X₃ is Gly.

34. The proteinaceous molecule of any one of claims 27 to 29, wherein X₄ is Pro.

35. The proteinaceous molecule of any one of claims 27 to 30, wherein X₅ is Lys.

36. The proteinaceous molecule according to claims 27-31, wherein the proteinaceous molecule of Formula I comprises, consists, or consists essentially of, an amino acid sequence represented by any one of SEQ ID NO: 1 or 2:
SEQ ID NO: 1
GKKRTKTWHKGTLIAIQTVGPGKKYKVK
SEQ ID NO: 2
GKKRRKLWHGGTLIPIQTVGPGKKKKVK

37. The proteinaceous molecule according to any one of claims 27-36, wherein the proteinaceous molecule of Formula I further comprises at least one membrane permeating moiety.

38. The proteinaceous molecule according to claim 37, wherein the membrane permeating moiety is a lipid moiety.

39. The proteinaceous molecule according to claim 38, wherein the membrane permeating moiety is a myristoyl group.

40. The proteinaceous molecule according to any one of claims 33-39, wherein the membrane permeating moiety is coupled to the N- or C-terminal amino acid residue.

41. The proteinaceous molecule according to claim 40, wherein the membrane permeating moiety is coupled to the N-terminal amino acid residue.

42. A SETDB1 bicyclic peptide mimetic comprising a polypeptide that comprises at least three cysteine residues, separated by at least two loop sequences, and a molecular scaffold which forms covalent bonds with the cysteine residues of the polypeptide such that at least two polypeptide loops are formed on the molecular scaffold, wherein the SETDB1 bicyclic peptide mimetic comprises an amino acid sequence of Formula (II):
Formula (II)
Z₁X₁C₁GKKRTKTWHC₂KGTLIAIQTVGX₂GC₃KKYKVKZ₂
or a modified derivative, or pharmaceutically acceptable salt, thereof.
wherein:
C₁, C₂, and C₃ represent first, second and third cysteine residues, respectively;
Z₁ and Z₂ are independently absent or are independently selected from at least one of a proteinaceous moiety comprising from about 1 to about 50 amino acid residues (and all integer resides in between), and a protecting moiety;
X₁ is absent or alanine; and
X₂ is selected from any nonpolar/neutral amino acid residue (e.g., proline, leucine, alanine, glycine, isoleucine, methionine, phenylalanine, tryptophan, valine, and norleucine).

43. The SETDB1 bicyclic peptide mimetic of claim 42, wherein X₁ is absent.

44. The SETDB1 bicyclic peptide mimetic of claim 42 or claim 43, wherein X₂ is selected from proline or leucine.

45. The SETDB1 bicyclic peptide mimetic of any one of claims 42 to 44, wherein X₂ is proline.

46. The SETDB1 bicyclic peptide mimetic of any one of claims 42 to 45, wherein Z₁ is absent.

47. The SETDB1 bicyclic peptide mimetic of any one of claims 42 to 46, wherein Z₂ is absent.

48. The SETDB1 bicyclic peptide mimetic of any one of claims 42 to 47, wherein the peptide binds to importin.

49. The SETDB1 bicyclic peptide mimetic of any one of claims 42 to 48, wherein the peptide mimetic prevents or disrupts the complex between SETDB1 and importin.

50. The SETDB1 bicyclic peptide mimetic of any one of claims 42 to 49, comprising the amino acid sequence:
[SEQ ID NO: 8]
CGKKRTKTWHCKGTLIAIQTVGPGCKKYKV.

51. The SETDB1 bicyclic peptide mimetic of any one of claims 42 to 50, wherein the molecular scaffold is 1,3,5-(tribroMoMethyl)benzene) or TBAB.

52. The SETDB1 bicyclic peptide mimetic of any one of claims 42 to 51, further comprising an N-terminal cell-penetrating peptide.

53. The SETDB1 bicyclic peptide mimetic of claim 52, wherein the cell-penetrating peptide is Myr.

54. The SETDB1 bicyclic peptide mimetic of any one of claims 42 to 53, wherein the modified derivative includes one or more modifications selected from: N-terminal and/or C-terminal modifications; replacement of one or more amino acid residues with one or more non-natural amino acid residues (such a replacement of one or more polar amino acids with one or more isosteric or isoelectronic amino acids; replacement or one or more hydrophobic amino acid residues with other non-natural isosteric or isoelectronic amino acids); addition of a spacer group; replacement of one or more oxidation resistant amino acid residues; replacement of one or more amino acid residues with an alanine, replacement of one or more L amino acid residues with one or more D-amino acid residues; N-alkylation of one or more aminide bonds within the bicyclic peptide ligand; replacement of one or more peptide bonds with a surrogate bond; peptide backbone length modification; substitution of the hydrogen on the α-carbon of one or more amino acid residues with another chemical group, and post-synthetic biorthogonal modification of amino acids such as cysteine, lysine, glutamate and tyrosine with suitable amine, thiol, carboxylic acid and phenol-reactive reagents.

55. The SETDB1 bicyclic peptide mimetic of any one of claims 42 to 54, wherein the modified derivative comprises an N-terminal modification, such as an N-terminal acetyl group.

56. The SETDB1 bicyclic peptide mimetic of any one of claims 42 to 55, wherein the modified derivative comprises a C-terminal modification, such as a C-terminal amide group.

57. The SETDB1 bicyclic peptide mimetic of any one of claims 42 to 56, wherein the modified derivative comprises replacement of one or more amino acid residues with one or more non-natural amino acid residues.

58. The SETDB1 bicyclic peptide mimetic of any one of claims 42 to 57, wherein the modified derivative comprises one or more D-amino acid.

59. The SETDB1 bicyclic peptide mimetic of any one of claims 42 to 58, wherein substantially of the amino acids except glycine are D-amino acids.'

60. The SETDB1 bicyclic peptide mimetic of any one of claims 42 to 59, wherein two or more of the amino acids are in retro-inverso form.

61. The SETDB1 bicyclic peptide mimetic of any one of claims 42 to 60, wherein all of the amino acids are in retro-inverso form.

62. The SETDB1 bicyclic peptide mimetic of claim 61, the amino acid sequence set forth in SEQ ID NO: 11 (vkykkcGpgvtqisiltGkchwtktrkkcG).

63. The SETDB1 bicyclic peptide mimetic of any one of claims 42 to 62, wherein the pharmaceutically acceptable salt is selected from a hydrochloride or acetate salt.

64. A pharmaceutical composition comprising the SETDB1 bicyclic peptide mimetic of any one of claims 42 to 63, in combination with one or more excipients.

65. A method of reducing nuclear localization of SETDB1 in a SETDB1 overexpressing cell, the method comprising contacting the cell with a SETDB1 bicyclic peptide mimetic with the amino acid sequence of:
Formula (II)
Z₁X₁C₁GKKRTKTWHC₂KGTLIAIQTVGX₂GC₃KKYKVKZ₂
or a modified derivative, or pharmaceutically acceptable salt, thereof,
wherein:
C₁, C₂, and C₃ represent first, second and third cysteine residues, respectively;
Z₁ and Z₂ are independently absent or are independently selected from at least one of a proteinaceous moiety comprising from about 1 to about 50 amino acid residues (and all integer resides in between), and a protecting moiety;
X₁ is absent or alanine; and
X₂ is selected from any nonpolar/neutral amino acid residue (e.g., proline, leucine, alanine, glycine, isoleucine, methionine, phenylalanine, tryptophan, valine, and norleucine).

66. The SETDB1 bicyclic peptide mimetic of claim 65 wherein X₁ is absent.

67. The SETDB1 bicyclic peptide mimetic of claim 65 or claim 66, wherein X₂ is selected from proline or leucine.

68. The SETDB1 bicyclic peptide mimetic of any one of claims 65 to 67, wherein X₂ is proline.

69. The SETDB1 bicyclic peptide mimetic of any one of claims 65 to 68, wherein Z₁ is absent.

70. The SETDB1 bicyclic peptide mimetic of any one of claims 65 to 69, wherein Z₂ is absent.

71. The method of any one of claims 65-70, comprising the amino acid sequence:
[SEQ ID NO: 8]
CGKKRTKTWHCKGTLIAIQTVGPGCKKYKV.

72. The method of any one of claims 65-71, wherein the molecular scaffold is 1,3,5-(tribroMoMethyl)benzene), or TBAB.

73. The method of any one of claims 65-72, wherein the modified derivative comprises one or more D-amino acid.

74. The method of any one of claims 65-73, wherein substantially of the amino acids are D-amino acids.

75. The method of any one of claims 65 to 74, wherein the peptide is formulated in a lipid nanoparticle.

76. The method of any one of claims 65-75, further comprising an N-terminal cell-penetrating peptide.

77. The method of claim 76, wherein the cell-penetrating peptide is myristic acid.

78. A method of treating or preventing cancer in a subject, wherein the cancer comprises at least one SETDB1 overexpressing cell, comprising administering to the subject a SETDB1 bicyclic peptide mimetic of any one of claims 43-62.

79. The method of any one of claims 65-78, wherein the SETDB1 overexpressing cell is a cancer cell, cancer stem cell, or a non-cancer stem cell tumour cell.

80. The method of claim 78 or claim 79, wherein the cancer is selected from breast, prostate, lung, bladder, pancreatic, colon, liver, or brain cancer, or melanoma, or retinoblastoma.

81. The method of any one of claims 65 to 80, further comprising administering at least one further cancer therapy.

82. The method of claim 81, wherein the further cancer therapy is a chemotherapeutic agent and/or an immunotherapy.

83. The method of claim 81, wherein the immunotherapy is an immune checkpoint inhibitor.

84. A composition comprising a SETDB1 bicyclic peptide mimetic with the amino acid sequence of:
Formula (III)
Z₁X₁C₁GKKRTKTWHC₂KGTLIAIQTVGX₂GC₃KKYKVKZ₂
or a modified derivative, or pharmaceutically acceptable salt, thereof, for use in therapy;
wherein:
C₁, C₂, and C₃ represent first, second and third cysteine residues, respectively;
Z₁ and Z₂ are independently absent or are independently selected from at least one of a proteinaceous moiety comprising from about 1 to about 50 amino acid residues (and all integer resides in between), and a protecting moiety;
X₁ is absent or alanine; and
X₂ is selected from any nonpolar/neutral amino acid residue (e.g., proline, leucine, alanine, glycine, isoleucine, methionine, phenylalanine, tryptophan, valine, and norleucine).

85. The composition of claim 84 wherein X₁ is absent.

86. The composition of claim 84 or claim 85, wherein X₂ is selected from proline or leucine.

87. The composition of any one of claims 84 to 86, wherein X₂ is proline.

88. The composition of any one of claims 84 to 87, wherein Z₁ is absent.

89. The composition of any one of claims 84 to 88, wherein Z₂ is absent.

90. The composition of any one of claims 84 to 89, comprising the amino acid sequence:
[SEQ ID NO: 8]
CGKKRTKTWHCKGTLIAIQTVGPGCKKYKV.

91. A composition comprising a SETDB1 bicyclic peptide mimetic with the amino acid sequence of:
Formula (III)
Z₁X₁C₁GKKRTKTWHC₂KGTLIAIQTVGX₂GC₃KKYKVKZ₂
or a modified derivative, or pharmaceutically acceptable salt, thereof; for use in the treatment of cancer;
wherein:
C₁, C₂, and C₃ represent first, second and third cysteine residues, respectively;
Z₁ and Z₂ are independently absent or are independently selected from at least one of a proteinaceous moiety comprising from about 1 to about 50 amino acid residues (and all integer resides in between), and a protecting moiety;
X₁ is absent or alanine; and
X₂ is selected from any nonpolar/neutral amino acid residue (e.g., proline, leucine, alanine, glycine, isoleucine, methionine, phenylalanine, tryptophan, valine, and norleucine).

92. The composition of claim 91, wherein X₁ is absent.

93. The composition of claim 91 or claim 92, wherein X₂ is selected from proline or leucine.

94. The composition of any one of claims 91 to 93, wherein X₂ is proline.

95. The composition of any one of claims 91 to 94, wherein Z₁ is absent.

96. The composition of any one of claims 91 to 95, wherein Z₂ is absent.

97. The composition of any one of claims 91 to 96, comprising the amino acid sequence:
[SEQ ID NO: 8]
CGKKRTKTWHCKGTLIAIQTVGPGCKKYKV.

98. Use of a composition comprising a bicyclic peptide with the amino acid sequence:
Formula (III)
Z₁X₁C₁GKKRTKTWHC₂KGTLIAIQTVGX₂GC₃KKYKVKZ₂
or a modified derivative, or pharmaceutically acceptable salt, thereof; for use in the manufacture of a medicament for the treatment of cancer;
wherein:
C₁, C₂, and C₃ represent first, second and third cysteine residues, respectively;
Z₁ and Z₂ are independently absent or are independently selected from at least one of a proteinaceous moiety comprising from about 1 to about 50 amino acid residues (and all integer resides in between), and a protecting moiety;
X₁ is absent or alanine; and
X₂ is selected from any nonpolar/neutral amino acid residue (e.g., proline, leucine, alanine, glycine, isoleucine, methionine, phenylalanine, tryptophan, valine, and norleucine).

99. The use of claim 98, wherein X₁ is absent.

100. The use of claim 98 or claim 99, wherein X₂ is selected from proline or leucine.

101. The use of any one of claims 98 to 100, wherein X₂ is proline.

102. The use of any one of claims 98 to 101, wherein Z₁ is absent.

103. The use of any one of claims 98 to 102, wherein Z₂ is absent.

104. The composition of any one of claims 98 to 103, comprising the amino acid sequence:
[SEQ ID NO: 8]
CGKKRTKTWHCKGTLIAIQTVGPGCKKYKV.

105. A method of predicting the likelihood of response to a therapy (e.g., an immunotherapy) in a subject, the method comprising, consisting, or consisting essentially of analysing cellular localization of SETDB1 in a SETDB1 expressing cell of the subject, to thereby predict the likelihood of response of the patient to the therapy.

106. The method of claim 105, wherein the SETDB1-expressing cell is a tumour cell.

107. The method of claim 1 or claim 2, wherein therapy is an immunotherapy.

108. The method of any one of claims 105 to 107, comprising detecting the presence of SETDB1 in the nucleus of the cell, or a level of SETDB1 in the nucleus of the cell, which is indicative of an aberrant or abnormal nuclear level of SETDB1 and which correlates with an increased likelihood of resistance to the therapy, the thereby determine that the subject has increased likelihood of resistance to the therapy.

109. The method of claim 108, comprising detecting comparing the level of SETDB1 between different compartments (e.g., nucleus, cytoplasm) of the cell, to thereby determine that the subject has increased likelihood of resistance to therapy.

110. The method of claim 109, comprising detecting a higher level of SETDB1 relative to a control in the nucleus of the cell, the thereby determine that the subject has increased likelihood of resistance to the therapy.

111. The method of any one of claims 105 to 110, comprising comparing the level of SETDB1 between different cellular compartments (e.g., nucleus, cytoplasm) of the cell to thereby determine that the subject has increased likelihood if resistance to the therapy.

112. The method of any one of claims 105 to 111, comprising detecting a higher level of SETDB1 in the nucleus of the cell relative to a control (e.g., relative to the nucleus of a corresponding normal cell, or relative to the level of SETDB1 outside the nucleus of the subject's cell such as the cytoplasm of the cell), which indicates that the subject has increased likelihood of resistance to the therapy.

113. The method of claim 112, wherein the higher level of SETDB1 in the nucleus of the cell represents a level that is at least about 120%, 130%, 140%, 150%, 160%, 170%, 180%, 190%, 200%, 250%, 300%, 400%, 500%, 600%, 700%, 800%, 900%, or 1000% (and every integer in between) of the level os SETDB1 in the nucleus of the corresponding responsive control cell.

114. The method of claim 112 or claim 113, wherein the higher level of SETDB1 in the nucleus of the cell represents a higher level of SETDB1 in the nucleus of the cell than outside the nucleus (e.g., cytoplasm, or extranuclear) of the cell.

115. The method of claim 114, wherein the higher level is indicative of a ratio of nuclear SETDB1 to extranuclear SETDB1 of greater than about 0.55, 0.65, 0.70, 0.75, 0.85, 0.90, or 0.95.

116. The method of any one of claims 105 to 115, comprising detecting a higher level of nuclear SETDB1 in more than 30%, 35%, 40%, 45%, 50%, 55%, 60%, 65%, 70%, 75%, 80%, 85%, 90% or 95% of the subject's cells (e.g., tumour cells) which indicates that the subject has increased likelihood of resistance to the therapy.

117. The method of any one of claims 105 to 108, comprising detecting absence of SETDB1 in the nucleus of the cell or a level of SETDB1 in the nucleus of the cell which is indicative of a normal nuclear level of SETDB1 and which correlates with an increased likelihood of sensitivity to the therapy, to thereby determine that the subject has increased likelihood of sensitivity to the therapy.

118. The method of any one of claims 105 to 108 and 117, comprising detecting a level of SETDB1 in the nucleus of the cell relative to a control (e.g., relative to the nucleus of a corresponding normal cell, or relative to the levels of SETDB1 outside the nucleus of the subject's cell), which level is indicative of a normal nuclear level of SETDB1 and which indicates that the patient has increased likelihood of sensitivity to the therapy.

119. The method of any one of claims 105 to 108, 117, and 118, comprising detecting presence of SETDB1 outside the nucleus (e.g., cytoplasm) of the cell to thereby determine that the subject has increased likelihood of sensitivity to the therapy.

120. The method of any one of claims 105-108, 117, 118, and 119, comprising detecting a level of SETDB1 outside the nucleus of the cell relative to a control (e.g., relative to outside the nucleus of a corresponding normal cell, or relative to the levels of SETDB1 inside the nucleus of the subject's cell), which level is indicative of a normal extranuclear level of SETDB1, and which indicates that the patient has increased likelihood of sensitivity to the therapy.

121. The method of claim 119 or claim 120, wherein the level of SETDB1 outside the nucleus of the cell represents a level that is about the same level (e.g., a level that is from about 85% to about 115%, and every integer in between) of SETDB1 outside the nucleus of the corresponding normal control cell.

122. The method of any one of claim 119 to 121, wherein the level of SETDB1 outside the nucleus of the cell represents a higher level of SETDB1 outside the nucleus (e.g., surface and/or cytoplasm) of the cell than inside the nucleus of the cell.

123. The method of claim 122, wherein the higher level is indicative of a ratio of extranuclear SETDB1 to nuclear SETDB1 of greater than about 0.55, 0.65, 0.70, 0.75, 0.85, 0.90, or 0.95.

124. The method of any one of claims 105 to 108, and 117 to 123, comprising detecting a higher level of extranuclear SETDB1 in more than 30%, 35%, 40%, 45%, 50%, 55%, 60%, 65%, 70%, 75%, 80%, 85%, 90% or 95% of the subject's cells (e.g., tumour cells) which indicates that the subject has increased likelihood of sensitivity to the therapy.

125. The method of any one of claims 105 to 124, comprising detecting the presence, absence, or level of co-localization of SETDB2 with a nuclear binding partner of SETDB1 (e.g., ATF7IP).

126. The method of claim 125, comprising contacting a sample comprising a cell of the subject or lysate of the cell with a first antigen-binding molecule that binds specifically to SETDB1 and a second antigen-binding molecule that binds specifically to the nuclear binding partner, and detecting the presence in the sample of a complex that comprises SETDB1, the nuclear binding partner, the first antigen-binding molecule and the second antigen-binding molecule, to thereby determine that the subject has increased likelihood of resistance to the therapy.

127. The method of claim 126, comprising detecting a higher level of the complex relative to a control (e.g., a corresponding normal control cell), which indicates that the subject has increased likelihood of resistance to the therapy.

128. The method of claim 125, comprising detecting a level of the complex in the nucleus relative to a control (e.g., a corresponding normal control cell), which level is indicative of a normal level of the complex and which indicates that the subject has increased likelihood of sensitivity to the therapy.

129. A method for stratifying a subject as a likely responder or non-responder to a therapy (e.g., immunotherapy), the method comprising, consisting or consisting essentially of analyzing cellular localization of SETDB1 in a sample of the subject, as defined in any one of claims 105 to 128, to determine whether the subject has increased likelihood of sensitivity or resistance to the therapy, to thereby stratify the subject as a likely responder or non-responder to the therapy.

130. A method for managing treatment of a subject with a therapy (e.g., immunotherapy), the method comprising, consisting or consisting essentially of selecting a subject for treating with the therapy on the basis that the subject is a likely responder to the therapy, or selecting a subject for not treating with the therapy on the basis that the subject is a likely non-responder to the therapy and treating or not treating the subject with the therapy based on the selection, wherein the selection is based on the stratification method according to claim 129.

131. A method for predicting treatment outcome of a subject with a therapy (e.g., immunotherapy), the method comprising, consisting or consisting essentially of analyzing cellular localization of SETDB1 in a sample of the subject, as defined in any one of claims 105 to 128, to determine whether the subject has increased likelihood of sensitivity or resistance to the therapy, to thereby predict the treatment outcome for the patient.

132. A kit for detecting location of SETDB1 in a cellular location (e.g., cytoplasm or nucleus) of a cell, for predicting the likelihood of response of a cell to a therapy (e.g., immunotherapy), for determining likelihood of resistance of a subject to a therapy (e.g., immunotherapy), for determining likelihood of sensitivity of a subject to a therapy (e.g., immunotherapy), for stratifying a subject as a likely responder or non-responder to a therapy (e.g., immunotherapy), for managing treatment of a subject with a therapy (e.g., immunotherapy), for monitoring a disease in a subject following treatment with a therapy, for determining the status of a disease in a subject and/or for determining the immune status of a subject, the kit comprising, consisting or consisting essentially of a first antigen-binding molecule that binds specifically to SETDB1.

133. The kit of claim 132, further comprising a second antigen-binding molecule that binds specifically to a nuclear binding partner of SETDB1 (e.g., ATF7IP).

134. kit of claim 133, further comprising a third antigen-binding molecule, which suitably comprises a detectable label, that binds to the first and second antigen-binding molecules.

135. The kit of any one of claims 132 to 134, further comprising instructional material for performing the method of any one of claims 105 to 131.

136. The composition, method, or kit of any one of the preceding claims wherein all of the amino acids are D isoform (i.e., vkykkcGpgvtqisiltGkchwtktrkkcG) and in retro-inverso form.
